(19) United States
(12) Patent Application Publication
(10) Pub. No.: US 2023/0346468 A1
(43) Pub. Date: Nov. 2, 2023

(54) SYSTEMS AND METHODS THAT FACILITATE TISSUE TREATMENT BASED ON PROXIMITY INFORMATION

(71) Applicant: KARDIUM INC., Burnaby (CA)

(72) Inventors: Daniel Martin Reinders, Richmond (CA); Justin Aaron Michael, Vancouver (CA)

(21) Appl. No.: 18/308,000

(22) Filed: Apr. 27, 2023

Related U.S. Application Data
(60) Provisional application No. 63/336,070, filed on Apr. 28, 2022.
(60) Provisional application No. 63/399,803, filed on Aug. 22, 2022.

(51) Int. Cl. A61B 18/14

(52) U.S. Cl. CPC A61B 18/1492; A61B 2018/00577

(57) ABSTRACT

At least a first transducer set of a transducer-based device may be activated to deliver a first high voltage pulse set to cause pulsed field ablation of tissue. A data set may be monitored, the data set indicative of separation between a second transducer set of the transducer-based device and a tissue surface in a bodily cavity. Depending on a degree of separation, indicated by the data set, between the second transducer set and the tissue surface, a quality of a lesion producible in the tissue by the first high voltage pulse set may be determined. At least in response to the determination of the quality of the lesion producible in the tissue by the first high voltage pulse set, display of a graphical element set may be caused to indicate the determined quality of the lesion.

### CROSS-REFERENCE TO RELATED APPLICATIONS

This application claims the benefit of each of U.S. Provisional Application No. 63/336,070, filed Apr. 28, 2022 and U.S. Provisional Application No. 63/399,803, filed Aug. 22, 2022, the entire disclosure of each of these applications is hereby incorporated herein by reference.

### TECHNICAL FIELD

Aspects of this disclosure generally are related to systems and methods that facilitate tissue treatment based on transducer-to-tissue proximity information, according to some embodiments of the present invention.

### BACKGROUND

Cardiac surgery was initially undertaken using highly invasive open procedures. A sternotomy, which is a type of incision in the center of the chest that separates the sternum, was typically employed to allow access to the heart. In the past several decades, more and more cardiac operations are performed using intravascular or percutaneous techniques, where access to inner organs or other tissue is gained via a catheter.

Intravascular or percutaneous surgeries benefit patients by reducing surgery risk, complications and recovery time. However, the use of intravascular or percutaneous technologies also raises some particular challenges. Medical devices used in intravascular or percutaneous surgery need to be deployed via catheter systems which significantly increase the complexity of the device structure. As well, doctors do not have direct visual contact with the medical devices once the devices are positioned within the body.

One example of where intravascular or percutaneous medical techniques have been employed is in the treatment of a heart disorder called atrial fibrillation. Atrial fibrillation is a disorder in which spurious electrical signals cause an irregular heartbeat. Atrial fibrillation has been treated with open heart methods using a technique known as the “Cox-Maze procedure”. During this procedure, physicians create specific patterns of lesions in the left or right atria to block various paths taken by the spurious electrical signals. Such lesions were originally created using incisions, but are now typically created by ablating the tissue with various techniques including radio-frequency (“RF”) energy, microwave energy, laser energy, and cryogenic techniques. Recently, pulsed field ablation (“PFA”) techniques have been investigated in various tissue ablation procedures. In PFA, high voltage pulses with sub-second pulse durations are applied to target tissue. In some cases, the high voltage pulses form pores in cell membranes in a procedure sometimes referred to as electroporation. When the electroporation process is such that the formed pores are permanent in nature and consequently result in cell death, the process is referred to as irreversible electroporation by some. When the electroporation process is such that the formed pores are temporary in nature, and the cell survives the electroporation process, the process is referred to as reversible electroporation by some. Pulsed field ablation, because it refers to ablation of tissue, typically involves irreversible electroporation of target tissue. In some cases, PFA shows a specificity for certain tissues. That is, in some cases, PFA may ablate a certain tissue type, but not another tissue type.

The intravascular or percutaneous atrial fibrillation treatment procedure is performed with the lack of direct vision that is provided in open procedures. However, it is relatively complex to perform, because of the difficulty in correctly positioning various catheter devices intravascularly or percutaneously to create the lesions in the correct locations. The efficacy of the formed lesions typically depends on the proximity between various transducers employed by the catheter and the tissue that is to be ablated, as increasing degrees of separation may limit the lesion depth that may be potentially achieved in either RF ablation procedures or PFA procedures. It also is particularly important to know the position of the various transducers that will be creating the lesions relative to tissue. The continuity, transmurality, and placement of the lesion patterns that are formed can impact the ability to block paths taken within the heart by spurious electrical signals and, consequently, can impact whether or not the procedure is successful. Accordingly, the present inventors have recognized that a need in the art exists for improved methods and systems that, or are configured to, determine and possibly indicate a particular level of PFA energy that is to be delivered based at least on the particular degree of proximity of at least a part of a transducer-based device to a tissue surface.

Some conventional systems have attempted to address the problem of lack of visibility of an internal medical device associated with percutaneous or intravascular procedures. Some conventional systems rely on fluoroscopic imaging to view the location of an internal medical device or probe, but it has been recognized that such fluoroscopic imaging does not readily produce images of tissue within the bodily cavity in sufficient detail to assess the proximity to tissue (e.g., including separation from tissue, or contact with tissue), of a particular transducer or to identify particular anatomical landmarks within the bodily cavity. Some conventional systems generate a graphical model of a tissue surface defining a bodily cavity into which a medical device or probe is deployed based on data acquired from electric-potential-based navigation systems, electromagnetic-based navigation systems, or ultrasound-based navigation systems. Some of these conventional navigation systems rely on a three-dimensional (“3D”) location of the medical device or probe located in the particular bodily cavity that is to be modeled. Some of these conventional navigation systems may incorporate a user interface employed to show a 3D graphical representation, envelope, or model of the bodily cavity, which, in some of these conventional systems, is generated via a medical practitioner moving a part of the medical device or probe (which moves a corresponding transducer) from point to point along the tissue wall. Some of these conventional systems may compile this sequence of points and, from such points, build the 3D graphical model of the bodily cavity. This model may be combined with real-time sensing of a location of the medical device or probe to provide the user with an awareness of the location of the medical device or probe in the bodily cavity with improved accuracy over, e.g., mere use of fluoroscopy.

However, while such conventional systems may be able to provide an indication of a location of a percutaneously deployed device within a bodily cavity, the present inventors have recognized that conventional tissue ablation systems can be improved with better determinations of, and providing feedback to a user for, a quality of a tissue lesion formable by ablation performed at a particular location in the bodily cavity. Accordingly, the present inventors have recognized that a need in the art exists for improved methods and systems that, or are configured to determine and indicate the quality of a lesion that may be formed in tissue.

### SUMMARY

At least the above-discussed need is addressed, and technical solutions are achieved by various embodiments of the present invention. According to some embodiments, a transducer operation system may be summarized as including an input-output device, a memory device system storing a program, and a data processing device system communicatively connected to the input-output device system and the memory device system. According to some embodiments, the data processing device system may be configured at least by the program at least to cause, via the input-output device system, activation of at least a first transducer set of a transducer-based device to deliver a first high voltage pulse set to cause pulsed field ablation of tissue. According to some embodiments, the data processing device system may be configured at least by the program at least to cause, via the input-output device system, monitoring of a data set indicative of separation between a second transducer set of the transducer-based device and a tissue surface in a bodily cavity. According to some embodiments, the data processing device system may be configured at least by the program at least to cause, based at least on an analysis of the data set, (a) determination, at least in response to a first state in which the analysis of the data set is indicative of a first degree of separation between the second transducer set and the tissue surface, of a first quality of a lesion producible in the tissue by the first high voltage pulse set, and (b) determination, at least in response to a second state in which the analysis of the data set is indicative of a second degree of separation between the second transducer set and the tissue surface, of a second quality of the lesion producible in the tissue by the first high voltage pulse set. According to various embodiments, the second degree of separation may be different than the first degree of separation. According to various embodiments, the data processing device system may be configured at least by the program at least to cause, via the input-output device system, (i) at least in response to the determination of the first quality of the lesion producible in the tissue by the first high voltage pulse set, display of a first graphical element set indicating the determined first quality of the lesion, and (ii) at least in response to the determination of the second quality of the lesion producible in the tissue by the first high voltage pulse set, display of a second graphical element set indicating the determined second quality of the lesion.

In some embodiments, the input-output device system may include a device location tracking system, and the data processing device system may be configured at least by the program at least to cause, via the input-output device system, reception of a location signal set from the device location tracking system, the location signal set indicating a location of at least one transducer in the second transducer set. In some embodiments, the data set may be derived at least in part from the location signal set. In some embodiments, the device location tracking system may be configured to generate the location signal set at least in response to one or more electric fields producible by one or more devices of the device location tracking system. In some embodiments, the one or more devices of the device location tracking system may be configured to operate outside a body comprising the bodily cavity. In some embodiments, the device location tracking system may be configured to generate the location signal set at least in response to one or more magnetic fields producible by one or more devices of the device location tracking system. In some embodiments, the one or more devices of the device location tracking system may be configured to operate outside a body comprising the bodily cavity.

In some embodiments, the data processing device system may be configured at least by the program at least to cause display, via the input-output device system, of an envelope representing the bodily cavity and a representation of the transducer-based device located in proximity to the envelope. In some embodiments, the data processing device system may be configured at least by the program at least to derive the data set at least in part from an analysis of information corresponding to a distance between at least part of the representation of the transducer-based device and a portion of the envelope adjacent the at least part of the representation of the transducer-based device. In some embodiments, the input-output device system may include a device location tracking system. In some embodiments, the data processing device system may be configured at least by the program at least to perform the analysis of the information corresponding to the distance between the at least part of the representation of the transducer-based device and the portion of the envelope adjacent the at least part of the representation of the transducer-based device based at least on a location signal set provided by the device location tracking system. In some embodiments, the data processing device system may be configured at least by the program at least to determine a location of the at least part of the representation of the transducer-based device based at least on a first location signal set provided by the device location tracking system, and to determine a location of the portion of the envelope adjacent the at least part of the representation of the transducer-based device based at least on a second location signal set provided by the device location tracking system.

In some embodiments, the input-output device system comprises a third transducer set. In some embodiments, the third transducer set may include at least a proximity sensor configured to determine a distance from the proximity sensor to the tissue surface. In some embodiments, the data set indicative of separation between the second transducer set of the transducer-based device and the tissue surface in a bodily cavity may be determined based at least on an analysis of a signal set provided by the proximity sensor. In some embodiments, the proximity sensor may be an ultrasonic sensor. In some embodiments, the transducer-based device may include the proximity sensor.

In some embodiments, the second transducer set may be at least a part of the first transducer set. In some embodiments, each graphical element in (1) the first graphical element set, (2) the second graphical element set, or each of (1) and (2) may correspond to a respective transducer in the first transducer set. In some embodiments, each graphical element in (1) the first graphical element set, (2) the second graphical element set, or each of (1) and (2) may correspond to a location of a respective transducer in the first transducer set during delivery of the first high voltage pulse set. In some embodiments, the data processing device system may be configured at least by the program at least to cause display, via the input-output device system, of a map of the tissue surface, and cause display, via the input-output device system, of (1) the first graphical element set, (2) the second graphical element set, or each of (1) and (2) at one or more locations on the map of the tissue surface corresponding to one or more locations on the tissue surface at which at least part of the lesion is formed. In some embodiments, the data processing device system may be configured at least by the program at least to cause display, via the input-output device system of a map of the tissue surface, and cause display, via the input-output device system, of (1) the first graphical element set, (2) the second graphical element set, or each (1) and (2) at one or more locations on the map of the tissue surface corresponding to one or more locations where the first high voltage pulse set is delivered.

In some embodiments, each of the first graphical element set and the second graphical element set includes at least one particular graphical element, and the data processing device system may be configured at least by the program at least to cause, via the input-output device system, (iii) at least in response to the determination of the first quality of the lesion producible in the tissue by the first high voltage pulse set, display of the at least one particular graphical element with a first visual characteristic set, and (iv) at least in response to the determination of the second quality of the lesion producible in the tissue by the first high voltage pulse set, display of the at least one particular graphical element with a second visual characteristic set, the second visual characteristic set different than the first visual characteristic set. In some embodiments, the second graphical element set may be the first graphical element set, but includes a change in at least one visual characteristic to indicate a change in lesion quality from the first quality of the lesion to the second quality of the lesion. In some embodiments, the second graphical element set may be distinct from the first graphical element set.

In some embodiments, the data processing device system may be configured at least by the program at least to cause (1) the determination of the first quality of the lesion producible in the tissue by the first high voltage pulse set, (2) the determination of the second quality of the lesion producible in the tissue by the first high voltage pulse set, or each of (1) and (2), at least in response to a particular configuration of the first high voltage pulse set. In some embodiments, the first high voltage pulse set may have a first particular configuration in the first state and may have a second particular configuration in the second state, the second particular configuration of the first high voltage pulse set different than the first particular configuration of the first high voltage pulse set. In some embodiments, the data processing device system may be configured at least by the program at least to cause the determination of the first quality of the lesion producible in the tissue by the first high voltage pulse set at least in response to the first configuration of the first high voltage pulse set. In some embodiments, the data processing device system may be configured at least by the program at least to cause the determination of the second quality of the lesion producible in the tissue by the first high voltage pulse set at least in response to the second configuration of the first high voltage pulse set.

In some embodiments, the first high voltage pulse set may have a first particular configuration in the first state and may have a second particular configuration in the second state, the second particular configuration of the first high voltage pulse set different than the first particular configuration of the first high voltage pulse set. In some embodiments, the data processing device system may be configured at least by the program at least to cause the determination of the first quality of the lesion producible in the tissue by the first high voltage pulse set at least in response to the first configuration of the first high voltage pulse set. In some embodiments, the data processing device system may be configured at least by the program at least to cause the determination of the second quality of the lesion producible in the tissue by the first high voltage pulse set at least in response to the second configuration of the first high voltage pulse set. In some embodiments, the first configuration of the first high voltage pulse set may be configured to deliver a first amount of power, and the second configuration of the first high voltage pulse set may be configured to deliver a second amount of power different than the first amount of power. In some embodiments, the first configuration of the first high voltage pulse set may be configured to deliver a first total number of high voltage pulses, and the second configuration of the first high voltage pulse set may be configured to deliver a second total number of high voltage pulses different than the first total number of high voltage pulses. In some embodiments, the first configuration of the first high voltage pulse set may be configured to deliver a first pulse voltage for each of at least one pulse in the first high voltage pulse set, and the second configuration of the first high voltage pulse set may be configured to deliver a second pulse voltage for each of at least one pulse in the first high voltage pulse set, the second pulse voltage different than the first pulse voltage. In some embodiments, the first configuration of the first high voltage pulse set may be configured to cause the first high voltage pulse set to have a first total pulse delivery duration, and the second configuration of the first high voltage pulse set may be configured to cause the first high voltage pulse set to have a second total pulse delivery duration different than the first total pulse delivery duration.

In some embodiments, the data processing device system may be configured at least by the program at least to cause, via the input-output device system, the monitoring of the data set at least prior to the activation. In some embodiments, wherein the data processing device system may be configured at least by the program at least to cause, via the input-output device system, the monitoring of the data set at least during the activation. In some embodiments, the data processing device system may be configured at least by the program at least to cause, via the input-output device system, the monitoring of the data set at least after the activation.

According to various embodiments, different systems may include different combinations and sub-combinations of those described above.

According to some embodiments, a transducer operation system may be summarized as including an input-output device system, a memory device system storing a program, and a data processing device system communicatively connected to the input-output device system and the memory device system. According to various embodiments, the data processing device system may be configured at least by the program at least to cause, via the input-output device system, an operation of at least a first transducer set of a transducer-based device to deliver a first high voltage pulse set to cause pulsed field ablation of tissue. According to various embodiments, the data processing device system may be configured at least by the program at least to cause, via the input-output device system, monitoring of a first data set indicative of proximity between a second transducer set of the transducer-based device and a tissue surface in a bodily cavity. According to various embodiments, the data processing device system may be configured at least by the program at least to cause, based at least on an analysis of the first data set, determination, at least in response to a first state in which the analysis of the first data set is indicative of a first degree of proximity between the second transducer set and the tissue surface, of a first quality of a lesion producible in the tissue by the first high voltage pulse set. According to various embodiments, the data processing device system may be configured at least by the program at least to cause, via the input-output device system and at least in response to the determination of the first quality of the lesion producible in the tissue by the first high voltage pulse set, display of a first graphical element set indicating the determined first quality of the lesion. According to various embodiments, the data processing device system may be configured at least by the program at least to cause, via the input-output device system, an operation of at least a third transducer set of the transducer-based device to deliver a second high voltage pulse set to cause pulsed field ablation of the tissue, the delivery of the second high voltage pulse set occurring after the delivery of the first high voltage pulse set. According to various embodiments, the data processing device system may be configured at least by the program at least to cause determination of a second quality of the lesion producible in the tissue, the second quality of the lesion producible in the tissue indicating a cumulative effect on the tissue as a result of at least delivery of the first high voltage pulse set and the second high voltage pulse set. According to various embodiments, the data processing device system may be configured at least by the program at least to cause, via the input-output device system and at least in response to the determination of the second quality of the lesion, display of a second graphical element set indicating the determined second quality of the lesion.

In some embodiments, the data processing device system may be configured at least by the program at least to cause, via the input-output device system, monitoring of a second data set indicative of proximity between a fourth transducer set of the transducer-based device and the tissue surface of the bodily cavity. In some embodiments, the data processing device system may be configured at least by the program at least to cause the determination of the second quality of the lesion producible in the tissue based at least on an analysis of the second data set, the determination of the second quality of the lesion producible in the tissue made at least in response to a second state in which the analysis of the second data set is indicative of a second degree of proximity between the fourth transducer set and the tissue surface. In some embodiments, the data processing device system may be configured at least by the program at least to cause the monitoring of the second data set to occur at least in part after the delivery of the first high voltage pulse set.

In some embodiments, the fourth transducer set of the transducer-based device may be the second transducer set of the transducer-based device. In some embodiments, the second degree of proximity between the fourth transducer set and the tissue surface may be the same as the first degree of proximity between the second transducer set and the tissue surface. In some embodiments, the second degree of proximity between the fourth transducer set and the tissue surface may be different than the first degree of proximity between the second transducer set and the tissue surface. In some embodiments, (a) the second degree of proximity between the fourth transducer set and the tissue surface may indicate contact between at least one transducer in the fourth transducer set and the tissue surface, (b) the first degree of proximity between the second transducer set and the tissue surface may indicate contact between at least one transducer in the second transducer set and the tissue surface, or each of (a) and (b). In some embodiments, (a) the second degree of proximity between the fourth transducer set and the tissue surface may indicate separation between at least one transducer in the fourth transducer set and the tissue surface, (b) the first degree of proximity between the second transducer set and the tissue surface may indicate separation between at least one transducer in the second transducer set and the tissue surface, or each of (a) and (b). In some embodiments, each of the second transducer set and the third transducer set may be the first transducer set. In some embodiments, the fourth transducer set may be the third transducer set.

In some embodiments, the second quality of the lesion may indicate an enhanced degree of quality as compared to the first quality of the lesion. In some embodiments, the second quality of the lesion may indicate an enhanced degree of quality as compared to the first quality of the lesion. In some embodiments, the second quality of the lesion may indicate a greater degree of lesion size as compared to the first quality of the lesion. In some embodiments, the second quality of the lesion may indicate a greater degree of lesion depth as compared to the first quality of the lesion. In some embodiments, the third transducer set of the transducer-based device may be the first transducer set of the transducer-based device. In some embodiments, the third transducer set of the transducer-based device may be other than the first transducer set of the transducer-based device. In some embodiments, the data processing device system may be configured at least by the program at least to cause, via the input-output device system, monitoring of a third data set indicative of proximity between a location of at least a first transducer in the first transducer set at least at an inception or conclusion of, or during the delivery of the first high voltage pulse set and a location of at least a second transducer in the third transducer set at least at an inception or conclusion of, or during the delivery of the second high voltage pulse set. In some embodiments, the data processing device system may be configured at least by the program at least to cause determination of the second quality of the lesion producible in the tissue at least based on an analysis of the third data set.

In some embodiments, the second graphical element set may be the first graphical element set, but includes a change in at least one visual characteristic to indicate a change in lesion quality from the first quality of the lesion due to the delivery of the second high voltage pulse set. In some embodiments, the data processing device system may be configured at least by the program at least to cause the display of the second graphical element set indicating the determined second quality of the lesion by replacing the first graphical element set indicating the determined first quality of the lesion with the second graphical element set indicating the determined second quality of the lesion. In some embodiments, the displayed second graphical element set may be distinct from the displayed first graphical element set.

In some embodiments, the data processing device system may be configured at least by the program at least to cause, via the input-output device system, the monitoring of the first data set at least prior to the delivery of the first high voltage pulse set. In some embodiments, the data processing device system may be configured at least by the program at least to cause, via the input-output device system, the monitoring of the first data set at least during the delivery of the first high voltage pulse set. In some embodiments, the data processing device system may be configured at least by the program at least to cause, via the input-output device system, the monitoring of the first data set at least after the delivery of the first high voltage pulse set.

In some embodiments, the data processing device system may be configured at least by the program at least to cause, via the input-output device system, the monitoring of the second data set at least prior to the delivery of the second high voltage pulse set. In some embodiments, the data processing device system may be configured at least by the program at least to cause, via the input-output device system, the monitoring of the second data set at least during the delivery of the second high voltage pulse set. In some embodiments, the data processing device system may be configured at least by the program at least to cause, via the input-output device system, the monitoring of the second data set at least after the delivery of the second high voltage pulse set.

In some embodiments, the cumulative effect on the tissue may be a measured cumulative effect. In some embodiments, the cumulative effect on the tissue may be a predicted cumulative effect.

In some embodiments, the first high voltage pulse set and the second high voltage pulse set may form part of an uninterrupted high voltage pulse train. In some embodiments, the second high voltage pulse set may be temporally separated from the first high voltage pulse set by a third high voltage pulse set in the uninterrupted high voltage pulse train deliverable by the first transducer set of the transducer-based device. In some embodiments, the second high voltage pulse set may be temporally separated from the first high voltage pulse set by a third high voltage pulse set. In some embodiments, successive pulses in the first high voltage pulse set are temporally spaced according to a first period of time, and successive pulses in the second high voltage pulse set are temporally spaced according to a second period of time. In some embodiments, the second high voltage pulse set may be temporally separated from the first high voltage pulse set by a time interval that is greater than each of the first period of time and the second period of time.

In some embodiments, the input-output device system may include a device location tracking system. In some embodiments, the data processing device system may be configured at least by the program at least to determine location information of at least part of the transducer-based device based at least on a first location signal set provided by the device location tracking system, the location information indicating a change in location of the at least part of the transducer-based device during the delivery of the second high voltage pulse set as compared to a location of the at least part of the transducer-based device during the delivery of the first high voltage pulse set. In some embodiments, the at least part of the transducer-based device may include the third transducer set of the transducer-based device. In some embodiments, the third transducer set of the transducer-based device may be the first transducer set of the transducer-based device.

In some embodiments, the data processing device system may be configured at least by the program at least to cause, via the input-output device system, monitoring of a third data set indicative of movement of at least part of the transducer-based device, the third data set indicating a change in location of at least part of the transducer-based device from a time of the delivery of the first high voltage pulse set to a time of the delivery of the second high voltage pulse set. In some embodiments, the data processing device system may be configured at least by the program to determine the second quality of the lesion based at least on an analysis of the third data set.

According to various embodiments, different systems may include different combinations and sub-combinations of those described above.

According to some embodiments, a transducer operation system may be summarized as including an input-output device system, a memory device system storing a program, and a data processing device system communicatively connected to the input-output device system and the memory device system. In some embodiments, the data processing device system may be configured at least by the program at least to cause, via the input-output device system, a first operation of at least a first transducer set of a transducer-based device to deliver a first ablation energy to cause ablation of tissue. In some embodiments, the data processing device system may be configured at least by the program at least to cause, via the input-output device system, monitoring of a first data set indicative of proximity between a second transducer set of the transducer-based device and a tissue surface in a bodily cavity. In some embodiments, the data processing device system may be configured at least by the program at least to cause, based at least on an analysis of the first data set, determination, at least in response to a first state in which the analysis of the first data set is indicative of a first degree of proximity between the second transducer set and the tissue surface, of a first quality of a lesion producible in the tissue by the first ablation energy. In some embodiments, the data processing device system may be configured at least by the program at least to cause, via the input-output device system, and at least in response to the determination of the first quality of the lesion producible in the tissue by the first ablation energy, display of a graphical element set with a first visual characteristic set indicating the determined first quality of the lesion. In some embodiments, the data processing device system may be configured at least by the program at least to cause, via the input-output device system, a second operation of at least the first transducer set of the transducer-based device to deliver second ablation energy to cause ablation of the tissue, the delivery of the second ablation energy occurring after the delivery of the first ablation energy. In some embodiments, the data processing device system may be configured at least by the program at least to cause determination of a second quality of the lesion producible in the tissue, the second quality of the lesion producible in the tissue indicating a cumulative effect on the tissue as a result of at least delivery of the first ablation energy and the second ablation energy. In some embodiments, the data processing device system may be configured at least by the program at least to cause, via the input-output device system, and at least in response to the determination of the second quality of the lesion, display of the graphical element set with a second visual characteristic set indicating the determined second quality of the lesion.

In some embodiments the delivery of the first ablation energy and the delivery of the second ablation energy may form part of an uninterrupted delivery of ablation energy deliverable by the first transducer set of the transducer-based device. In some embodiments, the uninterrupted delivery of ablation energy may be an uninterrupted delivery of pulsed field ablation energy. In some embodiments, the uninterrupted delivery of ablation energy may be an uninterrupted delivery of radiofrequency (“RF”) ablation energy.

According to various embodiments, different systems may include different combinations and sub-combinations of those described above.

According to some embodiments, a tissue ablation system may be summarized as including an input-output device system, a memory device system storing a program, and a data processing device system communicatively connected to the input-output device system and the memory device system. According to various embodiments, the data processing device system may be configured at least by the program at least to receive, via the input-output device system, a data set indicative of proximity between at least part of a transducer-based device and tissue in a bodily cavity. In some embodiments, the data processing device system may be configured at least by the program at least to cause, via the input-output device system and the transducer-based device, initiation and then termination of delivery of a first train of pulses configured in accordance with a first pulse train parameter set at least in response to a first state in which at least part of the data set indicates that the part of the transducer-based device is in contact with a tissue surface in the bodily cavity. In some embodiments, a first activation time period exists from the initiation of the delivery of the first train of pulses to the termination of the delivery of the first train of pulses, and the first train of pulses is caused to be delivered during the first activation time period in accordance with the first pulse train parameter set to cause pulsed field tissue ablation. According to some embodiments, the first pulse train parameter set is configured to cause the first train of pulses to deliver a first total energy over the first activation time period. In some embodiments, the data processing device system may be configured at least by the program at least to cause, via the input-output device system and the transducer-based device, initiation and then termination of delivery of a second train of pulses configured in accordance with a second pulse train parameter set at least in response to a second state in which the at least part of the data set indicates that the part of the transducer-based device is separated from the tissue surface in the bodily cavity. In some embodiments, the second pulse train parameter set is different than the first pulse train parameter set. In some embodiments, a second activation time period exists from the initiation of the delivery of the second train of pulses to the termination of the delivery of the second train of pulses, and the second train of pulses is caused to be delivered during the second activation time period in accordance with the second pulse train parameter set to cause pulsed field tissue ablation. According to various embodiments, the second pulse train parameter set is configured to cause the second train of pulses to deliver a second total energy over the second activation time period that is greater than the first total energy.

In some embodiments, the first pulse train parameter set may be configured to cause the first train of pulses to deliver a first total number of pulses throughout the first activation time period in response to the first state, and the second pulse train parameter set is configured to cause the second train of pulses to deliver a second total number of pulses throughout the second activation time period in response to the second state. According to various embodiments, the second total number of pulses may be greater than the first total number of pulses. In some embodiments, the first pulse train parameter set may be configured to cause each pulse in the first train of pulses to have a first pulse configuration in response to the first state, and the second pulse train parameter set may configured to cause each pulse in the second train of pulses to have a second pulse configuration in response to the second state. According to various embodiments, the second pulse configuration may be the same as the first pulse configuration. In some embodiments, the first pulse configuration and the second pulse configuration may define a same pulse voltage. In some embodiments, the first pulse configuration and the second pulse configuration may define a same pulse width.

In some embodiments, the first pulse train parameter set may be configured to cause each of at least some of the pulses in the first train of pulses to have a first voltage in response to the first state, and the second pulse train parameter set may be configured to cause each of at least some pulses in the second train of pulses to have a second voltage. According to various embodiments, the second voltage may be greater than the first voltage. In some embodiments, the first pulse train parameter set may be configured to cause each of at least some pulses in the first train of pulses to have a first pulse width in response to the first state, and the second pulse train parameter set may be configured to cause each of at least some pulses in the second train of pulses to have a second pulse width in response to the second state. According to various embodiments, the second pulse width may be greater than the first pulse width. In some embodiments, the first pulse train parameter set may be configured to cause pulses in at least a portion of the first train of pulses to be delivered with a first pulse frequency in response to the first state, and the second pulse train parameter set may be configured to cause pulses in at least a portion of the second train of pulses to be delivered with a second pulse frequency in response to the second state. According to various embodiments, the second pulse frequency may be greater than the first pulse frequency.

In some embodiments, a duration of the second activation time period may be greater than a duration of the first activation time period. In some embodiments, a duration of the second activation time period may be equal to a duration of the first activation time period.

In some embodiments, the input-output device system may be configured to receive the data set at least in part from a contact sensing system. In some embodiments, the contact sensing system may include a force sensing system configured to determine a degree of contact force between the part of the transducer-based device and the tissue surface in the bodily cavity. In some embodiments, the contact sensing system may include a flow sensing system configured to determine a degree of contact between the part of the transducer-based device and the tissue surface in the bodily cavity.

In some embodiments, the input-output device system may be configured to interface with a device location tracking system, and the data processing device system may be configured at least by the program at least to receive, via the input-output device system, a location signal set from the device location tracking system, the location signal set indicating a location of at least a portion of the transducer-based device. In some embodiments, the data set may be derived at least in part from the location signal set. In some embodiments, the device location tracking system may be configured to generate the location signal set at least in response to one or more electric fields producible by one or more devices of the device location tracking system. In some embodiments, the device location tracking system may be configured to generate the location signal set at least in response to one or more magnetic fields producible by one or more devices of the device location tracking system.

In some embodiments, the data processing device system may be configured at least by the program at least to cause display, via the input-output device system, of an envelope representing the bodily cavity and a representation of the transducer-based device located in proximity to the envelope. In some embodiments, the data processing device system may be configured at least by the program at least to derive the data set at least in part from an analysis of information corresponding to a distance between at least part of the representation of the transducer-based device and a portion of the envelope adjacent the at least part of the representation of the transducer-based device. In some embodiments, the input-output device system may be configured to interface with a device location tracking system, and the data processing device system may be configured at least by the program at least to perform the analysis of the information corresponding to the distance between the at least part of the representation of the transducer-based device and the portion of the envelope adjacent the at least part of the representation of the transducer-based device based at least on a location signal set provided by the device location tracking system. In some embodiments, the input-output device system may be configured to interface with a device location tracking system, and the data processing device system may be configured at least by the program at least to determine a location of the at least part of the representation of the transducer-based device based at least on a first location signal set provided by the device location tracking system, and to determine a location of the portion of the envelope adjacent the at least part of the representation of the transducer-based device based at least on a second location signal set provided by the device location tracking system.

In some embodiments, the input-output device system may be configured to interface with a proximity sensor configured to determine a distance from the proximity sensor to the tissue surface. In some embodiments, the data set may be determined based at least on an analysis of a signal set provided by the proximity sensor. In some embodiments, the proximity sensor may be an ultrasonic sensor. In some embodiments, the transducer-based device may include the proximity sensor.

In some embodiments, the data set may include first data indicating a particular one of several possible degrees of contact between the part of the transducer-based device and the tissue surface in the bodily cavity, each of the several possible degrees of contact indicating some amount of contact between the part of the transducer-based device and the tissue surface in the bodily cavity. In some embodiments, the first pulse train parameter set may be configured to cause the first train of pulses to vary the first total energy delivered over the first activation time period in accordance with different degrees of contact between the part of the transducer-based device and the tissue surface in the bodily cavity indicated by the first data. In some embodiments, the first total energy, regardless of a manner in which it is varied according to the first pulse train parameter set in accordance with the different degrees of contact, is less than the second total energy. In some embodiments, the first pulse train parameter set may be configured to cause the first train of pulses to vary in pulse voltage to vary the first total energy delivered over the first activation time period in accordance with the different degrees of contact between the part of the transducer-based device and the tissue surface in the bodily cavity indicated by the first data. In some embodiments, the first pulse train parameter set may be configured to cause the first train of pulses to vary in pulse width to vary the first total energy delivered over the first activation time period in accordance with the different degrees of contact between the part of the transducer-based device and the tissue surface in the bodily cavity indicated by the first data. In some embodiments, the first pulse train parameter set may be configured to cause the first train of pulses to vary in pulse frequency to vary the first total energy delivered over the first activation time period in accordance with the different degrees of contact between the part of the transducer-based device and the tissue surface in the bodily cavity indicated by the first data.

In some embodiments, the data set may include second data indicating a particular one of several possible degrees of separation between the at least the part of the transducer-based device and the tissue surface in the bodily cavity, each of the several possible degrees of separation indicating some amount of separation between the part of the transducer-based device and the tissue surface in the bodily cavity. The second pulse train parameter set may be configured to cause the second train of pulses to vary the second total energy delivered over the second activation time period in accordance with different degrees of separation between the part of the transducer-based device and the tissue surface in the bodily cavity indicated by the second data. In some embodiments, the second total energy, regardless of a manner in which it is varied according to the second pulse train parameter set in accordance with the different degrees of separation, may be greater than the first total energy. In some embodiments, the second pulse train parameter set may be configured to cause the second train of pulses to vary in pulse voltage to vary the second total energy delivered over the second activation time period in accordance with the different degrees of separation between the part of the transducer-based device and the tissue surface in the bodily cavity indicated by the second data. In some embodiments, the second pulse train parameter set may be configured to cause the second train of pulses to vary in pulse width to vary the second total energy delivered over the second activation time period in accordance with the different degrees of separation between the part of the transducer-based device and the tissue surface in the bodily cavity indicated by the second data. In some embodiments, the second pulse train parameter set may be configured to cause the second train of pulses to vary in pulse frequency to vary the second total energy delivered over the second activation time period in accordance with the different degrees of separation between the part of the transducer-based device and the tissue surface in the bodily cavity indicated by the second data.

In some embodiments, the part of the transducer-based device is a first part of the transducer-based device, and the data processing device system may be configured at least by the program at least to cause, via the input-output device system and the transducer-based device, initiation and then termination of delivery of a third train of pulses configured in accordance with a third pulse train parameter set at least in response to a third state. In some embodiments, the third state is one in which at least a portion of the data set indicates that a second part of the transducer-based device other than the first part of the transducer-based device is separated from the tissue surface in the bodily cavity. In some embodiments, the third state may occur concurrently with the first state. In some embodiments, a third activation time period exists from the initiation of the delivery of the third train of pulses to the termination of the delivery of the third train of pulses, and the third train of pulses may be caused to be delivered during the third activation time period in accordance with the third pulse train parameter set to cause pulsed field tissue ablation. In some embodiments, the third pulse train parameter set may be configured to cause the third train of pulses to deliver a third total energy over the third activation time period that is greater than the first total energy. In some embodiments, the first activation time period and the third activation time period overlap.

According to various embodiments, different systems may include different combinations and sub-combinations of those described above.

Various embodiments of the present invention may include systems, devices, or machines that are or include combinations or subsets of any one or more of the systems, devices, or machines and associated features thereof summarized above or otherwise described herein (which should be deemed to include the figures).

Further, all or part of any one or more of the systems, devices, or machines summarized above or otherwise described herein or combinations or sub-combinations thereof may implement or execute all or part of any one or more of the processes or methods described herein or combinations or sub-combinations thereof.

According to some embodiments, a method may be executed by a data processing device system according to a program stored by a communicatively connected memory device system, the data processing device system also communicatively connected to an input-output device system, and the method may include: activating, via the input-output device system, at least a first transducer set of a transducer-based device to deliver a first high voltage pulse set to cause pulsed field ablation of tissue; monitoring, via the input-output device system, a data set indicative of separation between a second transducer set of the transducer-based device and a tissue surface in a bodily cavity; determining, at least in response to a first state in which an analysis of the data set is indicative of a first degree of separation between the second transducer set and the tissue surface, a first quality of a lesion producible in the tissue by the first high voltage pulse set; determining, at least in response to a second state in which an analysis of the data set is indicative of a second degree of separation between the second transducer set and the tissue surface, a second quality of the lesion producible in the tissue by the first high voltage pulse set, the second degree of separation different than the first degree of separation, and the second quality of the lesion different than the first quality of the lesion; displaying, via the input-output device system and at least in response to the determination of the first quality of the lesion producible in the tissue by the first high voltage pulse set, a first graphical element set indicating the determined first quality of the lesion; and displaying, via the input-output device system and at least in response to the determination of the second quality of the lesion producible in the tissue by the first high voltage pulse set, a second graphical element set indicating the determined second quality of the lesion.

According to some embodiments, a method may be executed by a data processing device system according to a program stored by a communicatively connected memory device system, the data processing device system also communicatively connected to an input-output device system, and the method may include: operating, via the input-output device system, at least a first transducer set of a transducer-based device to deliver a first high voltage pulse set to cause pulsed field ablation of tissue; monitoring, via the input-output device system, a first data set indicative of proximity between a second transducer set of the transducer-based device and a tissue surface in a bodily cavity; determining, based at least on an analysis of the first data set and at least in response to a first state in which the analysis of the first data set is indicative of a first degree of proximity between the second transducer set and the tissue surface, a first quality of a lesion producible in the tissue by the first high voltage pulse set; displaying, via the input-output device system and at least in response to the determination of the first quality of the lesion producible in the tissue by the first high voltage pulse set, a first graphical element set indicating the determined first quality of the lesion; operating, via the input-output device system, at least a third transducer set of the transducer-based device to deliver a second high voltage pulse set to cause pulsed field ablation of the tissue, the delivery of the second high voltage pulse set occurring after the delivery of the first high voltage pulse set; determining a second quality of the lesion producible in the tissue, the second quality of the lesion producible in the tissue indicating a cumulative effect on the tissue as a result of at least delivery of the first high voltage pulse set and the second high voltage pulse set; and displaying, via the input-output device system and at least in response to the determination of the second quality of the lesion, a second graphical element set indicating the determined second quality of the lesion.

According to some embodiments, a method may be executed by a data processing device system according to a program stored by a communicatively connected memory device system, the data processing device system also communicatively connected to an input-output device system, and the method may include: operating, via the input-output device system, at least a first transducer set of a transducer-based device to deliver a first ablation energy to cause ablation of tissue; monitoring, via the input-output device system, a first data set indicative of proximity between a second transducer set of the transducer-based device and a tissue surface in a bodily cavity; determining, based at least on an analysis of the first data set and at least in response to a first state in which the analysis of the first data set is indicative of a first degree of proximity between the second transducer set and the tissue surface, a first quality of a lesion producible in the tissue by the first ablation energy; displaying, via the input-output device system and at least in response to the determination of the first quality of the lesion producible in the tissue by the first ablation energy, a graphical element set with a first visual characteristic set indicating the determined first quality of the lesion; operating, via the input-output device system, at least the first transducer set of the transducer-based device to deliver second ablation energy to cause ablation of the tissue, the delivery of the second ablation energy occurring after the delivery of the first ablation energy; determining a second quality of the lesion producible in the tissue, the second quality of the lesion producible in the tissue indicating a cumulative effect on the tissue as a result of at least delivery of the first ablation energy and the second ablation energy; and displaying, via the input-output device system and at least in response to the determination of the second quality of the lesion, the graphical element set with a second visual characteristic set indicating the determined second quality of the lesion.

According to some embodiments, a method may be executed by a data processing device system according to a program stored by a communicatively connected memory device system, the data processing device system also communicatively connected to an input-output device system, and the method may include: receiving, via the input-output device system, a data set indicative of proximity between at least part of a transducer-based device and tissue in a bodily cavity; causing, via the input-output device system and the transducer-based device, initiation and then termination of delivery of a first train of pulses configured in accordance with a first pulse train parameter set at least in response to a first state in which at least part of the data set indicates that the part of the transducer-based device is in contact with a tissue surface in the bodily cavity, a first activation time period existing from the initiation of the delivery of the first train of pulses to the termination of the delivery of the first train of pulses, wherein the first train of pulses is caused to be delivered during the first activation time period in accordance with the first pulse train parameter set to cause pulsed field tissue ablation, and the first pulse train parameter set is configured to cause the first train of pulses to deliver a first total energy over the first activation time period; and causing, via the input-output device system and the transducer-based device, initiation and then termination of delivery of a second train of pulses configured in accordance with a second pulse train parameter set at least in response to a second state in which the at least part of the data set indicates that the part of the transducer-based device is separated from the tissue surface in the bodily cavity, the second pulse train parameter set different than the first pulse train parameter set, and a second activation time period existing from the initiation of the delivery of the second train of pulses to the termination of the delivery of the second train of pulses, wherein the second train of pulses is caused to be delivered during the second activation time period in accordance with the second pulse train parameter set to cause pulsed field tissue ablation, and the second pulse train parameter set is configured to cause the second train of pulses to deliver a second total energy over the second activation time period that is greater than the first total energy.

It should be noted that various embodiments of the present invention include variations of the methods or processes summarized above or otherwise described herein (which should be deemed to include the figures) and, accordingly, are not limited to the actions described or shown in the figures or their ordering, and not all actions shown or described are required according to various embodiments. According to various embodiments, such methods may include more or fewer actions and different orderings of actions. Any of the features of all or part of any one or more of the methods or processes summarized above or otherwise described herein may be combined with any of the other features of all or part of any one or more of the methods or processes summarized above or otherwise described herein.

In addition, a computer program product may be provided that includes program code portions for performing some or all of any one or more of the methods or processes and associated features thereof described herein, when the computer program product is executed by a computer or other computing device or device system. Such a computer program product may be stored on one or more computer-readable storage mediums, also referred to as one or more computer-readable data storage mediums or a computer-readable storage medium system.

In some embodiments, one or more computer-readable storage mediums may store a program executable by a data processing device system communicatively connected to an input-output device system. The program may include activation instructions configured to cause, via the input-output device system, activation of at least a first transducer set of a transducer-based device to deliver a first high voltage pulse set to cause pulsed field ablation of tissue. The program may include monitoring instructions configured to cause, via the input-output device system, monitoring of a data set indicative of separation between a second transducer set of the transducer-based device and a tissue surface in a bodily cavity. The program may include determination instructions configured to cause, based at least on an analysis of the data set, (a) determination, at least in response to a first state in which the analysis of the data set is indicative of a first degree of separation between the second transducer set and the tissue surface, of a first quality of a lesion producible in the tissue by the first high voltage pulse set, and (b) determination, at least in response to a second state in which the analysis of the data set is indicative of a second degree of separation between the second transducer set and the tissue surface, of a second quality of the lesion producible in the tissue by the first high voltage pulse set, the second degree of separation different than the first degree of separation, and the second quality of the lesion different than the first quality of the lesion. The program may include display instructions configured to cause, via the input-output device system, (i) at least in response to the determination of the first quality of the lesion producible in the tissue by the first high voltage pulse set, display of a first graphical element set indicating the determined first quality of the lesion, and (ii) at least in response to the determination of the second quality of the lesion producible in the tissue by the first high voltage pulse set, display of a second graphical element set indicating the determined second quality of the lesion.

In some embodiments, one or more computer-readable storage mediums may store a program executable by a data processing device system communicatively connected to an input-output device system. The program may include first operation instructions configured to cause, via the input-output device system, an operation of at least a first transducer set of a transducer-based device to deliver a first high voltage pulse set to cause pulsed field ablation of tissue. The program may include monitoring instructions configured to cause, via the input-output device system, monitoring of a first data set indicative of proximity between a second transducer set of the transducer-based device and a tissue surface in a bodily cavity. The program may include first determination instructions configured to cause, based at least on an analysis of the first data set, determination, at least in response to a first state in which the analysis of the first data set is indicative of a first degree of proximity between the second transducer set and the tissue surface, of a first quality of a lesion producible in the tissue by the first high voltage pulse set. The program may include first display instructions configured to cause, via the input-output device system and at least in response to the determination of the first quality of the lesion producible in the tissue by the first high voltage pulse set, display of a first graphical element set indicating the determined first quality of the lesion. The program may include second operation instructions configured to cause, via the input-output device system, an operation of at least a third transducer set of the transducer-based device to deliver a second high voltage pulse set to cause pulsed field ablation of the tissue, the delivery of the second high voltage pulse set occurring after the delivery of the first high voltage pulse set. The program may include second determination instructions configured to cause determination of a second quality of the lesion producible in the tissue, the second quality of the lesion producible in the tissue indicating a cumulative effect on the tissue as a result of at least delivery of the first high voltage pulse set and the second high voltage pulse set. The program may include second display instructions configured to cause, via the input-output device system and at least in response to the determination of the second quality of the lesion, display of a second graphical element set indicating the determined second quality of the lesion.

In some embodiments, one or more computer-readable storage mediums may store a program executable by a data processing device system communicatively connected to an input-output device system. The program may include first operation instructions configured to cause, via the input-output device system, a first operation of at least a first transducer set of a transducer-based device to deliver a first ablation energy to cause ablation of tissue. The program may include monitoring instructions configured to cause, via the input-output device system, monitoring of a first data set indicative of proximity between a second transducer set of the transducer-based device and a tissue surface in a bodily cavity. The program may include first determination instructions configured to cause, based at least on an analysis of the first data set, determination, at least in response to a first state in which the analysis of the first data set is indicative of a first degree of proximity between the second transducer set and the tissue surface, of a first quality of a lesion producible in the tissue by the first ablation energy. The program may include first display instructions configured to cause, via the input-output device system and at least in response to the determination of the first quality of the lesion producible in the tissue by the first ablation energy, display of a graphical element set with a first visual characteristic set indicating the determined first quality of the lesion. The program may include second operation instructions configured to cause, via the input-output device system, a second operation of at least the first transducer set of the transducer-based device to deliver second ablation energy to cause ablation of the tissue, the delivery of the second ablation energy occurring after the delivery of the first ablation energy. The program may include second determination instructions configured to cause determination of a second quality of the lesion producible in the tissue, the second quality of the lesion producible in the tissue indicating a cumulative effect on the tissue as a result of at least delivery of the first ablation energy and the second ablation energy. The program may include second display instructions configured to cause, via the input-output device system, and at least in response to the determination of the second quality of the lesion, display of the graphical element set with a second visual characteristic set indicating the determined second quality of the lesion.

In some embodiments, one or more computer-readable storage mediums may store a program executable by a data processing device system communicatively connected to an input-output device system. The program may include reception instructions configured to cause, via the input-output device system, reception of a data set indicative of proximity between at least part of a transducer-based device and tissue in a bodily cavity; first delivery instructions configured to cause, via the input-output device system and the transducer-based device, initiation and then termination of delivery of a first train of pulses configured in accordance with a first pulse train parameter set at least in response to a first state in which at least part of the data set indicates that the part of the transducer-based device is in contact with a tissue surface in the bodily cavity, a first activation time period existing from the initiation of the delivery of the first train of pulses to the termination of the delivery of the first train of pulses, wherein the first train of pulses is caused to be delivered during the first activation time period in accordance with the first pulse train parameter set to cause pulsed field tissue ablation, and the first pulse train parameter set is configured to cause the first train of pulses to deliver a first total energy over the first activation time period; and second delivery instructions configured to cause, via the input-output device system and the transducer-based device, initiation and then termination of delivery of a second train of pulses configured in accordance with a second pulse train parameter set at least in response to a second state in which the at least part of the data set indicates that the part of the transducer-based device is separated from the tissue surface in the bodily cavity, the second pulse train parameter set different than the first pulse train parameter set, and a second activation time period existing from the initiation of the delivery of the second train of pulses to the termination of the delivery of the second train of pulses, wherein the second train of pulses is caused to be delivered during the second activation time period in accordance with the second pulse train parameter set to cause pulsed field tissue ablation, and the second pulse train parameter set is configured to cause the second train of pulses to deliver a second total energy over the second activation time period that is greater than the first total energy.

In some embodiments, each of any of one or more or all of the computer-readable data storage mediums or medium systems (also referred to as processor-accessible memory device systems) described herein is a non-transitory computer-readable (or processor-accessible) data storage medium or medium system (or memory device system) including or consisting of one or more non-transitory computer-readable (or processor-accessible) storage mediums (or memory devices) storing the respective program(s) which may configure a data processing device system to execute some or all of any of one or more of the methods or processes described herein.

Further, any of all or part of one or more of the methods or processes and associated features thereof discussed herein may be implemented or executed on or by all or part of a device system, apparatus, or machine, such as all or a part of any of one or more of the systems, apparatuses, or machines described herein or a combination or sub-combination thereof.

### BRIEF DESCRIPTION OF THE DRAWINGS

It is to be understood that the attached drawings are for purposes of illustrating aspects of various embodiments and may include elements that are not to scale.

FIG. 1 includes a schematic representation of a transducer operation system, according to various example embodiments, the transducer operation system configured to perform tissue ablation, the transducer operation system including a data processing device system, an input-output device system, and a memory device system.

FIG. 2 includes a partially schematic representation of some particular implementations of a catheter navigation system or device location tracking system implementing an electric-field-based location system, according to various example embodiments.

FIG. 3 includes a partially schematic representation of some particular implementations of a catheter navigation system or device location tracking system implementing a magnetic-field-based location system, according to various example embodiments.

FIG. 4 includes a cutaway diagram of a heart showing a catheter navigation system or device location tracking system including a reference device and a transducer-based device of a transducer operation system configured to perform tissue ablation, the reference device percutaneously placed at least proximate a heart cavity, and the transducer-based device percutaneously placed in a left atrium of the heart, according to various example embodiments.

FIG. 5 includes a partially schematic representation of at least a portion of a transducer operation system configured to perform tissue ablation, according to various example embodiments, the transducer operation system including a data processing device system, an input-output device system, a memory device system, and a transducer-based device, the transducer-based device including a plurality of transducers and an expandable structure shown in a delivery or unexpanded configuration, according to various example embodiments.

FIG. 6 includes the representation of the portion of the transducer operation system of FIG. 5 with the expandable structure shown in a deployed or expanded configuration, according to various example embodiments.

FIG. 7 includes a schematic representation of a transducer-based device of a transducer operation system configured to perform tissue ablation, the transducer operation system including a flexible circuit structure, according to various example embodiments.

FIGS. 8A, 8B, 8C, and 8D illustrate program configurations of a transducer operation system or methods of operating a transducer operation system, according to various example embodiments.

FIG. 9A illustrates a graphical representation including a transducer-based device representation and a computer-generated envelope illustrating a mapped portion of a bodily cavity, according to various example embodiments.

FIG. 9B illustrates the graphical representation of FIG. 9A in a first state in which a particular transducer set exhibits a first degree of separation from the tissue surface of the bodily cavity, according to various example embodiments.

FIG. 9C illustrates the graphical representation of FIG. 9A in a second state in which the particular transducer set exhibits a second degree of separation from the tissue surface of the bodily cavity, according to various example embodiments.

FIG. 9D illustrates the graphical representation of FIG. 9A in which a graphical element is illustrated as being located on a tissue wall of the bodily cavity and represents a first quality of a lesion formed at such location, according to various example embodiments.

FIG. 9E illustrates the graphical representation of FIG. 9A in which a graphical element is illustrated as being located on a tissue wall of the bodily cavity and represents a second quality of a lesion formed at such location, according to various example embodiments.

FIG. 9F illustrates the graphical representation of FIG. 9A in which a graphical element illustrates a cumulative effect on tissue lesion quality from the application of multiple high voltage pulses, according to various example embodiments.

FIGS. 9G-1 and 9G-2 illustrate a simplified version of a portion of the graphical representation of FIG. 9A in which a graphical element set illustrates a cumulative effect on tissue lesion quality from the application of multiple high voltage pulse sets in a state in which relative movement occurs between a location of a prior delivery of a high voltage pulse set and a location of a later delivery of a high voltage pulse set, according to various example embodiments.

FIG. 9H illustrates a graphical representation like that of FIG. 9A, but instead of showing a computer-generated envelope representing a mapped portion of a bodily cavity, the bodily cavity is represented by a computerized-tomography (“CT”) scan image, according to various example embodiments.

Each of FIG. 10A, FIG. 10B, and FIG. 10C illustrates a respective simplified example of a train of pulses, according to various example embodiments.

### DETAILED DESCRIPTION

At least some embodiments of the present invention improve upon safety, efficiency, and effectiveness of various tissue ablation systems and methods of operation thereof. In some embodiments, the tissue ablation systems include transducer operation systems that include one or more transducers configured to perform tissue ablation. In some embodiments, the one or more transducers are configured to perform pulsed field ablation (“PFA”). In some embodiments, the one or more transducers are configured to perform thermal ablation (e.g., RF ablation). In some embodiments, the improved systems and methods include improved determinations and indications of the quality of a lesion that may be formed in tissue. In some embodiments, the improved systems and methods include improved determinations and indications of tissue lesion quality at least in one or more various states in which an ablation transducer is separated from (not in contact with) tissue. The inventors have recognized that conventional systems are lacking in determining lesion quality especially when an ablation transducer may be separated from the tissue that is to be ablated. In some embodiments, the improved systems and methods include improved determinations and indications of tissue lesion quality at least in one or more various states in which cumulative effects of multiple ablations or an extended-duration ablation is performed on a tissue region. The inventors have recognized that conventional systems are lacking in determining lesion quality especially when ablation is performed multiple times or for a particularly extended period of time on a particular tissue region. By more effectively determining and indicating to a user tissue lesion quality, the user can be provided with a more accurate and realistic understanding of an actual tissue lesion formed or expected to be formed, and occurrences of under- and over-ablating tissue and the need for re-ablating tissue can be reduced, thereby improving procedure safety, efficacy, and efficiency involving tissue ablation systems. These and other benefits of various embodiments of the present invention will become more apparent from the following descriptions and from the figures.

In the descriptions herein, certain specific details are set forth in order to provide a thorough understanding of various embodiments of the invention. However, one skilled in the art will understand that the invention may be practiced at a more general level without one or more of these details. In other instances, well known structures have not been shown or described in detail to avoid unnecessarily obscuring descriptions of various embodiments of the invention.

Any reference throughout this specification to “one embodiment”, “an embodiment”, “an example embodiment”, “an illustrated embodiment”, “a particular embodiment”, and the like means that a particular feature, structure or characteristic described in connection with the embodiment is included in at least one embodiment. Thus, any appearance of the phrase “in one embodiment”, “in an embodiment”, “in an example embodiment”, “in this illustrated embodiment”, “in this particular embodiment”, or the like in this specification is not necessarily always referring to one embodiment or a same embodiment. Furthermore, the particular features, structures or characteristics of different embodiments may be combined in any suitable manner to form one or more other embodiments.

Unless otherwise explicitly noted or required by context, the word “or” is used in this disclosure in a non-exclusive sense. In addition, unless otherwise explicitly noted or required by context, the word “set” is intended to mean one or more. For example, the phrase, “a set of objects” means one or more of the objects. In some embodiments, the word “subset” is intended to mean a set having the same or fewer elements of those present in the subset's parent or superset. In other embodiments, the word “subset” is intended to mean a set having fewer elements of those present in the subset's parent or superset. In this regard, when the word “subset” is used, some embodiments of the present invention utilize the meaning that “subset” has the same or fewer elements of those present in the subset's parent or superset, and other embodiments of the present invention utilize the meaning that “subset” has fewer elements of those present in the subset's parent or superset.

Further, the phrase “at least” is or may be used herein at times merely to emphasize the possibility that other elements may exist besides those explicitly listed. However, unless otherwise explicitly noted (such as by the use of the term “only”) or required by context, non-usage herein of the phrase “at least” nonetheless includes the possibility that other elements may exist besides those explicitly listed. For example, the phrase, ‘based at least on A’ includes A as well as the possibility of one or more other additional elements besides A. In the same manner, the phrase, ‘based on A’ includes A, as well as the possibility of one or more other additional elements besides A. However, the phrase, ‘based only on A’ includes only A. Similarly, the phrase ‘configured at least to A’ includes a configuration to perform A, as well as the possibility of one or more other additional actions besides A. In the same manner, the phrase ‘configured to A’ includes a configuration to perform A, as well as the possibility of one or more other additional actions besides A. However, the phrase, ‘configured only to A’ means a configuration to perform only A.

The word “device”, the word “machine”, the word “system”, and the phrase “device system” all are intended to include one or more physical devices or sub-devices (e.g., pieces of equipment) that interact to perform one or more functions, regardless of whether such devices or sub-devices are located within a same housing or different housings. However, it may be explicitly specified according to various embodiments that a device or machine or device system resides entirely within a same housing to exclude embodiments where the respective device, machine, system, or device system resides across different housings. The word “device” may equivalently be referred to as a “device system” in some embodiments, and the word “system” may equivalently be referred to as a “device system” in some embodiments.

Further, the phrase “in response to” may be used in this disclosure. For example, this phrase may be used in the following context, where an event A occurs in response to the occurrence of an event B. In this regard, such phrase includes, for example, that at least the occurrence of the event B causes or triggers or is a necessary precondition for the event A, according to various embodiments.

The phrase “thermal ablation” as used in this disclosure refers, in some embodiments, to an ablation method in which destruction of tissue occurs by hyperthermia (elevated tissue temperatures) or hypothermia (depressed tissue temperatures). Thermal ablation may include radiofrequency (“RF”) ablation, microwave ablation, or cryo-ablation by way of non-limiting example. Thermal ablation energy waveforms can take various forms. For example, in some thermal ablation embodiments, energy (e.g., RF energy) is provided in the form of a continuous waveform. In some thermal ablation embodiments, energy (e.g., RF energy) is provided in the form of discrete energy applications (e.g., in the form of a duty-cycled waveform).

The phrase “pulsed field ablation” (“PFA”) as used in this disclosure refers, in some embodiments, to an ablation method that employs high voltage pulse delivery in a unipolar or bipolar fashion in proximity to target tissue. In some embodiments, each high voltage pulse may be referred to as a discrete energy application. In some embodiments, a grouped plurality of high voltages pulses may be referred to as a discrete energy application. Each high voltage pulse can be a monophasic pulse including a single polarity, or a biphasic pulse including a first component having a first particular polarity and a second component having a second particular polarity opposite the first particular polarity. In some embodiments, the second component of the biphasic pulse follows immediately after the first component of the biphasic pulse. In some embodiments, the first and second components of the biphasic pulse are temporally separated by a relatively small time interval referred to as an intra-phase time period. In some embodiments, each high voltage pulse may include a multiphasic pulse, such as a triphasic pulse, that includes a first component having a first particular polarity, a second component having a second particular polarity opposite the first particular polarity, and a third component having a third particular polarity that is the same as the first particular polarity. In some embodiments, an intra-phase time period may separate successive components of the multiphasic pulse. The electric field applied by the high voltage pulses in PFA physiologically changes the tissue cells to which the energy is applied (e.g., puncturing or perforating the cell membrane to form various pores therein). If a lower field strength is established, the formed pores may close in time and cause the cells to maintain viability (e.g., a process sometimes referred to as reversible electroporation). If the field strength that is established is greater, then permanent, and sometimes larger, pores form in the tissue cells, the pores allowing loss of control of ion concentration gradients (both inwards and outwards) thereby eventually resulting in cell death (e.g., a process sometimes referred to as irreversible electroporation).

The word “fluid” as used in this disclosure should be understood to include any fluid that can be contained within a bodily cavity or can flow into or out of, or both into and out of a bodily cavity via one or more bodily openings positioned in fluid communication with the bodily cavity. In the case of cardiac applications, fluid such as blood will flow into and out of various intracardiac cavities (e.g., a left atrium or a right atrium).

The words “bodily opening” as used in this disclosure should be understood to include a naturally occurring bodily opening or channel or lumen; a bodily opening or channel or lumen formed by an instrument or tool using techniques that can include, but are not limited to, mechanical, thermal, electrical, chemical, and exposure or illumination techniques; a bodily opening or channel or lumen formed by trauma to a body; or various combinations of one or more of the above. Various elements having respective openings, lumens, or channels and positioned within the bodily opening (e.g., a catheter sheath) may be present in various embodiments. These elements may provide a passageway through a bodily opening for various devices employed in various embodiments.

The words “bodily cavity” as used in this disclosure should be understood to mean a cavity in a body. The bodily cavity may be a cavity or chamber provided in a bodily organ (e.g., an intracardiac cavity of a heart).

The word “tissue” as used in some embodiments in this disclosure should be understood to include any surface-forming tissue that is used to form a surface of a body or a surface within a bodily cavity, a surface of an anatomical feature or a surface of a feature associated with a bodily opening positioned in fluid communication with the bodily cavity. The tissue can include part, or all, of a tissue wall or membrane that defines a surface of the bodily cavity. In this regard, the tissue can form an interior surface of the cavity that surrounds a fluid within the cavity. In the case of cardiac applications, tissue can include tissue used to form an interior surface of an intracardiac cavity such as a left atrium or a right atrium. In some embodiments, the word tissue can refer to a tissue having fluidic properties (e.g., blood) and may be referred to as fluidic tissue.

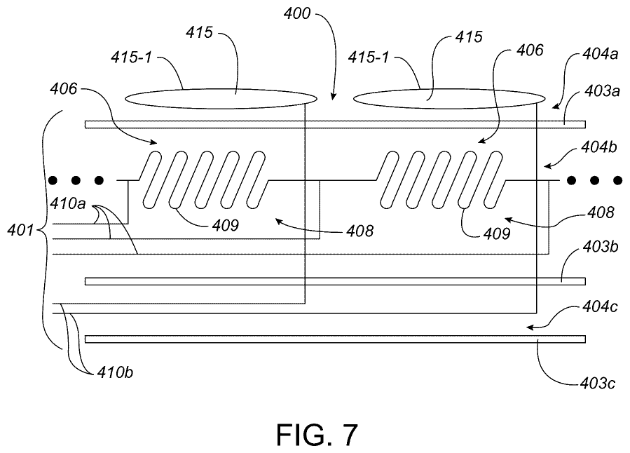

The term “transducer” as used in this disclosure should be interpreted broadly as any device capable of transmitting or delivering energy, distinguishing between fluid and tissue, sensing temperature, creating heat, ablating tissue, sensing, sampling or measuring electrical activity of a tissue surface (e.g., sensing, sampling or measuring intracardiac electrograms, or sensing, sampling or measuring intracardiac voltage data), stimulating tissue, providing location information (e.g., in conjunction with a navigation system), or any combination thereof. A transducer may convert input energy of one form into output energy of another form. Without limitation, a transducer may include an electrode that functions as, or as part of, a sensing device included in the transducer, an energy delivery device included in the transducer, or both a sensing device and an energy delivery device included in the transducer. A transducer may be constructed from several parts, which may be discrete components or may be integrally formed. In this regard, although transducers, electrodes, or both transducers and electrodes are referenced with respect to various embodiments, it is understood that other transducers or transducer elements may be employed in other embodiments. It is understood that a reference to a particular transducer in various embodiments may also imply a reference to an electrode, as an electrode may be part of the transducer as shown, e.g., at least with FIG. 7 discussed below.

In some embodiments, the term “activation” as used in this disclosure should be interpreted broadly as making active a particular function as related to various transducers disclosed in this disclosure. Particular functions may include, but are not limited to, tissue ablation (e.g., PFA or thermal ablation such as RF); sensing, sampling, or measuring electrophysiological activity (e.g., sensing, sampling, or measuring intracardiac electrogram information or sensing, sampling, or measuring intracardiac voltage data); sensing, sampling, or measuring temperature; and sensing, sampling, or measuring electrical characteristics (e.g., tissue impedance or tissue conductivity). For example, in some embodiments, activation of a tissue ablation function of a particular transducer is initiated by causing energy sufficient for tissue ablation from an energy source device system to be delivered to the particular transducer. Also, in this example, the activation can last for a duration of time concluding when the ablation function is no longer active, such as when energy sufficient for the tissue ablation is no longer provided to the particular transducer. In some contexts and embodiments, however, the word “activation” can merely refer to the initiation of the activating of a particular function, as opposed to referring to both the initiation of the activating of the particular function and the subsequent duration in which the particular function is active. In these contexts, the phrase or a phrase similar to “activation initiation” may be used.

In the following description, some embodiments of the present invention may be implemented at least in part by a data processing device system or a controller system configured by a software program. Such a program may equivalently be implemented as multiple programs, and some, or all, of such software program(s) may be equivalently constructed in hardware. In this regard, reference to “a program” should be interpreted to include one or more programs.

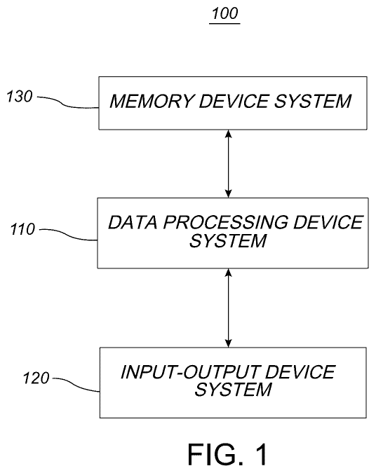

In some embodiments, the term “program” in this disclosure should be interpreted to include one or more programs including a set of instructions or modules that can be executed by one or more components in a system, such as a controller system or a data processing device system, in order to cause the system to perform one or more operations. The set of instructions or modules may be stored by any kind of memory device, such as those described subsequently with respect to the memory device system 130 or 330 shown in at least FIGS. 1, 5 and 6, respectively. In addition, this disclosure sometimes describes that the instructions or modules of a program are configured to cause the performance of a function. The phrase “configured to” in this context is intended to include at least (a) instructions or modules that are presently in a form executable by one or more data processing devices to cause performance of the function (e.g., in the case where the instructions or modules are in a compiled and unencrypted form ready for execution), and (b) instructions or modules that are presently in a form not executable by the one or more data processing devices, but could be translated into the form executable by the one or more data processing devices to cause performance of the function (e.g., in the case where the instructions or modules are encrypted in a non-executable manner, but through performance of a decryption process, would be translated into a form ready for execution). The word “module” can be defined as a set of instructions. In some instances, this disclosure describes that the instructions or modules of a program perform a function. Such descriptions should be deemed to be equivalent to describing that the instructions or modules are configured to cause the performance of the function.

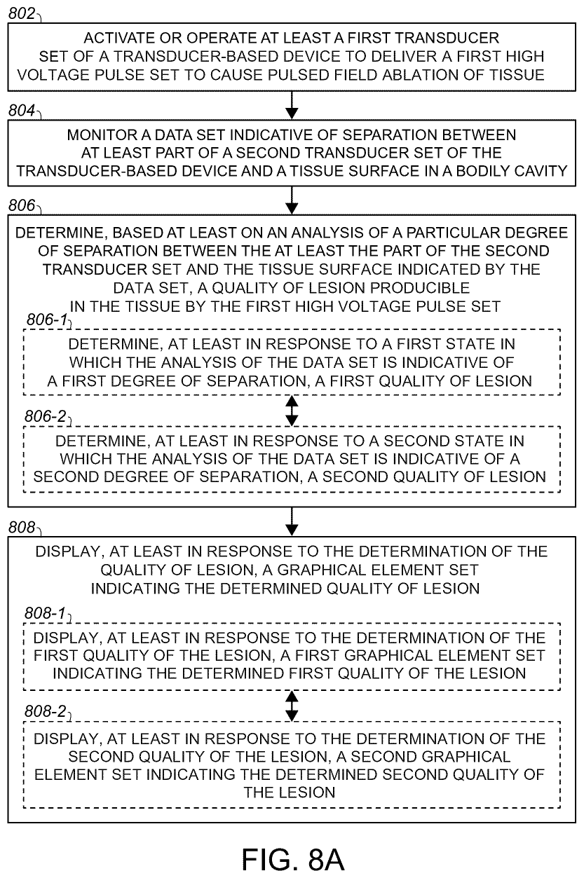

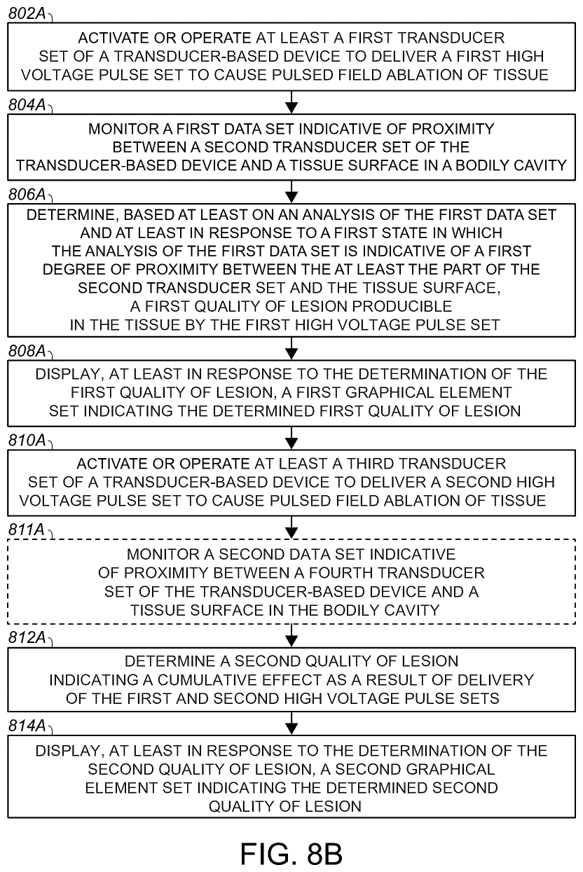

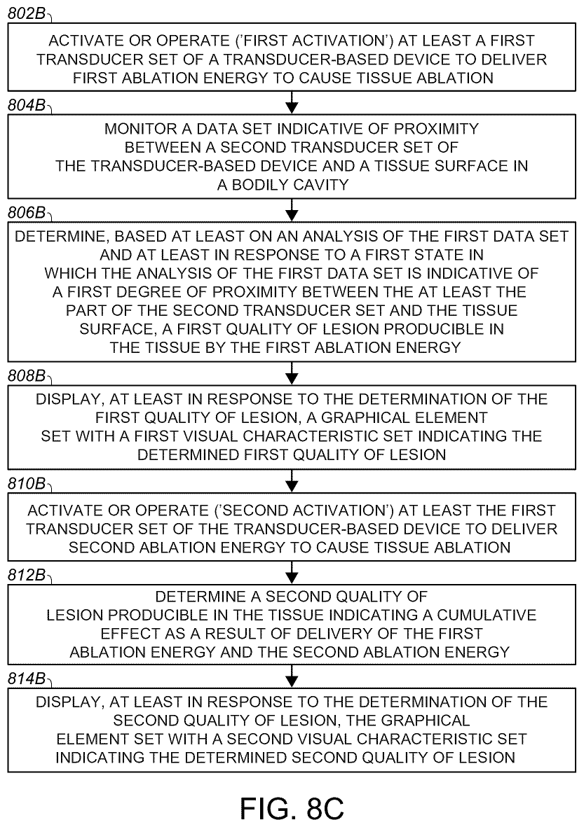

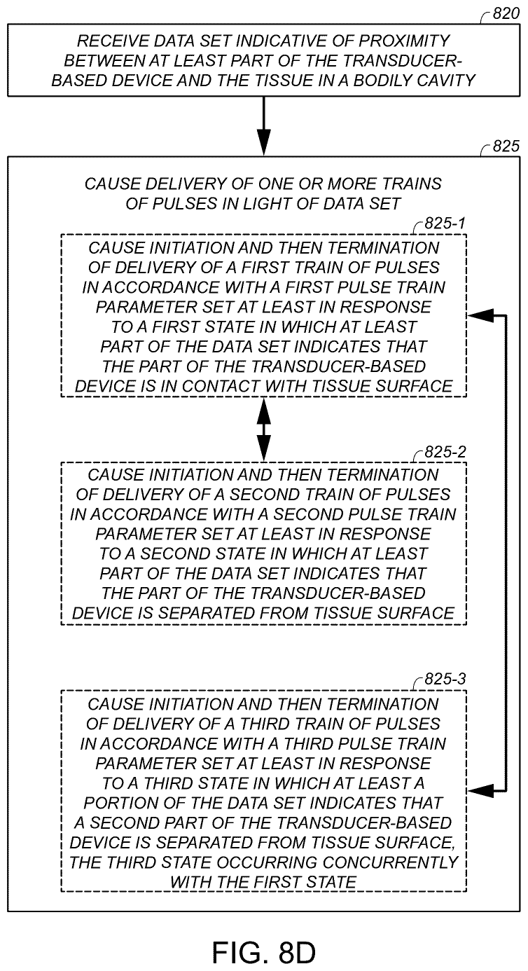

Example methods are described herein with respect to FIGS. 8A, 8B, and 8C. Such figures include blocks associated with actions, computer-executable instructions, or both, according to various embodiments. It should be noted that the respective instructions associated with any such blocks therein need not be separate instructions and may be combined with other instructions to form a combined instruction set. The same set of instructions may be associated with more than one block. In this regard, the block arrangement shown in each of the method figures herein is not limited to an actual structure of any program or set of instructions or required ordering of method tasks, and such method figures, according to some embodiments, merely illustrate the tasks that instructions are configured to perform, for example, upon execution by a data processing device system in conjunction with interactions with one or more other devices or device systems.

Each of the phrases “derived from” or “derivation of” or “derivation thereof” or the like may be used herein to mean to come from at least some part of a source, be created from at least some part of a source, or be developed as a result of a process in which at least some part of a source forms an input, according to various embodiments. For example, a data set derived from some particular portion of data may include at least some part of the particular portion of data, or may be created from at least part of the particular portion of data, or may be developed in response to a data manipulation process in which at least part of the particular portion of data forms an input. In some embodiments, a data set may be derived from a subset of the particular portion of data. In some embodiments, the particular portion of data is analyzed to identify a particular subset of the particular portion of data, and a data set is derived from the subset. In various ones of these embodiments, the subset may include some, but not all, of the particular portion of data. In some embodiments, changes in at least one part of a particular portion of data may result in changes in a data set derived at least in part from the particular portion of data.

In this regard, each of the phrases “derived from” or “derivation of” or “derivation thereof” or the like may be used herein merely to emphasize the possibility that such data or information may be modified or subject to one or more operations. For example, if a device generates first data for display, the process of converting the generated first data into a format capable of being displayed may alter the first data. This altered form of the first data may be considered a derivative or derivation of the first data. For instance, the first data may be a one-dimensional array of numbers, but the display of the first data may be a color-coded bar chart representing the numbers in the array. For another example, if the above-mentioned first data is transmitted over a network, the process of converting the first data into a format acceptable for network transmission or understanding by a receiving device may alter the first data. As before, this altered form of the first data may be considered a derivative or derivation of the first data. For yet another example, generated first data may undergo a mathematical operation, a scaling, or a combining with other data to generate other data that may be considered derived from the first data. In this regard, it can be seen that data is commonly changing in form or being combined with other data throughout its movement through one or more data processing device systems, and any reference to information or data herein is intended in some embodiments to include these and like changes, regardless of whether or not the phrase “derived from” or “derivation of” or “derivation thereof” or the like is used in reference to the information or data. As indicated above, usage of the phrase “derived from” or “derivation of” or “derivation thereof” or the like merely emphasizes the possibility of such changes. Accordingly, in some embodiments, the usage, non-usage, addition of, or deletion of the phrase “derived from” or “derivation of” or “derivation thereof” or the like should have no impact on the interpretation of the respective data or information. For example, the above-discussed color-coded bar chart may be considered a derivative of the respective first data or may be considered the respective first data itself, whether or not the phrase “derived from” or “derivation of” or “derivation thereof” or the like is used, according to some embodiments.

In some embodiments, the term “adjacent”, the term “proximate”, and the like refer at least to a sufficient closeness between the objects or events defined as adjacent, proximate, or the like, to allow the objects or events to interact in a designated way. For example, in the case of physical objects, if object A performs an action on an adjacent or proximate object B, objects A and B would have at least a sufficient closeness to allow object A to perform the action on object B. In this regard, some actions may require contact between the associated objects, such that if object A performs such an action on an adjacent or proximate object B, objects A and B would be in contact, for example, in some instances or embodiments where object A needs to be in contact with object B to successfully perform the action. In some embodiments, the term “adjacent”, the term “proximate”, and the like additionally or alternatively refer to objects or events that do not have another substantially similar object or event between them. For example, object or event A and object or event B could be considered adjacent or proximate (e.g., physically or temporally) if they are immediately next to each other (with no other object or event between them) or are not immediately next to each other but no other object or event that is substantially similar to object or event A, object or event B, or both objects or events A and B, depending on the embodiment, is between them. In some embodiments, the term “adjacent”, the term “proximate”, and the like additionally or alternatively refer to at least a sufficient closeness between the objects or events defined as adjacent, proximate, and the like, the sufficient closeness being within a range that does not place any one or more of the objects or events into a different or dissimilar region or time period, or does not change an intended function of any one or more of the objects or events or of an encompassing object or event that includes a set of the objects or events. Different embodiments of the present invention adopt different ones or combinations of the above definitions. Of course, however, the term “adjacent”, the term “proximate”, and the like are not limited to any of the above example definitions, according to some embodiments. In addition, the term “adjacent” and the term “proximate” do not have the same definition, according to some embodiments.

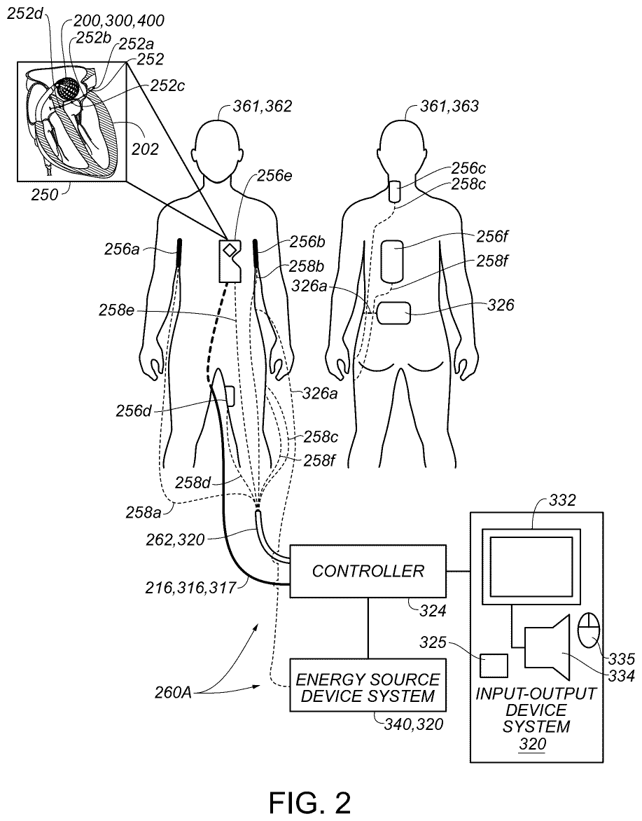

FIG. 1 schematically illustrates a portion of a transducer operation system or controller system thereof 100 that may be employed to at least operate, select, control, activate, or monitor a function or activation of a number of electrodes or ablation transducers (e.g., ablation transducers configured to cause thermal ablation or ablation transducers configured to cause PFA), according to some embodiments. The transducer operation system 100, may, according to various embodiments, determine a quality of a tissue lesion that is formable, or formed, in response to a delivery of tissue ablative energy. The system 100 includes a data processing device system 110, an input-output device system 120, and a processor-accessible memory device system 130. The processor-accessible memory device system 130 and the input-output device system 120 are communicatively connected to the data processing device system 110. According to some embodiments, various components, such as data processing device system 110, input-output device system 120, and processor-accessible memory device system 130, form at least part of a controller system (e.g., controller system 324 shown in FIGS. 2, 3, 5, and 6).

The data processing device system 110 includes one or more data processing devices that implement or execute, in conjunction with other devices, such as those in the system 100, various methods and functions described herein, including those described with respect to methods exemplified in FIGS. 8A, 8B, and 8C. Each of the phrases “data processing device”, “data processor”, “processor”, “controller”, “computing device”, “computer” and the like is intended to include any data or information processing device, such as a central processing unit (CPU), a control circuit, a desktop computer, a laptop computer, a mainframe computer, a tablet computer, a personal digital assistant, a cellular or smart phone, and any other device for processing data, managing data, or handling data, whether implemented with electrical, magnetic, optical, quantum, or biological components, or otherwise.

The memory device system 130 includes one or more processor-accessible memory devices configured to store one or more programs and information, including the program(s) and information needed to execute the methods or functions described herein, including those described with respect to method FIGS. 8A, 8B, 8C, and 8D. The memory device system 130 may be a distributed processor-accessible memory device system including multiple processor-accessible memory devices communicatively connected to the data processing device system 110 via a plurality of computers and/or devices. However, the memory device system 130 need not be a distributed processor-accessible memory system and, consequently, may include one or more processor-accessible memory devices located within a single data processing device or housing.

Each of the phrases “processor-accessible memory” and “processor-accessible memory device” and the like is intended to include any processor-accessible data storage device or medium, whether volatile or nonvolatile, electronic, magnetic, optical, or otherwise, including but not limited to, registers, hard disk drives, Compact Discs, DVDs, flash memories, ROMs, and RAMs. In some embodiments, each of the phrases “processor-accessible memory” and “processor-accessible memory device” is intended to include or be a processor-accessible (or computer-readable) data storage medium. In some embodiments, each of the phrases “processor-accessible memory” and “processor-accessible memory device” is intended to include or be a non-transitory processor-accessible (or computer-readable) data storage medium. In some embodiments, the processor-accessible memory device system 130 may be considered to include or be a non-transitory processor-accessible (or computer-readable) data storage medium or medium system. In some embodiments, the memory device system 130 may be considered to include or be a non-transitory processor-accessible (or computer-readable) storage medium system or data storage medium system including or consisting of one or more non-transitory processor-accessible (or computer-readable) storage or data storage mediums.

The phrase “communicatively connected” is intended to include any type of connection, whether wired or wireless, between devices, data processors, or programs between which data may be communicated. Further, the phrase “communicatively connected” is intended to include a connection between devices or programs within a single data processor or computer, a connection between devices or programs located in different data processors or computers, and a connection between devices not located in data processors or computers at all. In this regard, although the memory device system 130 is shown separately from the data processing device system 110 and the input-output device system 120, one skilled in the art will appreciate that the memory device system 130 may be located completely or partially within the data processing device system 110 or the input-output device system 120. Further in this regard, although the input-output device system 120 is shown separately from the data processing device system 110 and the memory device system 130, one skilled in the art will appreciate that such system may be located completely or partially within the data processing system 110 or the memory device system 130, for example, depending upon the contents of the input-output device system 120. Further still, the data processing device system 110, the input-output device system 120, and the memory device system 130 may be located entirely within the same device or housing or may be separately located, but communicatively connected, among different devices or housings. In the case where the data processing device system 110, the input-output device system 120, and the memory device system 130 are located within the same device, the system 100 of FIG. 1 may be implemented by a single application-specific integrated circuit (ASIC) in some embodiments.

The input-output device system 120 may include a mouse, a keyboard, a touch screen, another computer, or any device or combination of devices from which a desired selection, desired information, instructions, or any other data is input to the data processing device system 110. The input-output device system 120 may include a user-activatable control system that is responsive to a user action. The user-activatable control system may include at least one control element that may be activated or deactivated on the basis of a particular user action. The input-output device system 120 may include any suitable interface for receiving information, instructions or any data from other devices and systems described in various ones of the embodiments. In this regard, the input-output device system 120 may include various ones of other systems described in various embodiments. For example, the input-output device system 120 may include at least a portion of a transducer-based device system. The phrase “transducer-based device system” is intended to include one or more physical systems that include various transducers. The phrase “transducer-based device” is intended to include one or more physical devices that include various transducers. A thermal ablation or PFA device system that includes one or more transducers may be considered a transducer-based device or device system, according to some embodiments.

The input-output device system 120 also may include an image generating device system, a display device system, a speaker or audio output device system, a computer, a processor-accessible memory device system, a network-interface card or network-interface circuitry, or any device or combination of devices to which information, instructions, or any other data is output by the data processing device system 110. In this regard, the input-output device system 120 may include various other devices or systems described in various embodiments. The input-output device system 120 may include any suitable interface for outputting information, instructions, or data to other devices and systems described in various ones of the embodiments. If the input-output device system 120 includes a processor-accessible memory device, such memory device may, or may not, form part, or all, of the memory device system 130. The input-output device system 120 may include any suitable interface for outputting information, instructions, or data to other devices and systems described in various ones of the embodiments. In some embodiments, the input-output device system 120 may include a transducer-based device, as discussed above, and in some embodiments, the transducer-based device may act as a device or device system that provides information to, receives instructions or energy from, or both provides information to and receives instructions or energy from the data processing device system 110. In this regard, the input-output device system 120 may include various devices or systems described in various embodiments.

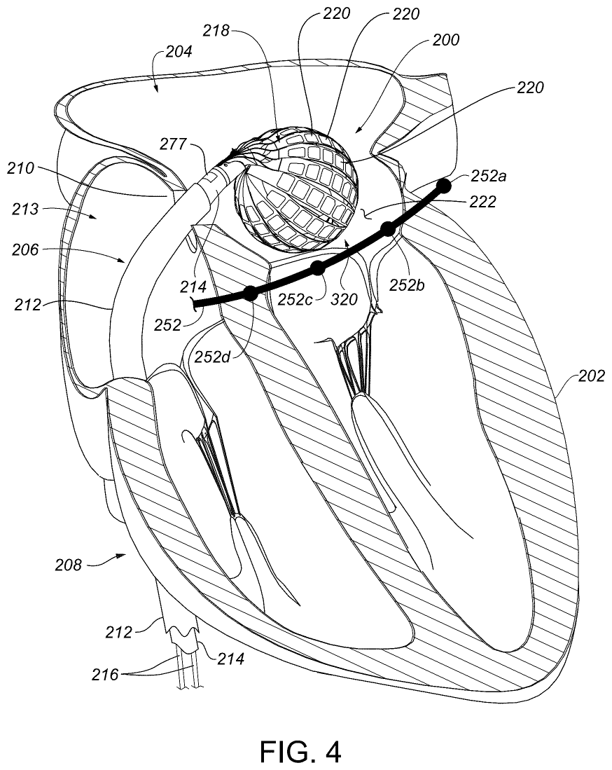

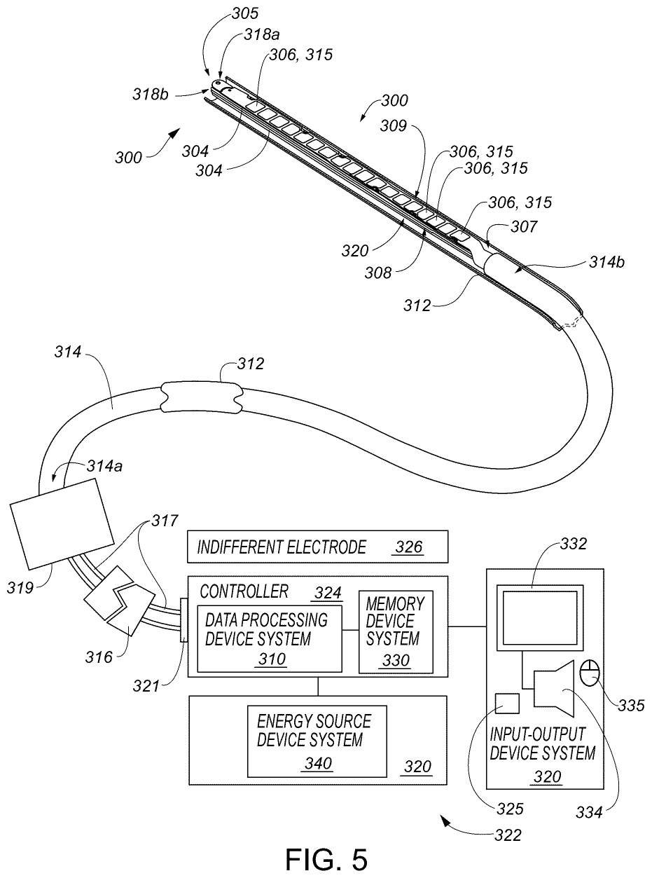

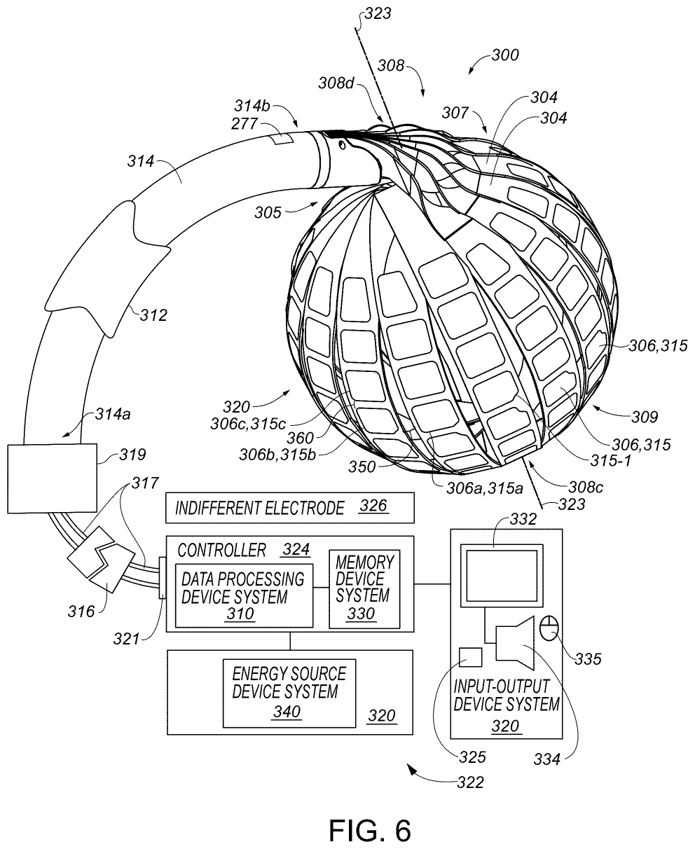

Various embodiments of transducer-based devices are described herein in this disclosure. Transducer-based devices or device systems described herein that include a catheter may also be referred to as catheter device systems, catheter devices or device systems, or catheter-based devices or device systems, according to various embodiments. Some of the described transducer-based devices are PFA devices or thermal ablation devices that are percutaneously or intravascularly deployed. Some of the described devices are movable between a delivery or unexpanded configuration (e.g., FIG. 5 discussed below, in some embodiments) in which a portion of the device is sized for passage through a bodily opening leading to a bodily cavity, and an expanded or deployed configuration (e.g., at least FIG. 4 or FIG. 6 discussed below, in some embodiments) in which the portion of the device has a size too large for passage through the bodily opening leading to the bodily cavity. An example of an expanded or deployed configuration, in some embodiments, is when the portion of the transducer-based device is in its intended-deployed-operational state, which may be inside the bodily cavity when, e.g., performing a therapeutic or diagnostic procedure for a patient, or which may be outside the bodily cavity when, e.g., performing testing, quality control, or other evaluation of the device. Another example of the expanded or deployed configuration, in some embodiments, is when the portion of the transducer-based device is being changed from the delivery configuration to the intended-deployed-operational state to a point where the portion of the device now has a size too large for passage through the bodily opening leading to the bodily cavity.

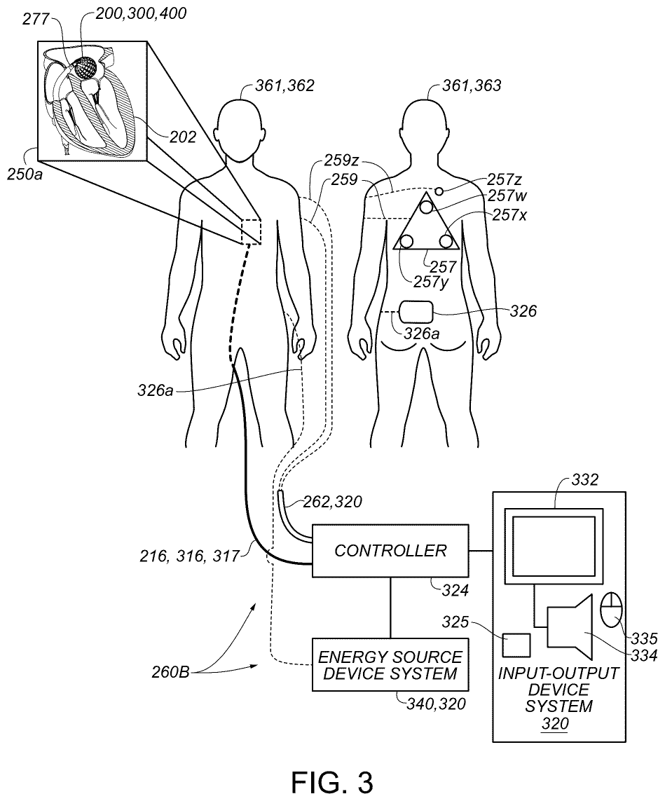

In some example embodiments, the transducer-based device includes transducers that sense characteristics (e.g., convective cooling, permittivity, force) that distinguish between fluid, such as a fluidic tissue (e.g., blood), and tissue forming an interior surface of the bodily cavity. Such sensed characteristics can allow a medical system to map the cavity, for example, using positions of openings or ports into and out of the cavity to determine a position or orientation (e.g., pose), or both of the portion of the device in the bodily cavity. In some example embodiments, the described systems employ a navigation system or electro-anatomical mapping system (e.g., as described below with respect to at least FIG. 2 or 3, according to some embodiments) including electromagnetic-based systems and electropotential-based systems to determine a positioning of at least a portion of a device in a bodily cavity or to determine a degree of proximity of a portion of a device to a tissue surface. In some example embodiments, the described devices are part of a tissue ablation system capable of ablating tissue in a desired pattern within the bodily cavity using various techniques (e.g., via thermal ablation, PFA, etc., according to various embodiments).

In some example embodiments, the transducer-based devices are capable of sensing various cardiac functions (e.g., electrophysiological activity including intracardiac voltages). In some example embodiments, the devices are capable of providing stimulation (e.g., electrical stimulation) to tissue within the bodily cavity. Electrical stimulation may include pacing.

FIG. 2 includes a partially schematic representation of some particular implementations of a device location tracking system 260A (which may be referred to as a catheter navigation system 260A in some embodiments) implementing an electric-field-based location system, according to various example embodiments. According to some embodiments, the navigation system 260A is part of various tissue ablation systems described in this disclosure. FIG. 2 illustrates a controller 324, which may, at least in part, be a particular implementation of the data processing device system 110 shown in FIG. 1, in some embodiments. In this regard, the controller 324 may also be a particular implementation of at least part of the memory device system 130 shown in FIG. 1, in some embodiments. Illustrated in FIG. 2 is an input-output device system 320 communicatively connected to the controller 324 and may include a display device system 332, a mouse 335 or other pointing device system, a speaker device system 334 or other audio output device system, or a sensing device system 325, according to various embodiments. Possible contents of the input-output device system 320 are discussed in more detail below. The input-output device system 320 may be a particular implementation of the input-output device system 120 shown in FIG. 1, in some embodiments. FIG. 2 also illustrates, in cut-out illustration window 250, a catheter or transducer-based device 200, 300, or 400, discussed in more detail below, which may be communicatively connected to the controller 324 via electrical conductors 216, cable 316, electrical leads 317 (discussed in more detail below; see, e.g., at least FIGS. 4-6), or a combination thereof, according to various embodiments. According to various embodiments, catheter or transducer-based device 200, 300, or 400 may form part of a tissue ablation system. The electrical conductors 216, cable 316, or electrical leads 317 may reside, at least in part, within a catheter shaft 214 or 314 or within a catheter sheath 212 or 312 discussed in more detail below (see, e.g., at least FIGS. 4-6). The catheter or transducer-based device 200, 300, or 400 may include one or more transducers (discussed in more detail below; see, e.g., at least FIGS. 4-7) and may be included in the input-output device system 320, according to some embodiments. In FIG. 2, the catheter or transducer-based device 200, 300, or 400 is illustrated via cut-out illustration window 250 within a heart 202 of a patient 361, although the catheter or transducer-based device 200, 300, or 400 may instead be operated outside of any living being, e.g., in a quality-control, training, or testing environment. The single patient 361 is illustrated in two parts in FIG. 2 merely to concurrently show the front side 362 and the back side 363 of the patient 361, although the various connections to the controller 324 are only fully shown via the illustrated front side 362 of the patient 361 for purposes of clarity. The portion of the electrical conductors 216, cable 316, or electrical leads 317 that is outside the patient 361 is illustrated in thick solid line in FIG. 2, and the portion of the electrical conductors 216, cable 316, or electrical leads 317 that is inside the patient 361 is illustrated in thick broken line in FIG. 2.

Also illustrated in FIG. 2 is an energy source device system 340 communicatively connected to the controller 324. The energy source device system 340 may be part of the input-output device system 320 and may be configured to provide energy to the transducers of the catheter or transducer-based device 200, 300, or 400 for sensing, tissue ablation, or both, according to various embodiments and as discussed in more detail below. According to various embodiments, energy delivered to catheter or transducer-based device 200, 300, or 400 for tissue ablation may be configured to cause thermal ablation or PFA. Electrode 326, shown on the lower back of the back side 363 of patient 361 in FIG. 2, for example, may be communicatively connected to energy source device system 340 via conductor 326a. Electrode 326 may be placed externally on the body of the patient 361, according to some embodiments. Electrode 326 may be an indifferent electrode, which may facilitate the performance of impedance sensing or ablation, particularly monopolar or blended monopolar-bipolar ablation, according to some embodiments. Indifferent electrode 326 is discussed in more detail below.

FIG. 2 also illustrates electrodes 256a, 256b, 256c, 256d, 256e, and 256f that are placed externally on the body of the patient 361, according to some embodiments. The electrodes 256a, 256b, 256c, 256d, 256e, and 256f may be included in the input-output device system 320 and may be communicatively connected to the controller 324 via respective electrical conductors 258a, 258b, 258c, 258d, 258e, and 258f partially inside cable 262, according to some embodiments. Although respective electrical conductors 258a, 258b, 258c, 258d, 258e, and 258f are shown within a same cable 262 for clarity of illustration, one or more of such electrical conductors may be in separate cables. According to some embodiments, electrodes 256a, 256b, 256c, 256d, 256e, and 256f are configured to generate electric fields that enable the controller 324 to determine, in conjunction with corresponding sensing performed by transducers of the catheter or transducer-based device 200, 300, or 400, X, Y, and Z coordinate axis location information of the catheter or transducer-based device 200, 300, or 400 within the heart 202 of the patient 361 or in a quality-control, training, or testing environment. In particular, electrodes 256a and 256b (a first pair of electrodes) may be configured to generate a first electric field at a first frequency or frequency range that the transducers of the catheter or transducer-based device 200, 300, or 400 are configured to sense as, e.g., respective X-axis locations of the respective transducers. Similarly, electrodes 256c and 256d (a second pair of electrodes) may be configured to generate a second electric field at a second frequency or frequency range that the transducers of the catheter or transducer-based device 200, 300, or 400 are configured to sense as, e.g., respective Y-axis locations of the respective transducers. Similarly, electrodes 256e and 256f (a third pair of electrodes) may be configured to generate a third electric field at a third frequency or frequency range that the transducers of the catheter or transducer-based device 200, 300, or 400 are configured to sense as, e.g., respective Z-axis locations of the respective transducers. The first, second, and third frequencies or frequency ranges may be mutually exclusive, according to some embodiments. In some embodiments, the first, second, and third electric fields may have a same frequency or frequency range and be time-multiplexed in coordination with time-multiplexed sensing by the transducers of the catheter or transducer-based device 200, 300, or 400, to facilitate repeated sequential sensing of respective X, Y, and Z-axis locations of the respective transducers.

Electric field strength sensed by one or more transducers of the catheter or transducer-based device 200, 300, or 400 may be evaluated by the controller 324 or its data processing device system 310 (shown in FIG. 5) to determine location information including respective three-dimensional X, Y, and Z-axis locations of the transducers with respect to the first, second, and third electric fields and with respect to reference device 252 (shown as including reference electrodes 252a, 252b, 252c, and 252d, although fewer or more may be provided), according to some embodiments. The reference device 252 (see, e.g., cut-out illustration window 250 in FIG. 2 or see, e.g., FIG. 4 for more detail) may be located within the body of the patient 361, preferably in a location that keeps its positioning relatively stable, such as in the coronary sinus, to factor out transitory movements of the transducer(s) of the transducer-based device 200, 300, or 400 due, e.g., to the beating of the heart. The one or more reference electrodes (e.g., reference electrodes 252a, 252b, 252c, and 252d) of the reference device 252 may be configured to also sense electric field strength of the first, second, and third electric fields, and the three-dimensional location of the transducer-based device 200, 300, or 400 is determined by the controller 324 or its data processing device system 310 with respect to the reference device 252 based on the measurements made by the transducers of the catheter or transducer-based device 200, 300, or 400 and the measurements made by the reference electrodes (e.g., reference electrodes 252a, 252b, 252c, and 252d) of the reference device 252, according to some embodiments.

The measurements made by the transducers of the catheter or transducer-based device 200, 300, or 400, the measurements made by the reference electrodes of the reference device 252 (or reference device 257z discussed in more detail below with respect to FIG. 3, in some embodiments), or both may, in some embodiments, provide at least part of location information (in the form of a location signal set in some embodiments) indicating locations of at least part of a transducer-based device 200, 300, or 400 in a bodily cavity or relative to a tissue surface in a bodily cavity. In some embodiments, even if (i) the measurements made by the transducers of the catheter or transducer-based device 200, 300, or 400, (ii) the measurements made by the reference electrodes of the reference device 252 (or reference device 257z in some embodiments), or both (i) and (ii) indicate locations of at least part of the transducer-based device 200, 300, or 400 with respect to an absolute reference frame associated with locations derived solely from the three-dimensional X, Y, and Z-axes, such location information may indicate (e.g., by derivation or by combination with tissue contact sensing information provided by electrodes of the transducer-based device in some embodiments) locations of at least part of the transducer-based device 200, 300, or 400 relative to a tissue surface in the bodily cavity, according to some embodiments. In some embodiments, measurements made by the transducers of the catheter or transducer-based device 200, 300, or 400 derived relatively to the measurements made by the reference electrodes of the reference device 252 or reference device 257z may indicate locations of at least part of a transducer-based device 200, 300, or 400 relative to a tissue surface in a bodily cavity. In this regard, a reference, such as reference device 252 or reference device 257z may, according to various embodiments, help define a coordinate frame that moves with an organ that includes the bodily cavity (e.g., movement of the organ resulting from the cardiac cycle or pulmonary cycle), and measurements made in this coordinate frame may accordingly indicate locations of at least part of a transducer-based device 200, 300, or 400 relative to a tissue surface in a bodily cavity, according to some embodiments. However, in some embodiments, the locations of at least part of a catheter or transducer-based device may be indicated by location information without necessarily being relative to a tissue surface. U.S. Pat. No. 5,697,377, issued on Dec. 16, 1997 (Wittkampf), provides examples of how to determine a three-dimensional location of a catheter (e.g., an electrode position).

In this regard, FIG. 2 illustrates a device location tracking system 260A, according to some embodiments. According to various embodiments, the device location tracking system 260A may include at least some of: one or more external electrodes (e.g., electrodes 256a, 256b, 256c, 256d, 256e, and 256f), one or more reference electrodes (e.g., reference electrodes 252a, 252b, 252c, 252d of reference device 252), the controller 324 or data processing system 310 or 110, the transducers of the catheter (e.g., transducer-based device 200, 300, or 400), and a display device system (e.g., display device system 332). In some embodiments, the display device system, the catheter, the device location tracking system, or a combination thereof may be included as part of an input-output device system (e.g., input-output device system 320 or input-output device system 120) of a transducer operation system.

FIG. 3 includes a partially schematic representation of some particular implementations of a device location tracking system 260B (sometimes referred to as catheter navigation system 260B) implementing a magnetic-field-based location system, according to various example embodiments. In this regard, FIG. 3 corresponds to FIG. 2, except that a magnetic-field-based location system is illustrated instead of an electric-field-based location system. Instead of electrodes 256a, 256b, 256c, 256d, 256e, and 256f, FIG. 3 illustrates three magnetic field generation sources 257w, 257x, and 257y, such as coils, each of which respectively generates a magnetic field, according to some embodiments. The magnetic field generation sources 257w, 257x, and 257y may be integrally formed within a package or frame 257 located beneath the patient 361. Magnetic field sources 257w, 257x, and 257y may respectively be connected to the controller 324 via a set of one or more conductors 259, which may or may not be located within the same cable 262. Similarly, the reference device 257z may be connected to the controller 324 via a set of one or more conductors 259z, which may or may not be included in conductor set 259, and which may or may not be located within the same cable 262. The transducer-based device 200, 300, or 400 may include one or more magnetic field transducers 277 (shown in the cut-out illustration window 250a in FIG. 3) configured to sense the strengths of the magnetic fields generated by magnetic field sources 257w, 257x, and 257y. As with some embodiments associated with FIG. 2, the magnetic field sources 257w, 257x, and 257y need not generate different magnetic fields, but may instead generate the same magnetic fields in a time-multiplexed manner that are sensed in sequence over time by the one or more magnetic field transducers 277. In some embodiments, the one or more magnetic field transducers 277 may sense the magnetic field strengths with respect to a reference device 257z, which may be akin to the reference device 252 in the electric field context of FIG. 2. With the three magnetic field strengths detected by the one or more magnetic field transducers 277 for a given time or time period, the distance(s) between the one or more transducers 277 and the magnetic field generation sources 257w, 257x, and 257y may be determined, and the three-dimensional location of the one or more transducers 277 may be determined according to triangulation as per some embodiments. With the location of the one or more transducers 277 in three-dimensional space known, and the geometry of the transducer-based device 200, 300, or 400 (e.g., including the locations of the transducers on the transducer-based device) relative to transducers 277 also known, the locations of the transducers of the transducer-based device 200, 300, or 400 for the given time or time period may be determined, according to some embodiments. U.S. Patent Application Publication No. 2007/0265526 (Govari et al.), published on Nov. 15, 2007, provides examples of how to determine a three-dimensional location of a catheter in a magnetic-field-based system.

FIG. 4 is a representation of a transducer-based device 200 useful in investigating or treating a bodily organ, for example, a heart 202, according to some embodiments. In some embodiments, the transducer-based device 200 may form part of a tissue ablation system. In some embodiments, all or part of the transducer-based device 200 may form part or all of a transducer operation system.

Transducer-based device 200 can be percutaneously or intravascularly inserted into a portion of the heart 202, such as an intracardiac cavity, like left atrium 204. In this example, the transducer-based device 200 is, or is part of a catheter 206 inserted via the inferior vena cava 208 and penetrating through a bodily opening in transatrial septum 210 from right atrium 213. In other embodiments, other paths may be taken.

Catheter 206 includes an elongated flexible rod or shaft member appropriately sized to be delivered percutaneously or intravascularly. Various portions of catheter 206 may be steerable. For example, a structure 218 supporting transducers 220 may be controlled via various manipulations to advance outwardly, to retract, to rotate clockwise, to rotate counterclockwise, and to have a particular deployment plane orientation (e.g., a plane in which the structure progresses from a delivery configuration (e.g., described below with respect to at least FIG. 5) to or at least toward a deployed configuration (e.g., described below with respect to at least FIG. 6), according to some embodiments. One or more other portions of the transducer-based device 200 may be steerable. For example, a catheter sheath 212, which encompasses or surrounds at least part of an elongate shaft member 214 to which the structure 218 is physically coupled, may be steerable. In some embodiments, the sheath 212 may be controlled via various manipulations to advance outwardly, retract, rotate clockwise, rotate counterclockwise, bend, release a bend, and have a particular bending plane orientation, according to various embodiments.

Catheter 206 may include one or more lumens. The lumen(s) may carry one or more communications or power paths, or both. For example, the lumens(s) may carry one or more electrical conductors 216 (two shown). Electrical conductors 216 provide electrical connections to transducer-based device 200 and transducers 220 thereof that are accessible externally from a patient in which the transducer-based device 200 is inserted.

Transducer-based device 200 may include a frame or structure 218 which assumes an unexpanded configuration for delivery to left atrium 204, according to some embodiments, such frame or structure supporting transducers 220. Structure 218 is expanded (e.g., shown in a deployed or expanded configuration in FIG. 4) upon delivery to left atrium 204 to position a plurality of transducers 220 (three called out in FIG. 4) proximate the interior surface formed by tissue 222 of left atrium 204. In some embodiments, at least some of the transducers 220 may be configured to sense a physical characteristic of a fluid (e.g., blood) or tissue 222, or both, that may be used to determine tissue contact. In some embodiments, at least some of the transducers 220 may be configured to selectively ablate portions of the tissue 222. For example, some of the transducers 220 may be configured to ablate a pattern around the bodily openings, ports, or pulmonary vein ostia, for instance, to reduce or eliminate the occurrence of atrial fibrillation. In some embodiments, at least some of the transducers 220 are configured to ablate cardiac tissue. In some embodiments, at least some of the transducers 220 are configured to sense or sample intracardiac voltage data or sense or sample intracardiac electrogram data. In some embodiments, at least some of the transducers 220 are configured to sense or sample intracardiac voltage data or sense or sample intracardiac electrogram data while at least some of the transducers 220 are concurrently ablating cardiac tissue. In some embodiments, at least one of the sensing or sampling transducers 220 is or are provided by at least one of the ablating transducers 220. In some embodiments, at least a first one of the transducers 220 senses or samples intracardiac voltage data or intracardiac electrogram data at a location at least proximate a tissue location ablated by at least a second one of the transducers 220. In some embodiments, the first one of the transducers 220 is other than the second one of the transducers 220.

FIGS. 5 and 6 include a catheter device system (e.g., a portion thereof shown schematically) that includes a transducer-based device 300, according to some embodiments. All or part of such catheter system may be all, or part of, a tissue ablation system, according to various embodiments. All or part of such catheter system may be all, or part of, a transducer-operation system, according to various embodiments. The transducer-based device 300 may be the same as the transducer-based device 200, although different sizes, numbers of transducers, or types of transducer-based devices, such as balloon catheters, may be implemented. In this regard, transducer-based device 300 includes a plurality of elongate members 304 (not all of the elongate members are called out in FIGS. 5 and 6) and a plurality of transducers 306 (not all of the transducers are called out in FIGS. 5 and 6; some of the transducers 306 are called out in FIG. 6 as 306a, 306b, and 306c). FIG. 5 includes a representation of a portion of the transducer-based device 300 in a delivery or unexpanded configuration. FIG. 6 includes a representation of a portion of the transducer-based device 300 in an expanded or deployed configuration. It is noted that, for clarity of illustration, all of the elongate members shown in FIG. 6 are not represented in FIG. 5. The plurality of transducers 306 is positionable within a bodily cavity, such as with the transducer-based device 200. For example, in some embodiments, the transducers 306 are able to be positioned in a bodily cavity by movement into, within, or into and within the bodily cavity, with or without a change in a configuration of the plurality of transducers 306. In some embodiments, the transducers of the plurality of transducers 306 are arranged to form a two- or three-dimensional distribution, grid, or array of the transducers capable of mapping, ablating, or stimulating an inside surface of a bodily cavity or lumen without requiring mechanical scanning. As shown, for example, in FIG. 5, the plurality of transducers 306 are arranged in a distribution receivable in a bodily cavity. In FIGS. 5 and 6, each of at least some of transducers 306 includes a respective electrode 315 (not all of the electrodes 315 are called out in FIGS. 5 and 6).

The elongate members 304 are arranged in a frame or structure 308 that is selectively movable between an unexpanded or delivery configuration (e.g., as shown in FIG. 5) and an expanded or deployed configuration (e.g., as shown in at least FIG. 6) that may be configured to position elongate members 304 against a tissue surface within the bodily cavity or position the elongate members 304 in the vicinity of the tissue surface or in proximity to the tissue surface. In some embodiments, structure 308 has a size in the unexpanded or delivery configuration suitable for delivery through a bodily opening (e.g., via catheter sheath 312) to the bodily cavity. In various embodiments, catheter sheath 312 typically includes a length sufficient to allow the catheter sheath to extend between a location at least proximate a bodily cavity into which the structure 308 is to be delivered and a location outside a body comprising the bodily cavity. In some embodiments, structure 308 has a size in the expanded or deployed configuration too large for delivery through a bodily opening (e.g., via catheter sheath 312) to the bodily cavity. The elongate members 304 may form part of a flexible circuit structure (e.g., also known as a flexible printed circuit board (PCB) circuit, examples of which are described with respect to FIG. 7, below). The elongate members 304 may include a plurality of different material layers. Each of the elongate members 304 may include a plurality of different material layers. The structure 308 may include a shape memory material, for instance, Nitinol. The structure 308 may include a metallic material, for instance, stainless steel, or non-metallic material, for instance, polyimide, or both a metallic and non-metallic material by way of non-limiting example. The incorporation of a specific material into structure 308 may be motivated by various factors including the specific requirements of each of the unexpanded or delivery configuration and expanded or deployed configuration, the required position or orientation (e.g., pose), or both of structure 308 in the bodily cavity, the requirements for successful ablation of a desired pattern, or the effect that the material may have on electric or magnetic fields to be sensed by the device (e.g., by one or more transducers 306 or one or more magnetic field transducers 277).

FIG. 7 is a schematic side elevation view of at least a portion of a transducer-based device 400 that includes a flexible circuit structure 401 that is configured to provide a plurality of transducers 406 (two called out), according to some embodiments. In some embodiments, the flexible circuit structure 401 may form part of a structure (e.g., structure 308) that is selectively movable between a delivery configuration sized for percutaneous delivery and expanded or deployed configurations sized too large for percutaneous delivery. In some embodiments, the flexible circuit structure 401 may be located on, or form at least part of, a structural component (e.g., elongate member 304) of a transducer-based device system.

The flexible circuit structure 401 may be formed by various techniques including flexible printed circuit techniques. In some embodiments, the flexible circuit structure 401 includes various layers including flexible layers 403a, 403b, and 403c (e.g., collectively flexible layers 403). In some embodiments, each of flexible layers 403 includes an electrical insulator material (e.g., polyimide). One or more of the flexible layers 403 may include a different material than another of the flexible layers 403. In some embodiments, the flexible circuit structure 401 includes various electrically conductive layers 404a, 404b, and 404c (collectively electrically conductive layers 404) that are interleaved with the flexible layers 403. In some embodiments, each of the electrically conductive layers 404 is patterned to form various electrically conductive elements. For example, electrically conductive layer 404a may be patterned to form a respective electrode 415 of each of the transducers 406. Electrodes 415 may have respective electrode edges 415-1 that form a periphery of an electrically conductive surface associated with the respective electrode 415. It is noted that other electrodes employed in other embodiments may have electrode edges arranged to form different electrode shapes (for example, as shown by electrode edge 315-1 in FIG. 6, according to some embodiments).

Electrically conductive layer 404b is patterned, in some embodiments, to form respective temperature sensors 408 for each of the transducers 406, as well as various leads 410a arranged to provide electrical energy to the temperature sensors 408. In some embodiments, each temperature sensor 408 includes a patterned resistive member 409 (two called out) having a predetermined electrical resistance. In some embodiments, each resistive member 409 includes a metal having relatively high electrical conductivity characteristics (e.g., copper). In some embodiments, electrically conductive layer 404c is patterned to provide portions of various leads 410b arranged to provide an electrical communication path to electrodes 415. In some embodiments, leads 410b are arranged to pass through vias in flexible layers 403a and 403b to connect with electrodes 415. Although FIG. 7 shows flexible layer 403c as being a bottom-most layer, some embodiments may include one or more additional layers underneath flexible layer 403c, such as one or more structural layers, such as a steel or composite layer. These one or more structural layers, in some embodiments, are part of the flexible circuit structure 401 and can be part of, e.g., elongate member 304. In some embodiments, the one or more structural layers may include at least one electrically conductive surface (e.g., a metallic surface) exposed to blood flow. In addition, although FIG. 7 shows only three flexible layers 403a-403c and only three electrically conductive layers 404a-404c, it should be noted that other numbers of flexible layers, other numbers of electrically conductive layers, or both, may be included.

In some embodiments, electrodes 415 are employed to selectively deliver thermal ablation energy (e.g., RF energy) to various tissue structures within a bodily cavity (e.g., an intracardiac cavity or chamber in some embodiments). In some embodiments, the thermal energy may be delivered in the form of a continuous waveform. In some embodiments, the thermal ablation energy may be delivered in the form of plurality of discrete energy applications (e.g., in the form of a duty-cycled waveform). The thermal energy delivered to the tissue may be delivered to cause monopolar thermal tissue ablation, bipolar thermal tissue ablation, or blended monopolar-bipolar thermal tissue ablation by way of non-limiting examples.

In some embodiments, electrodes 415 are employed to selectively deliver discrete energy applications in the form of PFA high voltage pulses to various tissue structures within a bodily cavity (e.g., an intracardiac cavity or chamber in some embodiments). The PFA high voltage pulses delivered to the tissue structures may be sufficient for ablating portions of the tissue structures. The PFA high voltage pulses delivered to the tissue may be delivered to cause monopolar pulsed field tissue ablation, bipolar pulsed field tissue ablation, or blended monopolar-bipolar pulsed field tissue ablation by way of non-limiting examples. The energy that is delivered by each high voltage pulse may be dependent upon factors including the electrode location, size, shape, relationship with respect to another electrode (e.g., the distance between adjacent electrodes that deliver the PFA energy), the presence, or lack thereof, of various materials between the electrodes, the degree of electrode-to-tissue contact, and other factors. In some cases, a maximum ablation depth resulting from the delivery of high voltage PFA pulses by a relatively smaller electrode is typically shallower than that of a relatively larger electrode.

In some embodiments, each electrode 415 is configured to sense or sample an electric potential in the tissue proximate the electrode 415 at a same or different time than delivering energy sufficient for tissue ablation. In some embodiments, each electrode 415 is configured to sense or sample intracardiac voltage data in the tissue proximate the electrode 415. In some embodiments, each electrode 415 is configured to sense or sample data in the tissue proximate the electrode 415 from which an electrogram (e.g., an intracardiac electrogram) may be derived. In some embodiments, each resistive member 409 is positioned adjacent a respective one of the electrodes 415. In some embodiments, each of the resistive members 409 is positioned in a stacked or layered array with a respective one of the electrodes 415 to form a respective one of the transducers 406. In some embodiments, the resistive members 409 are connected in series to allow electrical current to pass through all of the resistive members 409. In some embodiments, leads 410a are arranged to allow for a sampling of electrical voltage in between each resistive member 409. This arrangement allows for the electrical resistance of each resistive member 409 to be accurately measured. The ability to accurately measure the electrical resistance of each resistive member 409 may be motivated by various reasons including determining temperature values at locations at least proximate the resistive member 409 based at least on changes in the resistance caused by temperature changes caused by convective cooling effects (e.g., as provided by blood flow). The temperature dependent resistance data can thus be correlated to the degree of presence of the flow between the electrode 415 and tissue, thereby allowing the degree of contact between the electrode 415 and the tissue to be determined. Other methods of detecting transducer-to-tissue contact or degrees of transducer-to-tissue contact may be employed according to various example embodiments.

Referring to FIGS. 5 and 6, transducer-based device 300 can communicate with, receive power from, or be controlled by a transducer operation system 322 according to some embodiments. In some embodiments, the transducer operation system 322 represents one or more particular implementations of the system 100 illustrated in FIG. 1. In some embodiments, elongate members 304 include transducers 306 that are communicatively connected to a data processing device system 310 via electrical connections running within elongate shaft member 314 that are communicatively connected to one or more of electrical leads 317 (e.g., control leads, data leads, power leads or any combination thereof) within elongated cable 316 (only a portion of which is shown in FIGS. 5 and 6 to reveal other structures) terminating at a connector 321 or other interface. The leads 317 may correspond to the electrical conductors 216 in FIG. 4 in some embodiments and, although only two leads 317 are shown for clarity, more may be present. The transducer operation system 322 may include a controller 324 that includes the data processing device system 310 (e.g., which may be a particular implementation of data processing device system 110 from FIG. 1) and a memory device system 330 (e.g., which may be a particular implementation of the memory device system 130 from FIG. 1) that stores data and instructions that are executable by the data processing device system 310 to process information received from transducer-based device 300 or to control operation of transducer-based device 300, for example, by activating various selected transducers 306 to ablate tissue and control a user interface (e.g., of input-output device system 320) according to various embodiments. Controller 324 may include one or more controllers.

Transducer operation system 322 includes an input-output device system 320 (e.g., which may be a particular implementation of the input-output device system 120 from FIG. 1) communicatively connected to the data processing device system 310 (e.g., via controller 324 in some embodiments). Input-output device system 320 may include a user-activatable control that is responsive to a user action. Input-output device system 320 may include one or more user interfaces or input/output (I/O) devices, for example, one or more display device systems 332, speaker device systems 334, one or more keyboards, one or more mice (e.g., mouse 335), one or more joysticks, one or more track pads, one or more touch screens or other transducers to transfer information to, from, or both to and from a user, for example, a care provider such as a physician or technician. For instance, output from a mapping process may be displayed by a display device system 332. Input-output device system 320 may include one or more user interfaces or input/output (I/O) devices, for example, one or more display device systems 332, speaker device systems 334, keyboards, mice, joysticks, track pads, touch screens or other transducers employed by a user to indicate a particular selection or series of selections of various graphical information. Input-output device system 320 may include a sensing device system 325 configured to detect various characteristics including, but not limited to, at least one of tissue characteristics (e.g., electrical characteristics such as tissue impedance, electric potential of a tissue surface, tissue conductivity, tissue type, tissue thickness) and thermal characteristics such as temperature. In this regard, the sensing device system 325 may include one, some, or all, of the transducers 306 (or 220 in FIG. 4 or 406 of FIG. 7) of the transducer-based device 300, including the internal components of such transducers shown in FIG. 7, such as the electrodes 415 and temperature sensors 408.

Transducer operation system 322 may also include an energy source device system 340 including one or more energy source devices connected to transducers 306. In this regard, although FIGS. 5 and 6 show a communicative connection between the energy source device system 340 and the controller 324 (and its data processing device system 310), the energy source device system 340 may also be connected to the transducers 306 via a communicative connection that is independent of the communicative connection with the controller 324 (and its data processing device system 310). For example, the energy source device system 340 may receive control signals via the communicative connection with the controller 324 (and its data processing device system 310), and, in response to such control signals, deliver energy to, receive energy from, or both deliver energy to and receive energy from one or more of the transducers 306 via a communicative connection with such transducers 306 (e.g., via one or more communication lines through catheter body or elongate shaft member 314, elongated cable 316 or catheter sheath 312) that does not pass through the controller 324. In this regard, the energy source device system 340 may provide results of its delivering energy to, receiving energy from, or both delivering energy to and receiving energy from one or more of the transducers 306 to the controller 324 (and its data processing device system 310) via the communicative connection between the energy source device system 340 and the controller 324.

The energy source device system 340 may, for example, be connected to various selected transducers 306 to selectively provide energy in the form of electrical current or power, light or low temperature fluid to the various selected transducers 306 to cause ablation of tissue. The energy source device system 340 may, for example, selectively provide energy in the form of electrical current to various selected transducers 306 to facilitate measuring of a temperature characteristic, an electrical characteristic, or both at a respective location at least proximate each of the various transducers 306. The energy source device system 340 may include various electrical current sources or electrical power sources as energy source devices. In some embodiments, an indifferent electrode 326 is provided to receive at least a portion of the energy transmitted by at least some of the transducers 306. Consequently, although not shown in FIGS. 5 and 6, the indifferent electrode 326 may be communicatively connected to the energy source device system 340 via one or more communication lines in some embodiments. In addition, although shown separately in each of FIGS. 5 and 6, indifferent electrode 326 may be considered part of the energy source device system 340 in some embodiments. In various embodiments, indifferent electrode 326 is positioned on an external surface (e.g., a skin-based surface) of a body that comprises the bodily cavity into which at least transducers 306 are to be delivered.

It is understood that input-output device system 320 may include other systems. In some embodiments, input-output device system 320 may optionally include energy source device system 340, transducer-based device 300 or both energy source device system 340 and transducer-based device 300 by way of non-limiting example. Input-output device system 320 may include the memory device system 330 in some embodiments.

Structure 308 may be delivered and retrieved via a catheter member, for example, a catheter sheath 312. In some embodiments, a structure provides expansion and contraction capabilities for a portion of the medical device (e.g., an arrangement, distribution, or array of transducers 306). The transducers 306 may form part of, be positioned or located on, mounted or otherwise carried on the structure and the structure may be configurable to be appropriately sized to slide within catheter sheath 312 in order to be deployed percutaneously or intravascularly. FIGS. 5 and 6 show one embodiment of such a structure. In some embodiments, each of the elongate members 304 includes a respective distal end 305 (only one called out in each of FIGS. 5 and 6), a respective proximal end 307 (only one called out in each of FIGS. 5 and 6) and a respective intermediate portion 309 (only one called out in each of FIGS. 5 and 6) positioned between the proximal end 307 and the distal end 305. The respective intermediate portion 309 of each elongate member 304 includes a first or front surface 318a that is positionable to face an interior tissue surface within a bodily cavity and a second or back surface 318b opposite across a thickness of the intermediate portion 309 from the front surface 318a. In some embodiments, each of the elongate members 304 is arranged front surface 318a-toward-back surface 318b in a stacked array during an unexpanded or delivery configuration similar to that described in International Publication No. WO 2012/100184, published Jul. 26, 2012 (Fernando Lopes et al.) and International Publication No. WO 2012/100185, published Jul. 26, 2012 (Fernando Lopes et al.). In many cases, a stacked array allows the structure 308 to have a suitable size for percutaneous or intravascular delivery. In some embodiments, the elongate members 304 are arranged to be introduced into a bodily cavity distal end 305 first. An elongate shaft member 314 is configured to deliver structure 308 through catheter sheath 312, according to some embodiments. According to various embodiments, the elongate shaft member 314 includes a proximal end portion 314a and a distal end portion 314b, the distal end portion 314b physically coupled to structure 308. According to various embodiments, the elongate shaft member 314 may include a length to position distal end portion 314b (and structure 308 in some embodiments) at a desired location within a patient's body while maintaining the proximal end portion 314a at a location outside the patient's body. In some embodiments, the proximal end portion 314a may be coupled to a housing 319. Housing 319 may include or enclose various actuators that may be configured to manipulate various portions of the catheter, including, but not limited to, (a) portions of the elongate shaft member 314, (b) portions of structure 308, or both (a) and (b). According to various embodiments, housing 319 may take the form of a handle that is directly manipulable by a user. U.S. Pat. No. 9,452,016, issued Sep. 27, 2016 (Moisa et al.), provides possible examples of a housing and accompanying actuators that may be utilized as housing 319.

The transducers 306 can be arranged in various distributions or arrangements in various embodiments. In some embodiments, various ones of the transducers 306 are spaced apart from one another in a spaced apart distribution in the delivery configuration shown in FIG. 5. In some embodiments, various ones of the transducers 306 are arranged in a spaced apart distribution in the deployed configuration shown in at least FIG. 6. In some embodiments, various pairs of transducers 306 are spaced apart with respect to one another. In some embodiments, various regions of space are located between various pairs of the transducers 306. For example, in FIG. 6, the transducer-based device 300 includes at least a first transducer 306a, a second transducer 306b, and a third transducer 306c (all collectively referred to as transducers 306). In some embodiments, each of the first, the second, and the third transducers 306a, 306b, and 306c are adjacent transducers in the spaced apart distribution. In some embodiments, the first and the second transducers 306a, 306b are located on different elongate members 304, while the second and the third transducers 306b, 306c are located on a same elongate member 304. In some embodiments, a first region of space 350 is between the first and the second transducers 306a, 306b. In various embodiments, a first region of space 350 is between the respective electrodes 315a, 315b of the first and the second transducers 306a, 306b. In some embodiments, the first region of space 350 is not associated with any physical portion of structure 308. In some embodiments, a second region of space 360 associated with a physical portion of device 300 (e.g., a portion of an elongate member 304) is between the second and the third transducers 306b, 306c (and their respective electrodes 315b, 315c). In various embodiments, the second region of space 360 is between the respective electrodes 315b, 315c of the second and the third transducers 306b, 306c. In some embodiments, each of the first and the second regions of space 350, 360 does not include a transducer of transducer-based device 300. In some embodiments, each of the first and the second regions of space 350, 360 does not include any transducer. It is noted that other embodiments need not employ a group of elongate members 304 as employed in the illustrated embodiment. For example, other embodiments may employ a structure having one or more surfaces, at least a portion of the one or more surfaces defining one or more openings in the structure. In these embodiments, a region of space not associated with any physical portion of the structure may extend over at least part of an opening of the one or more openings.

In some embodiments, the transducers of the plurality of transducers (e.g., at least a group of the transducers 306) may be circumferentially arranged about an axis (e.g., axis 323, FIG. 6) of the structure 308 at least in the state in which the structure 308 is in the deployed configuration, the axis intersecting both the first portion of the structure (e.g., portion 308c in FIG. 6) and the second portion of the structure (e.g., portion 308d in FIG. 6) in the state in which the structure 308 is in the deployed configuration. According to various embodiments, portions 308c and 308d may each include a respective polar region of the structure 308 in the deployed configuration. In other example embodiments, other structures may be employed to support or carry transducers of a transducer-based device such as a transducer-based catheter. For example, an elongated catheter member may be used to distribute the transducers in a linear or curvilinear array. Basket catheters or balloon catheters may be used to distribute the transducers in a two-dimensional or three-dimensional array. According to some embodiments, a system is provided that may include an input-output device system (e.g., 120, 320) that may, in some embodiments, include a catheter that includes a plurality of transducers (e.g., transducers 220, 306, 406). The catheter may include the catheter body to which the plurality of transducers (or the structure on which the transducers reside) is physically coupled (e.g., catheter 206 and elongate shaft member 314). In some embodiments, the catheter may also include other components such as catheter sheath 312. According to various embodiments, different portions of the catheter are manipulable to in turn manipulate various ones of the plurality of transducers (e.g., transducers 220, 306, 406) into various degrees of proximity with a tissue wall within a patient's body (e.g., patient 361). According to various embodiments, various degrees of proximity between a portion of a transducer-based device and tissue may include (a) various degrees of contact between the portion of the transducer-based device and the tissue, or (b) various degrees of separation between the portion of the transducer-based device and the tissue. A particular type of proximity (e.g., contact or separation) and the particular amount of proximity that occurs in a particular procedure may vary based on various factors including the manipulation capability of the catheter, various anatomical constraints, and the skill of the health care provider. In thermal ablation procedures (e.g., an RF ablation procedure), contact between a thermal ablation transducer and tissue is desired at the time that thermal ablative energy is delivered by the thermal ablation transducer. In cardiac applications, when the thermal ablation transducer is separated from the tissue while delivering ablative energy, the blood is directly exposed to this energy and may become thermally denatured and form thermal coagulum which may pose a safety risk. Further, thermal ablation such as RF ablation loses efficacy rapidly with increasing degrees of transducer-to-tissue separation. In cardiac applications in which blood is adjacent tissue, the pattern of joule heating under the RF ablation transducer (e.g., electrode) is typically unchanged in a state in which the transducer is separated from the tissue as compared to a state in which the transducer contacts the tissue, and the RF ablation mechanism that occurs primarily involves trying to heat up the blood in order to warm up the tissue via contact with the warmed blood. In cardiac RF ablation applications, joule heating is typically concentrated in the first one millimeter of the tissue in the vicinity of the periphery of the electrode. In the presence of any sort of blood flow, however, the generated heat that is deposited gets carried away, and it becomes a struggle to get the heat to accumulate to levels required for efficacious (e.g., transmural) lesions. Thermal ablation typically requires sustained thermal buildup to get to temperatures required for thermal ablation, and even relatively low levels of blood flow can result in little tissue warming when there is no contact between the thermal ablation transducer and the tissue. The same effect applies when the proximity between the thermal ablation transducer and the tissue involves relatively low levels of tissue contact. In this regard, the thermal ablation transducer may include an electrode, and when just part of the electrode touches the tissue, the contacting part of the electrode may be considered in simplified terms to behave more like a smaller electrode and the lesion depth is diminished accordingly. In contrast, pulsed field ablation (PFA) typically does not cause tissue ablation via thermal buildup. Rather, PFA causes tissue ablation via irreversible electroporation, which requires that the tissue be exposed to the generated electric fields. Although these electric fields may be affected by an impedance difference between tissue and blood, the quality of the lesions that are formed is generally more robust to loss of immediate transducer-to-tissue contact. In the limit, if tissue and blood are considered to have the same conductivity, theory may indicate that for every additional millimeter of transducer-to-tissue separation that is experienced, approximately a millimeter of lesion depth reduction occurs for reasonable amounts of transducer-to-tissue separation (e.g., 1-3 mm) The same effect applies when the proximity between the PFA transducer and the tissue involves relatively low levels of tissue contact. The present inventors have delivered PFA energy to tissue with relatively high levels of transducer-to-tissue contact (i.e., as assessed with the aid the flow-based contact techniques described below in this disclosure) to achieve lesion depths on the order of 4-5 mm, and have delivered PFA energy to tissue with relatively lower levels of transducer-to-tissue contact (i.e., again as assessed with the aid of the flow-based contact techniques described in this disclosure) that are associated with lower lesion depth (i.e., on the order of 3 mm) In this regard, it is estimated that PFA energy may be delivered to cause a lesion in tissue with varying degrees of separation from (i.e., no contact with) the tissue. Of course, the range of separation is dependent on PFA energy levels delivered and desired lesion depth, as well as on transducer, blood, and tissue characteristics, so different embodiments may have different ranges of acceptable transducer/tissue separation when performing PFA. It can be important in some contexts for the transducer operation system performing PFA to determine and communicate to a user a quality of a tissue lesion formed by PFA particularly when the ablating transducer(s) are separated from the tissue, in some embodiments. Further in this regard, various embodiments of the present invention, as described herein and with the figures, determine such a quality of lesion as a function of transducer/tissue separation and manipulate one or more graphical elements to communicate to a user such determined lesion quality.

In some embodiments, a degree of proximity between a transducer set and tissue is a degree of contact between the transducer set and the tissue. According to various embodiments, different degrees of contact are associated with varying depths into tissue in which a particular transducer may be pressed or inserted. In some embodiments, a transducer may include a particular tissue-contacting portion configured to contact tissue (e.g., an electrode surface), and different degrees of contact are associated with varying amounts of the particular tissue-contacting portion of that transducer that contact the tissue. In some embodiments, the particular tissue-contacting portion of the transducer forms an entirety of a transducer surface that is configured to contact tissue. For example, in some embodiments, the tissue-contacting portion may form an entirety of a surface of a substantially planar electrode (e.g., electrode 315), the entirety of the surface configured to contact tissue. In some embodiments, the particular tissue-contacting portion of the transducer forms some but not all of a transducer surface that is configured to contact tissue. For example, in some embodiments, the tissue-contacting portion may form some but not all of a cylindrically shaped electrode that in use is configured to have a first part contact tissue while concurrently having a second part not contacting tissue.

Various contact sensing systems and methods may be executed to determine the degree of transducer-to-tissue contact including, by way of non-limiting example, techniques including sensing impedance, sensing permittivity, sensing the presence or absence of flow of a fluid (e.g., a bodily fluid), or sensing contact force or pressure. U.S. Pat. No. 8,906,011, issued Dec. 9, 2014 (Gelbart et al.), describes example transducer sensing techniques employed by various contact sensing systems. In some embodiments, the tissue-contacting portion of the transducer itself directly senses the degree of tissue contact. In some embodiments, a portion of the transducer other than the tissue-contacting portion of the transducer is configured to sense the degree of contact between the tissue wall and the tissue-contacting portion of the transducer. In some embodiments, the tissue-contacting portion of the transducer is provided by an electrode. In some embodiments, a second transducer is employed to determine transducer-to-tissue contact of a first transducer. In some embodiments, the second transducer does not form part of a transducer-based device that includes the first transducer. For example, the first transducer may be a transducer 306 of transducer-based device 300, and the second transducer may belong to the device location tracking system 260A or 260B or an optical or ultrasonic or other device, discussed in more detail below, that helps determine the location of the transducer-based device 300, e.g., with respect to a tissue wall or a computer-generated model of the bodily cavity.

In some embodiments, a degree of proximity between a transducer set and tissue is a degree of separation (i.e., having no contact) between the transducer set and the tissue. Unlike a degree of contact between a transducer set and tissue which is, indicates, or is associated with some amount of transducer set-to-tissue contact, a degree of separation between a transducer set and tissue is, indicates, or is associated with some amount of transducer set-to-tissue separation in some embodiments. In some embodiments, the degree of separation between a transducer set and tissue is, indicates, or is associated with an amount of separation between the transducer set and the tissue. In some embodiments, the degree of separation between a transducer set and tissue is, indicates, or is associated with an amount of separation between a location representative of the transducer set and the tissue. For instance, such location representative of the transducer set may be a geographic center or centroid of an area or volume encompassing outer boundaries of the one or more transducers in the transducer set, in some embodiments. In some embodiments, the transducer set is a spatial distribution of multiple transducers (e.g., transducer 206, 306, or 406, in some embodiments), and the degree of separation between the transducer set and tissue is, indicates, or is associated with one or more locations in the spatial distribution. For example, a geographic center or centroid (e.g., an average physical or virtual location) associated with the spatial distribution may be determined based on locations of each of the multiple transducers, the centroid corresponding to a location in the spatial distribution. In some embodiments, the degree of separation between a transducer set and tissue is, indicates, or is associated with a respective amount of separation between each of at least one transducer in the transducer set and the tissue. In some embodiments, the degree of separation between a transducer set and tissue is, indicates, or is associated with a respective amount of separation between each transducer in the transducer set and the tissue. In some embodiments, the degree of separation between a transducer set and tissue is, indicates, or is associated with a respective amount of separation between each of at least one transducer in the transducer set and the tissue. If the transducer set includes only one transducer, the degree of separation between the transducer set and tissue is, indicates, or is associated with a respective amount of separation between the transducer and the tissue.

According to some embodiments, various methods may be executed to determine the degree of transducer set-to-tissue separation including, by way of non-limiting example, techniques including sensing impedance, sensing permittivity, sensing the presence or absence of flow of a fluid (e.g., a bodily fluid). Another method that may be executed in some embodiments to determine the degree of transducer set-to-tissue separation techniques may include an electrogram-based technique in which a detected sharpness of a recorded electrogram may act as an indicator of transducer-to-tissue proximity including transducer set-to-tissue separation. Another method that may be executed in some embodiments to determine the degree of transducer set-to-tissue separation may include optical-based techniques which can be employed to determine transducer-to-tissue separation with either wide or narrow band imaging. Yet another method that may be executed in some embodiments to determine the degree of transducer set-to-tissue separation may include acoustic based techniques. For example, ultrasound sensors may be used to determine the degree of transducer set-to-tissue separation in some embodiments. According to various embodiments, various sensors employed to determine a degree of transducer set-to-tissue separation may be referred to as proximity sensors, since at least in some cases, they may be able to determine both degree of contact and degree of separation in some embodiments. It is noted that several methods of determining a degree of transducer set-to-tissue separation as described above as well as other methods may be employed to determine a degree of transducer set-to-tissue contact, and as such may in some embodiments, be referred to as methods of determining degrees of transducer set-to-tissue proximity According to various embodiments, various sensors employed to determine a degree of transducer set-to-tissue separation, or a degree of transducer set-to-tissue contact may be referred to as proximity sensors. Other methods of determining degrees of transducer set-to-tissue proximity or transducer set-to-tissue separation may include the use of a device location tracking system or navigation system (for example, systems shown in FIG. 2 or 3). As described in further detail below, a device location tracking system or navigation system may be employed to provide or facilitate determination of location information associated with a particular transducer set and location information associated with a tissue surface, and, accordingly, based on the location information, determine degrees of transducer set-to-tissue proximity or transducer set-to-tissue separation. FIGS. 8A-8D illustrate respective programmed configurations of a data processing device system (e.g., data processing device system 110 or 310), according to some embodiments of the present invention. For example, a programmed configuration may be implemented by the data processing device system being communicatively connected to an input-output device system (e.g., input-output device system 120 or 320) and a memory device system (e.g., memory device system 130 or 330), and being configured by a program stored by the memory device system at least to perform one or more actions (e.g., such as at least one, more, or all of the actions described in any one or more of FIGS. 8A-8D or otherwise herein). In some embodiments in which the one or more of the programmed configurations illustrated in FIGS. 8A-8D actually is or are executed at least in part by the data processing device system, such actual execution may be considered a respective method executed by the data processing device system. In this regard, FIGS. 8A-8D may be considered to represent one or more methods in some embodiments and, for ease of communication, such one or more methods may be referred to simply as ‘the method of FIG. 8A’, ‘the method of FIG. 8B’, ‘the method of FIG. 8C’, ‘the method of FIG. 8D’, and the like. The blocks shown in each of FIGS. 8A-8D may be associated with computer-executable instructions of a program that configures the data processing device system to perform the actions described by the respective blocks. According to various embodiments, not all of the actions or blocks shown in each of FIGS. 8A-8D are required, and different orderings of the actions or blocks shown in each of FIGS. 8A-8D may exist. In this regard, in some embodiments, a subset of the blocks shown in each of FIGS. 8A-8D or additional blocks may exist. In some embodiments, a different sequence of various ones of the blocks in each of FIGS. 8A-8D or actions described therein may exist.

In some embodiments, a memory device system (e.g., memory device system 130 or 330, e.g., a computer-readable medium system) stores the program(s) represented by each of FIGS. 8A-8D, and, in some embodiments, the memory device system is communicatively connected to the data processing device system as a configuration thereof. In this regard, in various example embodiments, a memory device system (e.g., memory device system 130, 330) is communicatively connected to a data processing device system (e.g., data processing device systems 110 or 310) and stores a program executable by the data processing device system to configure or cause the data processing device system to execute various actions described by, or otherwise associated with, the blocks illustrated in each of FIGS. 8A-8D for performance of some or all of the corresponding method(s) via interaction with at least, for example, a transducer-based device (e.g., transducer-based device devices 200, 300, or 400, in some embodiments). In this regard, in various example embodiments, a memory device system (e.g., memory device system 130 or 330, in some embodiments) is communicatively connected to a data processing device system (e.g., data processing device systems 110 or 310, in some embodiments) and stores a program executable by the data processing device system to configure or cause the data processing device system to execute various actions described by, or otherwise associated with, the blocks illustrated in each of FIGS. 8A-8D for performance of some or all of the corresponding method(s) via interaction with at least, for example, a device location tracking system or navigation system (e.g., as described above in this disclosure with respect to FIGS. 2 and 3). In some embodiments, the program may include instructions configured to perform, or cause to be performed, various ones of the block actions described by or otherwise associated with one or more, or all, of the blocks illustrated in each of FIGS. 8A-8D for performance of some, or all, of the corresponding method(s).

FIG. 8A shows configurations of the data processing device system to behave differently in association with different states, respectively referred to at least by broken-line blocks 806-1 and 806-2 within block 806, and respectively referred to at least by broken-line blocks 808-1, 808-2 within block 808. In this regard, either or both of the states and corresponding actions set forth in blocks 806-1, 806-2 and blocks 808-1, 808-2 may actually occur or be executed by the data processing device system (e.g., as in a method) in some embodiments, and, in the case where both states and corresponding actions referred to by blocks 806-1, 806-2 or blocks 808-1, 808-2 actually occur or are executed by the data processing device system, they may occur in any order, as illustrated by the double-headed arrow shown in FIG. 8A between blocks 806-1, 806-2, and also between blocks 808-1,808-2, according to various embodiments.

In FIG. 8A, according to some embodiments, block 802 represents a configuration of the data processing device system (e.g., data processing device system 110 or 310) (e.g., configured according to a program) to cause, via an input-output device system (e.g., input-output device system 120 or 320), activation or operation of at least a first transducer set of a transducer-based device (e.g., 200, 300, or 400) to deliver a first high voltage pulse set to cause pulsed field ablation of tissue. According to various embodiments, each transducer (e.g., transducer 206, 306, or 406) is configured to deliver high voltage pulses configured to cause pulsed field ablation of tissue. According to some embodiments, the activation or operation of at least a first transducer set is configured to deliver the first high voltage pulse set to cause monopolar PFA. According to some embodiments, the activation or operation of at least a first transducer set is configured to deliver the first high voltage pulse set to cause bipolar PFA.

According to some embodiments, the data processing device system (e.g., data processing device system 110 or 310) is configured to cause a transducer set, such as the first transducer set or another transducer set described herein, to cause the delivery of a high voltage pulse set, such as the first high voltage pulse set or another high voltage pulse set described herein, as the delivery of a particular high voltage pulse set that is made of or includes a determined or predetermined number of high voltage pulses. In some embodiments, the pulses in a high voltage pulse set are successively arranged with a constant (or regular) pulse-to-pulse spacing. In some embodiments, the pulses in a high voltage pulse set are arranged with a constant (or regular) pulse frequency. In some embodiments, a first high voltage pulse set (e.g., per at least block 802) is distinguished from another high voltage pulse set due to a spacing between a last pulse in the first high voltage pulse set and a first pulse in the other high voltage pulse set (or vice versa) being greater (e.g., at least greater than two times, three times, ten times, thirty times, sixty times, one hundred times, or more in various embodiments) than a between-pulse spacing between pulses within the first high voltage pulse set or within the other high voltage pulse set, in various embodiments. However, inter-pulse set spacing need not change between multiple high voltage pulse sets, such that multiple high voltage pulse sets may collectively form a continuous high voltage pulse superset, in some embodiments. In some embodiments, the boundaries of a high voltage pulse set need not be determined by or need not be determined solely by inter-pulse spacing characteristics, but, e.g., may be determined, at least in part, by energy density delivered, such as an amount of high voltage energy delivered per unit time, according to some embodiments. For example, regardless of the internal characteristics of the pulses of a high voltage pulse set, the boundaries of the high voltage pulse set may be determined by a minimum amount of high voltage energy delivered per millisecond or some other appropriate time period appropriate for defining a PFA high voltage pulse set. However, in some embodiments, multiple high voltage pulse sets may have a same amount of energy delivered per unit time. In some embodiments, a high voltage pulse set forms part, but not all, of another high voltage pulse set, such as in a case in which the first high voltage pulse set (e.g., per at least block 802) is part of an uninterrupted train of high voltage pulses, such that the first high voltage pulse set and a second and possibly additional high voltage pulse set collectively form the uninterrupted high voltage pulse train. In this case, the uninterrupted high voltage pulse train may itself be considered a high voltage pulse set made of multiple subsets of high voltage pulses, and of the multiple subsets of high voltage pulses, the first high voltage pulse set may be one of them. In instances where multiple high voltage pulse sets do not have a characteristic described above that would otherwise separate them into distinct high voltage pulse sets, the multiple high voltage pulse sets may be collectively considered to be at least part of an uninterrupted high voltage pulse set, superset, or train, according to some embodiments.

In some embodiments, at least one transducer (e.g., transducer 206, 306, or 406, in some embodiments) may be configured to perform other functions in addition to delivering pulsed field ablation, as may be with the first transducer set referred to at least in block 802. For example, at least one transducer in the first transducer set may be configured to sense electrophysiological information or perform other functions as described above in this disclosure.

In FIG. 8A, according to some embodiments, block 804 represents a configuration of the data processing device system (e.g., data processing device system 110 or 310) (e.g., configured according to a program) to cause, via the input-output device system (e.g., input-output device system 120 or 320), monitoring of a data set indicative of separation or a degree of separation (i.e., non-contact) between a second transducer set of the transducer-based device (e.g., 200, 300, or 400) and a tissue surface in a bodily cavity. Various examples of a degree of separation between a transducer set and tissue have been provided above, according to some embodiments, and the data set may be indicative of such separation between the second transducer set and the tissue surface of the bodily cavity. According to some embodiments, the second transducer set is one or more transducers, such as transducer 206, 306, 406, or 277 in some embodiments. In some embodiments, each transducer in the second transducer set is the same as or similar to each transducer in the first transducer set. For example, in some embodiments, each transducer in the second transducer set may be a transducer (e.g., transducer 206, 306, or 406, in some embodiments) configured to deliver pulsed field ablation, and may be configured to perform some other particular function (e.g., one or more various sensing functions) that a transducer in the first transducer set is configured to perform. In some embodiments, each transducer in the second transducer set is other than each transducer in the first transducer set. In some embodiments, each transducer in the second transducer set is different, or is configured to perform a different activity than each transducer in the first transducer set. For example, the first transducer set may be configured to deliver pulsed field ablation, while the second transducer set may include a transducer that is not configured to deliver pulsed field ablation energy, in some embodiments, or any form of tissue ablative energy in some embodiments and, e.g., may instead be configured to perform a sensing function. For instance, in some embodiments, a magnetic field sensing transducer set (e.g., 277) is not typically configured to deliver ablative energy and may be included in the second transducer set. In such a case, a data set provided by the magnetic field sensing transducer (e.g., 277) to the data processing device system (e.g., 110, 310) for monitoring per block 804 may indicate the positional information of the magnetic field sensing transducer set 277 in three-dimensional space, in some embodiments. In some embodiments, the data processing device system may also have, e.g., via a model of the bodily cavity discussed in more detail below, the location of the tissue surface in that three-dimensional space. With, e.g., both the location of the magnetic field sensing transducer 277 and the location of the bodily cavity in a common three-dimensional space, a degree of separation between the magnetic field sensing transducer 277 and the tissue surface in the bodily cavity may be determined by the data processing device system (e.g., 110, 310), in some embodiments. And, in some embodiments, with the spatial relationship between various transducers (206, 306, 406) with respect to the magnetic field sensing transducer set 277 also known by the data-processing device system, the degree of separation between any transducer of the transducer-based device and the tissue surface may be determined by the data-processing device system from the location of the magnetic field sensing transducer set 277, in some embodiments. However, other manners of determining degree of separation between a transducer and a tissue surface may be utilized, according to various embodiments.

In some embodiments, the second transducer set is at least a part of the first transducer set. For instance, in some embodiments, the second transducer set and the first transducer set may be the same entity/entities, such that the same transducers that deliver the first high voltage pulse set per block 802 may also be the same transducers whose distance(s) from the tissue surface are monitored per block 804, in some embodiments. In this example, such distances may be monitored by the data processing device system (e.g., 110, 310) by employing those same transducers themselves. In some embodiments, such distances may be monitored via other devices or transducers (e.g., the device location tracking systems of FIG. 2 or 3 in some embodiments). Such other transducers may be considered at least part of a third transducer set in some embodiments.

In this regard, the data set indicative of separation between the second transducer set and the tissue surface per block 804 may be derived in various manners, according to various embodiments. For example, in some embodiments, the input-output device system (e.g., 120, 320) may include a third transducer set, the third transducer set including at least a proximity sensor configured to determine a distance from the proximity sensor to the tissue surface, and the data set indicative of separation between the second transducer set of the transducer-based device (e.g., 200, 300, or 400) and the tissue surface of the bodily cavity is determined based at least on an analysis (e.g., performed by the data-processing device system (e.g., 110, 310)) of a signal set provided by the proximity sensor. In some embodiments, the second transducer set includes the third transducer set. According to various embodiments, the proximity sensor may take the form of various devices and may sense a distance between itself and the second transducer set, a distance between the second transducer set and the tissue surface, or a distance between the proximity sensor and the tissue surface based on various technologies including those described in this disclosure. For example, the proximity sensor may, in some embodiments, be an ultrasonic sensor. In some embodiments, the transducer-based device (e.g., 200, 300, or 400) includes the proximity sensor. In some embodiments, the proximity sensor may be employed to determine a distance between itself and various portions of the tissue surfaces of the bodily cavity and may provide that information as a signal set to the data processing device system. In this regard, in some embodiments, a magnetic field sensor 277 may be considered such a proximity sensor that detects its position in three-dimensional space, such detection of position may be provided as the signal set to the data processing device system (e.g., 110, 310) to indicate or be utilized by the data processing device system at least to determine distances between the magnetic field sensor 277 and other objects (e.g., transducers of the transducer-based device or a tissue wall of the bodily cavity) in such three-dimensional space, according to some embodiments. In this regard, with such a signal set from, e.g., a proximity sensor, and possibly with knowledge of the geometry of the transducer-based device (e.g., 200, 300, or 400), and possibly with knowledge of a computer-based model of the geometry of the bodily cavity, a distance or degree of proximity between the second transducer set (or the first transducer set in some embodiments) and the adjacent tissue surface of the bodily cavity may be determined based on an analysis of the signal set.

In some embodiments, the input-output device system (e.g., 120, 320) includes a device location tracking system (e.g., 260A, 260B as described as per FIG. 2 or 3 above). In some embodiments, the data processing device system (e.g., 110, 310) is configured at least by the program at least to cause, via the input-output device system (e.g., 120, 320), reception of a location signal set from the device location tracking system, the location signal set indicating a location of at least one transducer in the second transducer set. According to various embodiments, the data set per at least block 804 is derived at least in part from the location signal set. In some embodiments, the device location tracking system (e.g., 260A, 260B in some embodiments) is configured to provide the location signal set to (and consequently, received by) the controller 324 or its data processing device system (e.g., 110, 310). According to various embodiments, the location signal set may indicate a plurality of locations in a bodily cavity in response to movement of at least part of a transducer-based device (e.g., transducer-based device 200, 300, or 400 in some embodiments) in the bodily cavity. In some embodiments, the location signal set may be indicative of at least a particular location of the plurality of locations. In some embodiments, the device location tracking system (e.g., 260A) is configured to generate the location signal set at least in response to one or more electric fields producible by one or more devices of the device location tracking system. For example, with respect to FIG. 2, a location of at least one transducer in the second transducer set (e.g., transducer-based device 200, 300, or 400) may be determined in the presence of an electric field set (e.g., one or more electric fields generated by the electrodes 256a, 256b, 256c, 256d, 256e, 256f). According to various embodiments, the electrodes 256a, 256b, 256c, 256d, 256e, 256f are one or more devices of the device location tracking system that are external or are configured to operate outside a body that includes the bodily cavity. In some embodiments, the device location tracking system (e.g., 260B) is configured to generate the location signal set at least in response to one or more magnetic fields producible by one or more devices of the device location tracking system. For example, with respect to FIG. 3, a location of at least one transducer in the second transducer set (e.g., transducer-based device 200, 300, or 400) may be determined in the presence of a magnetic field set (e.g., one or more magnetic fields generated by magnetic field generation sources 257w, 257x, 257y). According to various embodiments, the magnetic field generation sources 257w, 257x, 257y are one or more devices of the device location tracking system that are external or are configured to operate outside a body that includes the bodily cavity. In various embodiments, the device location tracking system may be deemed to include the respective transducer(s) (e.g., transducers 220, 306, 406 (or, e.g., 277 in the case of some magnetic-field-based systems)) that detected the field strength(s), the field-generating devices (e.g., the external electrodes 256a, 256b, 256c, 256d, 256e, 256f in the case of electric field(s); and, e.g., magnetic field generation sources 257w, 257x, 257y in the case of magnetic field(s)), or both the respective transducers and the field-generating devices. In some embodiments, the controller 324 or its data processing device system 310 may be considered at least part of the device location tracking system.

In some embodiments, the location signal set provided by the device location tracking system (e.g., 260A or 260B in some embodiments) may indicate movement of at least a portion of a transducer-based device (e.g., transducer-based device 200, 300, or 400 in some embodiments) through or between a plurality of locations in a bodily cavity. In some embodiments, the location signal set may form part of a plurality of location signal sets, each location signal set may be indicative of a respective location of the plurality of locations. In some embodiments, each location signal set may be indicative of a respective location in a sequence of locations at which at least a portion of a catheter (e.g., at least one transducer of the second transducer set, in some embodiments) has been sequentially located in a bodily cavity, according to some embodiments. For example, with respect to at least FIG. 2 or FIG. 3, at least a portion of the catheter or transducer-based device (e.g., transducer-based device 200, 300, or 400) may be moved or progressed through a sequence of locations in a chamber of the heart or other bodily cavity of the patient 361 (or through a quality-control, training, or testing environment) in the presence of an electric field set (e.g., one or more electric fields generated by the external electrodes 256a, 256b, 256c, 256d, 256e, 256f) or a magnetic field set (e.g., one or more magnetic fields generated by magnetic field generation sources 257w, 257x, 257y). As the portion of the catheter is moved through the sequence of locations, at least some of the catheter's transducers (e.g., transducers 220, 306, 406 (or, e.g., 277 in the case of magnetic-field-based systems)) may be configured to generate each location signal set as detected strengths of the respective field(s), which the controller 324 or its data processing device system 310 may then be configured to utilize to generate a three-dimensional (“3D”) location of the at least the portion of the catheter (e.g., transducer-based device 200, 300, or 400) or its transducers (e.g., transducers 220, 306, 406 (and, e.g., 277 in the case of magnetic-field-based systems)) for the respective location in the sequence of locations, according to some embodiments.

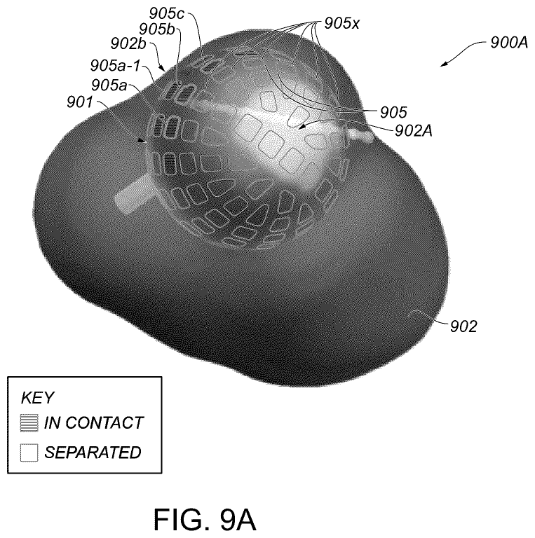

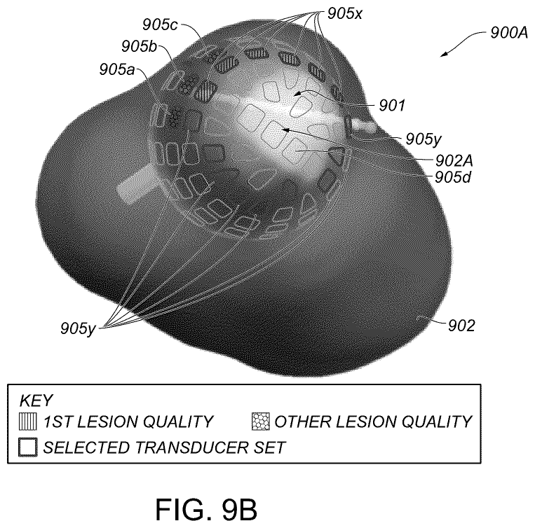

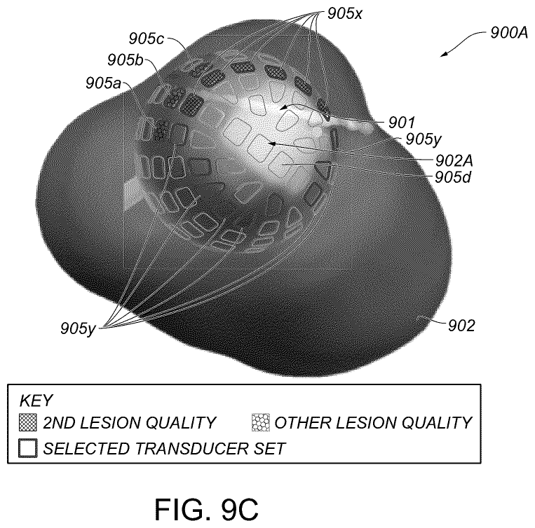

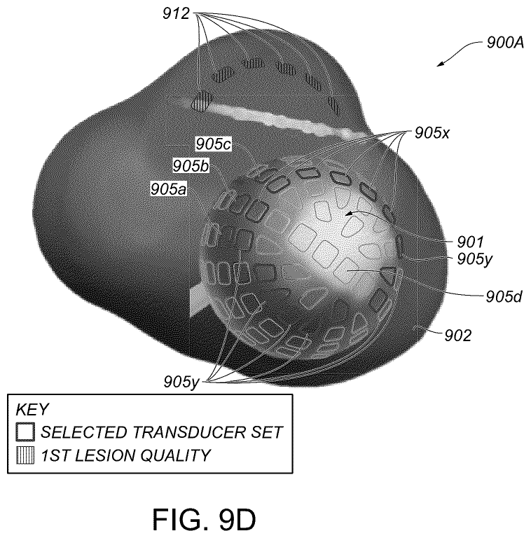

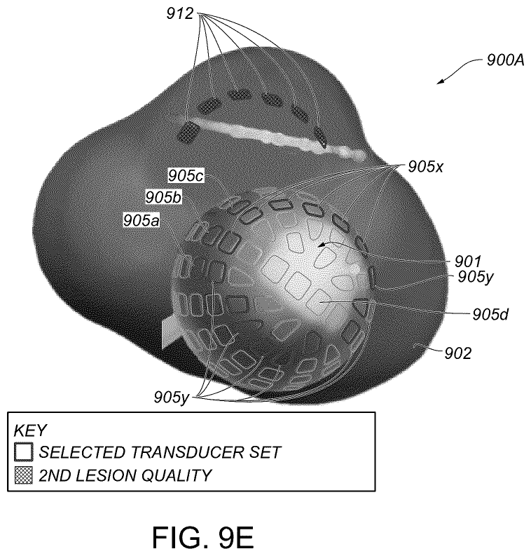

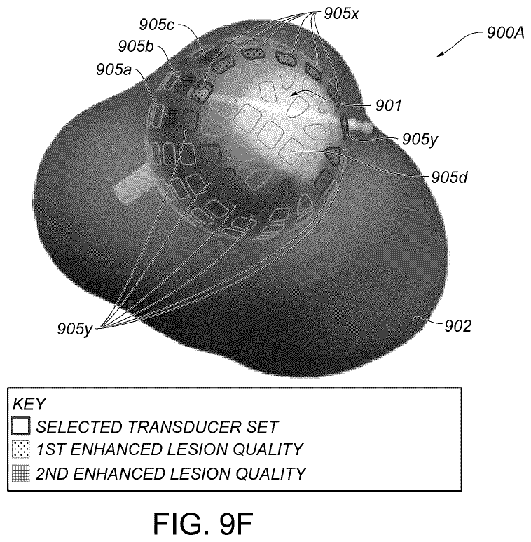

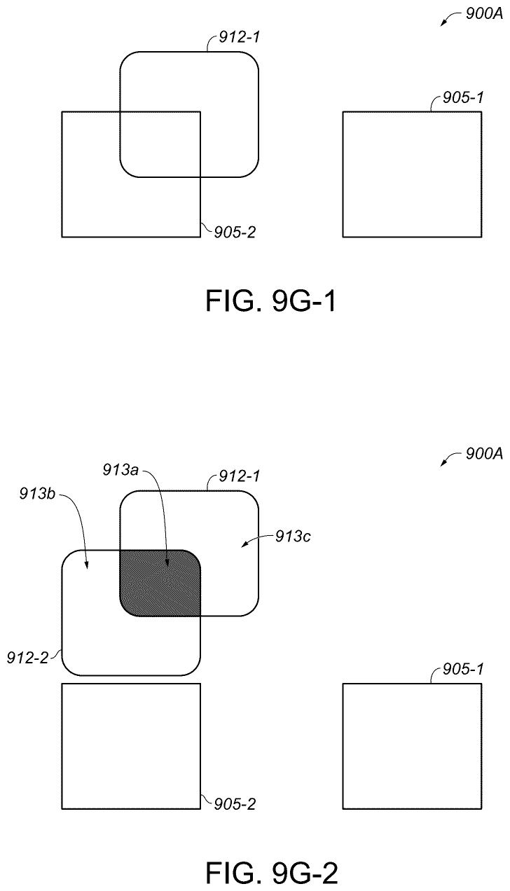

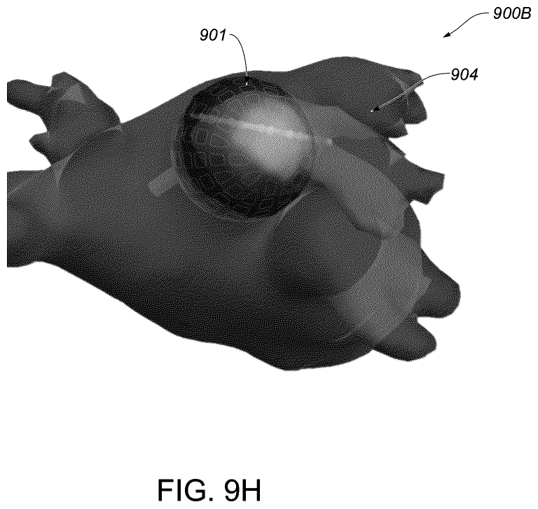

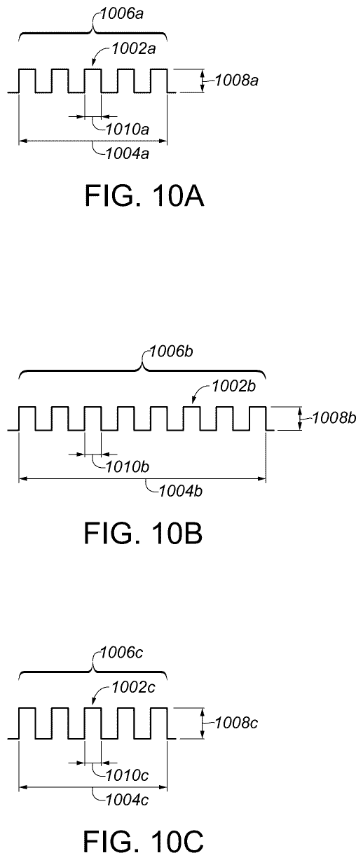

In some embodiments, a 3D graphical representation, envelope, or model of the bodily cavity may be generated from the location signal set when the sequence of locations are locations in which the at least the portion of the catheter or transducer-based device interacted (e.g., via contact) with a particular region of the tissue surface of the bodily cavity. From this interaction, the 3D graphical representation, envelope, or model may define a location of the particular region of the tissue surface of the bodily cavity. For example, according to some embodiments, FIG. 9A shows at least part of a graphical user interface including a display of a 3D graphical representation 900A that includes a transducer-based device representation 901 (e.g., a representation of at least part of a transducer-based device similar to that shown in FIGS. 4 and 6) in a representation of an envelope 902 generated at least in part from location information determined based at least on a location signal set provided by a device location tracking system (e.g., 260A). According to various embodiments, the locations of various regions of the tissue surface may be determined and combined as part of a process of generating a graphical or computer-based model (e.g., a three-dimensional model or envelope) representing the bodily cavity. In some embodiments, the envelope representing the bodily cavity takes the form of a representation of a pre-existing image or model, such as a CT scan, of the cavity. For example, according to some embodiments, FIG. 9H shows at least part of a graphical user interface including a display of a 3D graphical representation 900B that includes a transducer-based device representation 901 (e.g., a representation of at least part of a transducer-based device similar to that shown in FIGS. 4 and 6) in a representation of a pre-existing model of an envelope 904 that was generated from a CT scan. According to various embodiments, the pre-existing image may be registered in a graphical space that maps the location information.

In some embodiments, the data processing device system (e.g., 110, 310) may be configured at least by the program at least to cause display, via the input-output device system (e.g., 120, 320), of an envelope representing a bodily cavity and a representation of the transducer-based device (e.g., 200, 300, or 400) located in proximity to the envelope. According to some embodiments, the data processing device system (e.g., 110, 310) may be configured at least by the program at least to derive the data set (referred to, e.g., in at least block 804 in FIG. 8A) at least in part from an analysis of information corresponding to a distance between at least part of the representation of the transducer-based device (e.g., 200, 300, or 400) and a portion of the envelope adjacent the at least part of the transducer-based device. In some embodiments, the envelope is provided by a pre-existing image (e.g., a CT scan). In some embodiments, the envelope is generated with the assistance of a device location system (e.g., 260A, 260B) in a manner similar to, or the same as, that described above.

In some embodiments, the input-output device system (e.g., 120, 320) includes a device location tracking system (e.g., 260A, 260B), and the data processing device system (e.g., 110, 310) is configured at least by the program (e.g., per program instructions associated with some embodiments of at least block 804) at least to perform the analysis of the information corresponding to the distance between the at least part of the representation of the transducer-based device (e.g., 200, 300, or 400) and the portion of the envelope adjacent the at least part of the representation of the transducer-based device based at least on a location signal set provided by the device location tracking system (e.g., 260A, 260B). For example, with reference to FIG. 9A, the device location tracking system (e.g., 260A, 260B) may provide a location signal set to the data processing device system (e.g., 110, 310) that indicates a location (e.g., a coordinate or region in a three-dimensional space) of the second transducer set referred to in block 804. In FIG. 9A, the second transducer set may be represented at least in part by transducer 905a-1. In addition, the data processing device system may have stored in memory (e.g., processor-accessible memory device system 130, 330) a location in three-dimensional space of the envelope 902. With these two pieces of information, i.e., the location of the second transducer set (from the location signal set) and the location of the envelope 902, the data processing device system can determine a distance between the second transducer set (e.g., at least transducer 905a-1) and a closest surface (e.g., 902b in FIG. 9A) of the envelope 902, which may represent a tissue surface of the bodily organ. Accordingly, in some embodiments, the data processing device system (e.g., 110, 310) is configured at least by the program (e.g., per program instructions associated with some embodiments of at least block 804) at least to perform the analysis of the information corresponding to the distance between the at least part of the representation of the transducer-based device (e.g., 200, 300, or 400) and the portion of the envelope adjacent the at least part of the representation of the transducer-based device based at least on a location signal set provided by the device location tracking system (e.g., 260A, 260B).

In some embodiments, the data processing device system (e.g., 110, 310) may be configured at least by the program at least to determine a location of the at least part of the representation of the transducer-based device (e.g., 200, 300, or 400) (e.g., transducer 905a-1 in the example of FIG. 9A) based at least on a first location signal set provided by the device location tracking system, and to determine a location of the portion of the envelope (e.g., portion 902b in the example of FIG. 9A) adjacent the at least part of the representation of the transducer-based device based at least on a second location signal set provided by the device location tracking system (e.g., 260A, 260B). In this regard, the location of the portion of the envelope adjacent the at least part of the representation of the transducer-based device may be determined from a second location signal set provided by the device location tracking system (e.g., 260A, 260B) that was employed to define the envelope in graphical space (for example, as described above in this disclosure) according to some embodiments. For instance, boundaries of the envelope (e.g., envelope 902 in FIG. 9A) may be derived from the second location signal set provided by a device location tracking system (e.g., 260A, 260B) during states of contact between at least part of the transducer-based device and the tissue wall of the bodily cavity. An example of the first location signal set may be the location signal(s) provided by the device location tracking system (e.g., 260A, 260B) to provide the data processing device system the location of the at least part of the representation of the transducer-based device (e.g., transducer 905a-1 in the example of FIG. 9A), in some embodiments. In some embodiments, the location of the portion of the envelope in three-dimensional space may correspond to the second location signal set provided by the device location tracking system (e.g., 260A, 260B). In some embodiments, the location of the portion of the envelope (e.g., envelope portion 902b in the example of FIG. 9A) in three-dimensional space corresponds to an interpolated location on the envelope (e.g., envelope 902) that was derived from the second location signal set and one or more location signal sets provided by the device location tracking system (e.g., 260A, 260B) for the derivation of the envelope. In some embodiments, in which the envelope corresponds to an image that was not derived from location signal sets provided by the device location tracking system (e.g., 260A, 260B) (for example, a CT scan), the image may be registered in graphical space with respect to the graphical space coordinate frame that corresponds to the physical coordinate space employed by the device location tracking system (e.g., 260A, 260B).

As discussed above, in some embodiments, the data processing device system (e.g., 110, 310) may be configured at least by the program at least to derive the data set (referred to, e.g., in at least block 804 in FIG. 8A) at least in part from an analysis of information corresponding to a distance between at least part of a representation of the transducer-based device (e.g., 200, 300, or 400) and a portion of the envelope adjacent the at least part of the transducer-based device. According to some embodiments, the at least part of the representation of the transducer-based device (e.g., 200, 300, or 400) is a representation of the second transducer set referred to at least in block 804. In some embodiments, the at least part of the representation of the transducer-based device (e.g., 200, 300, or 400) corresponds to the second transducer set. In some embodiments, the second transducer set is at least one transducer (e.g., transducer 206, 306, 406, or 277) and the at least part of the representation of the transducer-based device (e.g., 200, 300, or 400) corresponds to the at least one transducer. In some embodiments, the second transducer set includes multiple transducers (e.g., transducer 206, 306, 406, or 277), and the at least part of the representation of the transducer-based device (e.g., 200, 300, or 400) includes a respective portion corresponding to each of the multiple transducers. In some embodiments, the second transducer set is a spatial distribution of multiple transducers (e.g., transducer 206, 306, 406, or 277), and the at least part of the representation of the transducer-based device (e.g., 200, 300, or 400) corresponds to one or more locations in the spatial distribution. For example, the at least part of the representation of the transducer-based device (e.g., 200, 300, or 400) may, as discussed above, correspond to a geographic center or a centroid (e.g., an average physical or virtual location) associated with the spatial distribution that may be determined based on locations of each of the multiple transducers. In some embodiments, the geographic center or the centroid corresponds to a location in the spatial distribution spaced from the tissue surface according to some embodiments. In some embodiments, the second transducer set is at least a part of the first transducer set (referred to at least in block 802). In some embodiments, the at least part of the representation of the transducer-based device (e.g., 200, 300, or 400) corresponds to the second transducer set (referred to at least in block 804). In this regard, in some embodiments, the first transducer set and the second transducer set may be the same, such that, for example, the same transducer set that delivers the first high voltage pulse set to cause pulsed field ablation per block 802, in some embodiments, is the same transducer set whose separation from the tissue surface in the bodily cavity is monitored per block 804, in some embodiments. However, in other embodiments, the first transducer set and the second transducer set are partially or entirely different, such that at least one transducer that delivers the first high voltage pulse set to cause pulsed field ablation per block 802, in some embodiments, does not have its separation from the tissue surface in the bodily cavity monitored per block 804, in some embodiments, and vice versa, in some embodiments.

In FIG. 8A, according to some embodiments, block 806 represents a configuration of the data processing device system (e.g., data processing device systems 110 or 310) (e.g., according to a program) to cause, based at least on an analysis of a particular degree of separation between the second transducer set and the tissue surface indicated by the data set, determination of a quality of lesion producible in the tissue (which may include the tissue surface) by the first high voltage pulse set. For example, according to some embodiments, each of block 806-1 and block 806-2 represents a possible implementation of at least part of block 806 in a respective state, according to some embodiments. Block 806-1 represents a configuration of the data processing device system (e.g., data processing device systems 110 or 310) (e.g., according to a program) to cause, based at least on an analysis of the data set (referred to at least in block 804), (a) determination, at least in response to a first state in which the analysis of the data set is indicative of a first degree of separation between the second transducer set and the tissue surface, of a first quality of lesion producible in the tissue by the first high voltage pulse set. Block 806-2 represents a configuration of the data processing device system (e.g., data processing device systems 110 or 310) (e.g., according to a program) to cause, based at least on an analysis of the data set, (b) determination, at least in response to a second state in which the analysis of the data set is indicative of a second degree of separation between the second transducer set and the tissue surface, of a second quality of lesion producible in the tissue by the first high voltage pulse set.

In some embodiments, block 806 may be considered to represent a configuration of the data processing device system (e.g., data processing device system 110 or 310) (e.g., according to a program) to vary a determination of a quality of lesion producible in the tissue by the first high voltage pulse set based at least on different degrees of separation between the second transducer set and the tissue surface as indicated by the data set monitored per block 804. In some embodiments, these varying determinations of the quality of lesion may manifest as the different first and second qualities of the lesion referred to in blocks 806-1, 806-2. For instance, if the first degree of separation referred to in block 806-1 is a lower degree of separation than the second degree of separation referred to in block 806-2, then, all else being equal, the data processing device system may be configured to determine that the first quality of lesion in the first state of block 806-1 is better (e.g., resulting in greater tissue damage) than the second quality of lesion in the second state of block 806-2, due to the lesser separation between the second transducer set and the tissue surface in the first state. Other factors besides mere degree of separation may be considered, however, in determining a quality of a lesion, as discussed in more detail below, according to some embodiments.

In FIG. 8A, according to some embodiments, block 808 represents a configuration of the data processing device system (e.g., data processing device systems 110 or 310) (e.g., according to a program) to cause, via the input-output device system (e.g., input-output device systems 120, 320), at least in response to the determination of a quality of lesion producible in the tissue by the first high voltage pulse set per at least block 808, display of a graphical element set indicating the determined quality of lesion. For example, according to some embodiments, each of block 808-1 and block 808-2 represents a possible implementation of at least part of block 808 in a respective state, according to some embodiments. Block 808-1 represents a configuration of the data processing device system (e.g., data processing device systems 110 or 310) (e.g., according to a program) to cause, via the input-output device system (e.g., input-output device systems 120, 320), (i) at least in response to the determination of the first quality of lesion producible in the tissue by the first high voltage pulse set per block 806-1, display of a first graphical element set indicating the determined first quality of lesion, according to some embodiments. Block 808-2 represents a configuration of the data processing device system (e.g., data processing device systems 110 or 310) (e.g., according to a program) to cause, via the input-output device system (e.g., input-output device systems 120, 320), (ii) at least in response to the determination of the second quality of the lesion producible in the tissue by the first high voltage pulse set per block 806-2, display of a second graphical element set indicating the determined second quality of the lesion, according to some embodiments. It is noted that there is an interaction between block 806-1 and 808-1, and an interaction between block 806-2 and 808-2. These interactions are not visually indicated in FIG. 8A, but exist nonetheless according to some embodiments.

Each of the first graphical element set per block 808-1 and the second graphical element set per block 808-2 may take various forms, according to some embodiments. For example, in some embodiments, each graphical element or graphical element subset in (1) the first graphical element set, (2) the second graphical elements set, or each of (1) and (2) corresponds to a respective transducer of the transducer-based device (e.g., 200, 300, 400). In some embodiments, each graphical element or graphical element subset in (1) the first graphical element set, (2) the second graphical elements set, or each of (1) and (2) corresponds to a respective transducer in the first transducer set. For example, in some embodiments, (1) the first graphical element set, (2) the second graphical elements set, or each of (1) and (2) may include a text-based indicator set indicating the respective quality of lesion. In some embodiments, (1) the first graphical element set, (2) the second graphical elements set, or each of (1) and (2) may include an icon-based or symbol-based indicator set indicating the respective quality of lesion. Without limitation, (1) the first graphical element set, (2) the second graphical elements set, or each of (1) and (2) may include any graphical form that can identify to a user the respective quality of lesion. In some embodiments, the second graphical element set is distinct from the first graphical element set. For example, in some embodiments, the second graphical element set includes at least one graphical element that is different or other than each graphical element in the first graphical element set. In some embodiments, each graphical element in the second graphical element set is different or other than each graphical element in the first graphical element set. In some embodiments, a visual characteristic set of the second graphical element set is different than a visual characteristic set of the first graphical element set. In some embodiments, (1) the first graphical element set, (2) the second graphical element set, or each of (1) and (2) corresponds to the first transducer set. In some embodiments, the first transducer set is the second transducer set, and (1) the first graphical element set, (2) the second graphical element set, or each of (1) and (2) corresponds to the first transducer set. In some embodiments, each respective transducer in the first transducer set corresponds to a respective one or more graphical elements in (1) the first graphical element set, (2) the second graphical element set, or each of (1) and (2). In this regard, each graphical element in (1) the first graphical element set, (2) the second graphical element set, or each of (1) and (2) may be representative of at least one transducer in the first transducer set according to some embodiments.

In some embodiments, each graphical element or graphical element subset in (1) the first graphical element set, (2) the second graphical element set, or each of (1) and (2) corresponds to a location where delivery of the first high voltage pulse is to occur, is occurring, or has occurred. In some embodiments, (1) the first graphical element set, (2) the second graphical element set, or each of (1) and (2) is determined by the data processing device system (e.g., 110, 310) based on the locations of at least some of the transducers in the first transducer set during delivery of the first high voltage pulse set. In some embodiments, each graphical element or graphical element subset in (1) the first graphical element set, (2) the second graphical element set, or each of (1) and (2) corresponds to or indicates a location of a respective transducer or respective group of transducers in the first transducer set during delivery of the first high voltage pulse set. For example, in some embodiments, each graphical element in (1) the first graphical element set, (2) the second graphical element set, or each of (1) and (2) includes displayed information that defines coordinates of a respective transducer or respective group of transducers in the first transducer set during the delivery of the first high voltage pulse set. In some embodiments, each graphical element or graphical element subset in (1) the first graphical element set, (2) the second graphical element set, or each of (1) and (2) is mapped to a particular respective location in a displayed graphical representation indicating a location where the first high voltage pulse set is delivered. In some embodiments, each graphical element or graphical element subset in (1) the first graphical element set, (2) the second graphical element set, or each of (1) and (2) is mapped to a particular respective location in a displayed graphical representation indicating a location of a respective transducer in the first transducer set during the delivery of the first high voltage pulse set. For example, in some embodiments, the data processing device system (e.g., 110, 310) is configured at least by the program at least to cause display, via the input-output device system (e.g., 120, 320) of a map of the tissue surface, and cause display, via the input-output device system, of (1) the first graphical element set, (2) the second graphical element set, or each of (1) and (2) at one or more locations on the map of the tissue surface corresponding to one or more locations where the first high voltage pulse set is delivered, e.g., one or more locations on the tissue surface at which at least part of the lesion is formed or formable by delivery of the first high voltage pulse set, according to some embodiments.

In FIG. 9A, the graphical representation 900A includes an envelope 902 that shows a map of a tissue surface (e.g., a 3D map), according to some embodiments. The transducer-based device representation 901 shown positioned within the envelope 902 corresponds to a transducer-based device (e.g., 200, 300, or 400), according to various embodiments. According to some embodiments, the transducer-based device representation 901 shown positioned within the envelope 902 corresponds to a first particular positioning of the transducer-based device (e.g., 200, 300, or 400) within a bodily cavity represented by envelope 902. According to some embodiments, the transducer-based device representation 901 includes a plurality of transducer graphical elements 905 (two called out in FIG. 9A), each transducer graphical element corresponding to a respective transducer of a plurality of transducers (e.g., transducers 206, 306, or 406, in some embodiments) that form part of the transducer-based device (e.g., 200, 300, or 400). In various embodiments associated with FIG. 9A, the transducer-based device representation 901 is positioned at the first particular positioning adjacent a region 902A of envelope 902 that corresponds to a port in a bodily cavity that the envelope 902 corresponds to or represents. In some embodiments, the bodily cavity is a cardiac cavity, and the port corresponding to region 902A represents a port of a pulmonary vein. In FIG. 9A, particular ones of the graphical elements 905 that correspond to the particular transducers of the transducer-based device (e.g., 200, 300, or 400) that are in contact with tissue are indicated with a particular visual characteristic set exemplified in the KEY shown in FIG. 9A. In FIG. 9A, particular ones of the graphical elements 905 that correspond to the particular transducers of the transducer-based device (e.g., 200, 300, or 400) that are separated from tissue are indicated with a particular visual characteristic set exemplified in the KEY shown in FIG. 9A. Determination of which particular transducers of the transducer-based device (e.g., 200, 300, or 400) that are in contact with tissue and which particular transducers of the transducer-based device (e.g., 200, 300, or 400) that are separated from the tissue may occur in various manners including the proximity detection techniques described above in this disclosure.

FIG. 9B shows the graphical representation 900A of FIG. 9A with the transducer-based device representation 901 again shown positioned in the envelope 902 in a manner corresponding to the first particular positioning of the transducer-based device (e.g., 200, 300, or 400) within the bodily cavity represented by envelope 902. FIG. 9B shows the graphical representation 900A of FIG. 9A in a state in which a set of transducers (e.g., transducers 206, 306, or 406) of the transducer-based device (e.g., 200, 300, or 400) has been selected, their corresponding transducer graphical elements shown with a bolded outline to indicate their selection, according to some embodiments. According to some embodiments, the selected set of transducers (e.g., transducer 206, 306, or 406) is selected to deliver pulsed field ablation energy (e.g., via the delivery of a high voltage pulse set). According to some embodiments, some, but not all, of the plurality of transducers have been selected to deliver pulsed field ablation energy. In some embodiments, the set of transducers are selected at least in response to reception by the data processing device system (e.g., 110, 310) of user input provided via the input-output device system (e.g., 120, 320). In some embodiments, the set of transducers are selected at least in response to a machine-based selection. A machine-based selection may include or be, for example, a selection of one or more transducers executed by the data processing device system (e.g., 110, 310) based on an analysis of information, such as information from one or more transducers, according to some embodiments. For instance, the data processing device system may be configured to analyze bodily cavity map information to determine the location of a port in the bodily cavity. Also, based on tissue proximity and transducer-location information from a device location tracking system (e.g., per FIG. 2 or FIG. 3), the data processing device system may be configured to automatically select the transducers surrounding the port. It is noted that in some embodiments, all of the transducers (e.g., transducer 206, 306, or 406) of a transducer-based device (e.g., 200, 300, or 400) may be operated to deliver pulsed field ablation energy. It is noted that such operation of all the transducers is considered, according to various embodiments, to inherently include a selection of all of the transducers.

It is noted that, if a transducer set (e.g., the first transducer set referred to in at least block 802 in FIG. 8A) is selected to deliver thermal energy (e.g., RF energy), typically the one or more transducers in such selected transducer set should be in contact with tissue to increase the quality of the lesion(s) that is to be formed and to reduce the risk of deleterious effects, such as thermal coagulum. Pulsed field ablation (“PFA”), on the other hand, does not typically cause thermal coagulum and, as such, transducers that are not in contact with tissue, but rather are separated from the tissue can form at least part of the selection of the transducer set that is to perform PFA (e.g., the first transducer set referred to in at least block 802 in FIG. 8A). The selection of particular transducers that are separated from tissue may be motivated for different reasons. For example, although transducers that are in contact with tissue may be included in the selected transducer set indicated by FIG. 9B to provide a good chance of creating efficacious lesions in response to the delivery of a high voltage pulse set, inclusion of additional neighboring transducers can further increase the chances that the quality of the lesions will be further improved even if the additional neighboring transducers are not in tissue contact. In this regard, according to various embodiments, the selected transducer set in the example of FIG. 9B may include transducers that are separated from the tissue surface, according to some embodiments.

FIG. 9B shows three transducer graphical elements 905a, 905b, and 905c whose corresponding transducers form part of the selected transducer set and are in contact with tissue. Such contact is illustrated in FIG. 9A (not FIG. 9B and also not in FIGS. 9C-9F) by the internal horizontal parallel line pattern used for graphical elements 905a, 905b, and 905c. The contact pattern is not shown in FIG. 9B for purposes of clarity so that there are not too many different internal graphical element patterns shown at once. However, such contact (and separation discussed below) may nonetheless be visually presented to a user via a graphical user interface showing the state of FIG. 9B, in some embodiments. FIG. 9B also shows that each of the transducer graphical elements indicated as 905x (six called out in FIG. 9B) corresponds to a respective transducer in the selected transducer set, which is separated from (not in contact with) the tissue surface. Such separation is illustrated in FIG. 9A (not FIG. 9B and not FIGS. 9C-9F) by the non-usage of an internal pattern used for graphical elements 905x. The lack of usage of an internal pattern used for any graphical element 905 in FIGS. 9B-9F should not be construed to indicate no tissue contact, as examples of the contact/separation states are intended to be illustrated with FIG. 9A instead. While selected graphical elements 905a, 905b, 905c, and 905x have been called out in this example with respect to FIG. 9B, other selected graphical elements are shown in FIG. 9B, including graphical elements 905y. However, many of the examples described herein will focus on graphical elements 905a, 905b, 905c, and 905x, and not the other selected graphical elements merely for ease of discussion. Further, while FIG. 9A shows graphical elements that are separated from tissue with only a single type of graphical element or visual characteristic (i.e., the non-usage of an internal pattern for the respective graphical elements 905), different graphical elements or visual characteristics may be utilized in some embodiments to illustrate particular degrees of separation exhibited by the corresponding transducers. The same applies for degrees of contact in some embodiments.

Determination of which particular ones of the transducers of the transducer-based device (e.g., 200, 300, or 400) that are in contact with tissue and which particular ones of the transducers of the transducer-based device (e.g., 200, 300, or 400) are separated from the tissue may occur in various manners including the proximity detection techniques described above in this disclosure. It is noted that, in FIG. 9B, although not all the transducer graphical elements 905 that correspond to transducers that are separated from tissue have been selected as part of the selected transducer set, the inclusion of at least some of these other non-tissue contacting transducers as part of the selected transducer set may occur, according to some embodiments. For example, a transducer located over a port (e.g., a transducer corresponding to transducer graphical element 905d) may form part of the selected transducer set. In some embodiments, as discussed above, the topography of the bodily cavity and the geometry of the transducer-based device may be such that there is little choice other than including a non-tissue contacting transducer as at least part of the selected set of transducers. In this regard, the present inventors recognized that it may be important to be able to provide a determination of an actual or expected lesion quality and an indication to a user of the determined actual or expected lesion quality in instances in which transducers are separated from tissue, as provided by some embodiments of the present invention.

It should be noted that, although some embodiments described herein, in which a transducer selected and operated to perform tissue ablation is separated from the tissue surface, are described in the context of PFA, other embodiments may include such a selection and operation of a transducer separated from the tissue surface for other forms of ablation, including, in some instances, thermal ablation. While particular care may need to be taken in some contexts in which thermal ablation is performed with a transducer separated from tissue to avoid thermal coagulum, such as by limiting thermal energy delivered and increasing ablation time at the lower thermal energy level, such an ablation may nonetheless occur (for example, in irrigated thermal ablation systems). In this regard, it is still beneficial to provide a determination of an actual or expected lesion quality and an indication to a user of the determined actual or expected lesion quality in instances in which transducers are separated from tissue and are performing another type of ablation besides PFA.

According to some embodiments associated with FIG. 9B, the first transducer set caused or to be caused to be activated or operated by the data processing device system (e.g., 110, 310) to deliver the first high voltage pulse set per block 802 of FIG. 8A may include or be, for purposes of the example of FIG. 9B, the transducers associated with graphical elements 905x. Other transducers of the selected transducers shown in FIG. 9B may also be caused to be activated or operated by the data processing device system (e.g., 110, 310) to deliver the first high voltage pulse set or another high voltage pulse set, in some embodiments. According to some embodiments associated with FIG. 9B and block 806-1 of FIG. 8A, the first particular positioning of the transducer-based device (e.g., 200, 300, or 400) within the bodily cavity represented by envelope 902 may correspond to the first state in which the analysis of the data set is indicative of the first degree of separation between the second transducer set (e.g., whose degree of separation is monitored per at least block 804) and the tissue surface. In the example of FIG. 9B, the second transducer set and the first transducer set (that delivers or is selected to deliver the first high voltage pulse set per at least block 802) are the same, although that need not be the case as described herein. According to various embodiments, the first degree of separation corresponds to separation between the second transducer set and the tissue surface. In some embodiments, the second transducer set (which is also the first transducer set in this example of FIG. 9B, but need not be in some embodiments) corresponds to transducer graphical elements 905x, which, per FIG. 9A, are indicated as having associated transducers that are separated from tissue. According to various embodiments, analysis of the data set (monitored per at least block 804) in accordance with block 806-1 indicates that the first quality of lesion is producible when the transducers corresponding to the transducer graphical elements 905x are activated as the first transducer set to deliver the first high voltage pulse set. Example analyses of determining such a quality of lesion are discussed in more detail below.

Referring to block 808-1 in FIG. 8A, the first graphical element set indicating the first quality of lesion producible in the tissue by the first high voltage pulse set is displayed according to some embodiments. For example, in FIG. 9B, each of the transducer graphical elements 905x may be operated as the first transducer set to deliver the first high voltage pulse set to cause pulsed field ablation of tissue. As indicated above in this disclosure, in some embodiments, analysis of the data set in accordance with block 806-1 indicates that the first quality of lesion is producible when the transducers corresponding to the transducer graphical elements 905x are activated as the first transducer set to deliver the first high voltage pulse set. In this regard, according to some embodiments, the first quality of lesion is indicated in FIG. 9B by having each particular transducer graphical element 905x appear as having the internal vertical parallel line pattern identified by the “1ST LESION QUALITY” entry in the KEY in FIG. 9B.

In some embodiments, the manner in which the graphical elements are graphically produced for selected transducers may indicate the determined (e.g., per at least block 808) expected or actual lesion quality based at least on the selected transducers' degree of separation from tissue. While FIG. 9B illustrates a simple example of only one pattern (represented in the KEY for “1ST LESION QUALITY”) to illustrate all transducers associated with transducer graphical elements 905x as having a same degree of separation and resulting same lesion quality, other embodiments may vary the graphical elements or their visual characteristics in a more granular manner, where different degrees of separation cause display of different graphical elements or visual characteristics thereof. For instance, transducers that are separated from the tissue surface but are within 1.5 millimeters from the tissue surface may have corresponding graphical elements displayed with a symbol that gives the user a positive notion, such as a green circle; transducers that are separated from the tissue surface but are between 1.5 and 3 millimeters from the tissue surface may have corresponding graphical elements displayed with a symbol that gives the user a cautionary notion, such as a yellow triangle; and transducers that are separated from the tissue surface but are greater than 3 millimeters from the tissue surface may have corresponding graphical elements displayed with a symbol that gives the user a prohibited notion, such as a red octagon, like a stop sign, in some embodiments. Of course, other approaches and separation distance ranges may be utilized in other embodiments.

In FIG. 9C, the graphical representation 900A also includes envelope 902 that shows a map of a tissue surface (e.g., a 3D map) and the transducer-based device representation 901 corresponding to the transducer-based device (e.g., 200, 300, or 400) according to various embodiments. According to some embodiments, the transducer-based device representation 901 shown positioned within the envelope 902 corresponds to a second particular positioning of the transducer-based device (e.g., 200, 300, or 400) within a bodily cavity represented by envelope 902. In this regard, FIG. 9C may be considered to correspond to FIG. 9B, e.g., where the transducers associated with graphical elements 905x are considered the first transducer set referred to in block 802 in FIG. 8A, but in FIG. 9C, the transducer-based device representation 901 is shown positioned within the envelope 902 in a second particular positioning different than the first particular positioning shown in FIG. 9B. In this regard, the transducers associated with graphical elements 905x in the state of FIG. 9C have a different (e.g., a “second”) degree of separation from the tissue surface as compared to the (e.g., “first”) degree of separation from the tissue surface exhibited by those transducers in the state of FIG. 9B. Accordingly, FIG. 9C may be considered to be associated with block 806-2 in FIG. 8A, while FIG. 9B may be considered to be associated with block 806-1 in FIG. 8A, in some embodiments.

In this regard, according to some embodiments associated with FIG. 9C and block 806-2 of FIG. 8A, the second particular positioning of the transducer-based device (e.g., 200, 300, or 400) within the bodily cavity represented by envelope 902 corresponds to the second state in which the analysis of the data set is indicative of the second degree of separation between the second transducer set (e.g., whose degree of separation is monitored per at least at block 804) and the tissue surface. In the example of FIG. 9C, like FIG. 9B, the second transducer set and the first transducer set (that delivers or is selected to deliver the first high voltage pulse set per at least block 802) are the same, although that need not be the case as described herein. According to various embodiments, the second degree of separation corresponds to separation between the second transducer set and the tissue surface. In some embodiments, the second degree of separation corresponds to separation between each transducer corresponding respectively to a transducer graphical element 905x. According to various embodiments, analysis of the data set in accordance with block 806-2 indicates that the second quality of lesion is producible when the transducers corresponding to the transducer graphical elements 905x are activated as the first transducer set to deliver the first high voltage pulse set. Example analyses of determining such a quality of lesion are discussed in more detail below.

Referring back to block 808-2 in FIG. 8A, the second graphical element set indicating the second quality of lesion producible in the tissue by the first high voltage pulse set is displayed, according to some embodiments. In this regard, because the transducers associated with transducer graphical elements 905x in the second particular positioning of the transducer-based device (e.g., 200, 300, or 400) shown in FIG. 9C are relatively further away (appears to be a small amount to a layperson when comparing FIGS. 9B and 9C) from the tissue surface as compared to FIG. 9B, a lower quality lesion is determined in the second state of FIG. 9C per at least block 806-2 as the second quality of lesion as compared to the first state of FIG. 9B. To illustrate this second lesion quality in the second state of FIG. 9C as compared to the first state of FIG. 9B, transducer graphical elements 905x are displayed as including the internal intersecting line pattern (like a small diamond pattern) identified by the KEY in FIG. 9C as “2ND LESION QUALITY”.

While some of the above examples with respect to FIG. 9B and FIG. 9C are in the context of producing different graphical elements to illustrate different lesion qualities based on different degrees of separation, it is to be understood that what constitutes different graphical elements should be broadly interpreted to include changes in visual characteristics of what might be considered a same graphical element or a same type of graphical element. For instance, FIG. 9B illustrates transducer graphical elements that are associated with a first degree of separation as transducer graphical elements 905x that have an internal vertical parallel line pattern (indicating a first lesion quality), while FIG. 9C illustrates transducer graphical elements that are associated with a second degree of separation as transducer graphical elements 905x including an internal intersecting line diamond pattern (indicating a second lesion quality associated with the respective transducers' greater separation from tissue compared to the state of FIG. 9B). In this regard, it may be considered that the transducer graphical elements 905x in FIG. 9B and the transducer graphical elements 905x in FIG. 9C are different graphical elements, in some embodiments. Alternatively, or in addition, and also acceptably, it may be considered that the transducer graphical elements 905x in FIG. 9B and the transducer graphical elements 905x in FIG. 9C are the same graphical elements but with different visual characteristics, in some embodiments.

In this regard, in some embodiments, each of the first graphical element set (e.g., per at least block 808-1) and the second graphical element set (per at least block 808-2) includes at least one particular graphical element. In some of these embodiments, the data processing device system (e.g., 110, 310) may be configured at least by the program (e.g., per program instructions associated with at least block 808-1) at least to cause, via the input-output device system (e.g., 130, 330) and at least in response to the determination (per at least block 806-1) of the first quality of the lesion producible in the tissue by the first high voltage pulse set (referred to in at least block 802), display of the at least one particular graphical element with a first visual characteristic set. For example, in some embodiments, the transducer graphical elements 905x may be considered an example of the at least one particular graphical element, and the manner of displaying the transducer graphical elements 905x in FIG. 9B with the internal vertical parallel line pattern may be considered a display of such transducer graphical elements 905x with a first visual characteristic set. In some embodiments, the data processing device system (e.g., 110, 310) may be configured at least by the program (e.g., per program instructions associated with at least block 808-1) at least to cause, via the input-output device system (e.g., 130, 330) and at least in response to the determination (per at least block 806-2) of the second quality of the lesion producible in the tissue by the first high voltage pulse set, display of the at least one particular graphical element with a second visual characteristic set. For example, in some embodiments, the manner of displaying the transducer graphical elements 905x in FIG. 9C may be considered a display of such transducer graphical elements 905x with a second visual characteristic set that includes the internal intersecting line diamond pattern not present in the first visual characteristic utilized in the state of FIG. 9B.

Without limitation, the graphical representation in FIGS. 9, such as the graphical representation 900A in FIGS. 9B and 9C, may also include other graphical elements or the same graphical elements having other visual characteristic sets. For example, the other graphical elements or visual characteristic sets may, in some embodiments, include visual representations of a delivery or a status of delivery of pulsed field ablative energy or other ablative energy, according to some embodiments. In this regard, it is noted that the first graphical element set (per at least block 808-1), the second graphical element set (per at least block 808-2), or each of the first graphical element set and the second graphical element set is not limited to transducer graphical elements, and other forms of graphical elements may be employed in other embodiments. For example, in some embodiments, each graphical element in (1) the first graphical element set, (2) the second graphical element set, or each of (1) and (2) may be a graphical element representing at least part of a lesion formed in the tissue in response to the delivery of the first high voltage pulse set. In some embodiments, each graphical element in (1) the first graphical element set, (2) the second graphical element set, or each of (1) and (2) is mapped to a particular respective location in a displayed graphical representation indicating a location of a portion of lesion formed in tissue in response to the delivery of the first high voltage pulse set. In some embodiments, each graphical element in (1) the first graphical element set, (2) the second graphical element set, or each of (1) and (2) is mapped to a particular respective location in a displayed graphical representation indicating where pulsed field ablation (e.g., a part of the delivery of the first high voltage pulse set) or other ablative energy is delivered.

FIG. 9D shows the graphical representation 900A of FIG. 9B, but with the transducer-based device representation 901 repositioned relative to its location depicted in FIG. 9B. In this regard, the repositioned transducer-based device representation 901 indicates a repositioning of the transducer-based device (e.g., 200, 300, 400) from its first particular positioning as indicated in FIG. 9B. According to various embodiments, the transducer-based device (e.g., 200, 300, or 400) is repositioned from its first particular positioning after the activation or operation of the first transducer set (e.g., the transducers associated with graphical elements 905x, in this example, in some embodiments) to deliver the first high voltage pulse set to cause pulsed field ablation of tissue, e.g., per block 802 in FIG. 8A. In FIG. 9D, graphical elements 912 each correspond to a part of a lesion formed in response to the delivery of the first high voltage pulse set, each graphical element 912 having a first visual characteristic set made in response to the determination of the first quality of lesion as per block 806-1, as exemplified by the KEY in FIG. 9D, where the lesion markers 912 have a parallel-vertical-line internal pattern representing the first quality of lesion. According to some embodiments, the graphical elements 912 each correspond to a respective part of a lesion formed in response to the delivery of the first high voltage pulse set, and each graphical element 912 having a first visual characteristic set indicating that respective part of the lesion was determined to have the first quality of lesion. In some embodiments, each graphical element 912 is located in the graphical representation proximate a location of a corresponding one of the transducer graphical elements 905x during the delivery of the first high voltage pulse set. While the graphical elements 912 in FIG. 9D (and in FIG. 9E, discussed in more detail below) are separated from each other indicating discrete locations of lesion formation, other approaches may be taken in other embodiments. For example, since the lesion, in some embodiments, will likely be a continuous lesion, the graphical elements 912 may be graphically displayed as a continuous or overlapping graphical element set to illustrate the continuous nature of the lesion formed or to be formed.

In a similar manner, FIG. 9E shows the graphical representation 900A of FIG. 9C, but with the transducer-based device representation 901 repositioned relative to its location depicted in FIG. 9C. In this regard, the repositioned transducer-based device representation 901 indicates a repositioning of the transducer-based device (e.g., 200, 300, 400) from its second particular positioning as indicated in FIG. 9C. According to various embodiments, the transducer-based device (e.g., 200, 300, or 400) is repositioned from its second particular positioning after the activation or operation of the first transducer set to deliver the first high voltage pulse set to cause pulsed field ablation of tissue. In FIG. 9E, graphical elements 912 each correspond to a part of a lesion formed in response to the delivery of the first high voltage pulse set, each graphical element 912 having a second visual characteristic set made in response to the determination of the second quality of lesion as per block 806-2, as exemplified by the KEY in FIG. 9E, where the lesion markers 912 have an internal diamond crossing pattern representing the second quality of lesion. According to some embodiments, the graphical elements 912 each correspond to a respective part of a lesion formed in response to the delivery of the first high voltage pulse set, and each graphical element 912 having a second visual characteristic set indicating that respective part of the lesion was determined to have the second quality of lesion. In some embodiments, each graphical element 912 is located in the graphical representation proximate a location of a corresponding one of the transducer graphical elements 905x during the delivery of the first high voltage pulse set. As discussed above, the graphical elements 912 may instead be visually presented as a continuous or overlapping graphical element set, in some embodiments. According to various embodiments, the second visual characteristic set of the graphical elements 912 in FIG. 9E is different than the first visual characteristic set of the graphical elements 912 in FIG. 9D. It is noted that a repositioning of the transducer-based device (e.g., 200, 300, or 400) need not be present in embodiments associated with FIGS. 9D and 9E, and that the graphical elements 912 in FIG. 9D may be generated or displayed while the transducer-based device representation 901 is displayed at the particular location in the graphical representation 900A corresponding to the first particular positioning of the transducer-based device (e.g., 200, 300, or 400), or that the graphical elements 912 in FIG. 9E may be generated or displayed while the transducer-based device representation 901 is displayed at the particular location in the graphical representation 900A corresponding to the second particular positioning of the transducer-based device (e.g., 200, 300, or 400). While FIGS. 9D and 9E continue to show transducer graphical elements as selected after performing the ablation resulting in the indication of lesion markers 912, such transducers may be unselected after performing such ablation in some embodiments.

According to some embodiments, factors other than a degree of transducer-to-tissue separation may be additionally employed to determine a quality of a lesion (per at least block 806) formed or to be formed in response to a delivery of pulsed field ablation energy (e.g., a quality of a lesion formed or to be formed in response to the delivery of the first high voltage pulse set per at least block 802 by the first transducer set). For example, in some embodiments, the data processing device system (e.g., 110, 310) may be configured at least by the program at least to cause (1) the determination (e.g., in accordance with block 806-1) of the first quality of lesion producible in the tissue by the first high voltage pulse set, (2) the determination (e.g., in accordance with block 806-2) of the second quality of lesion producible in the tissue by the first high voltage pulse set, or each of (1) and (2), at least in response to a particular configuration of the first high voltage pulse set. In pulsed field ablation, for example, a determination of an actual or expected quality of the formed or to-be-formed lesion is typically dependent on the amount of PFA energy that is delivered or to-be-delivered to the tissue. In some embodiments, different configurations of the first high voltage pulse set may deliver different amounts of pulsed field ablative energy, and, consequently, different qualities of lesion may be attributed to different configurations of the first high voltage pulse set. Different configurations of a high voltage pulse set for PFA may include, but not be limited to, different amounts of power, different total number of high voltage pulses, different pulse voltages, and different total pulse delivery durations.

In some embodiments, the first high voltage pulse set has a first particular configuration in the first state (e.g., the first state referred to in block 806-1) and has a second particular configuration in the second state (e.g., the second state referred to in block 806-2). According to various embodiments, the second particular configuration of the first high voltage pulse set may be different than the first particular configuration of the first high voltage pulse set. In some embodiments, the data processing device system (e.g., 110, 310) may be configured at least by the program at least to cause the determination (e.g., via block 806-1) of the first quality of lesion producible in the tissue by the first high voltage pulse set at least in response to the first configuration of the first high voltage pulse set. In some embodiments, that data processing device system (e.g., 110, 310) may be configured at least by the program at least to cause the determination (e.g., via block 806-2) of the second quality of lesion producible in the tissue by the first high voltage pulse set at least in response to the second configuration of the first high voltage pulse set. In various embodiments, the determination (e.g., via block 806-1) of the first particular quality of lesion is based at least on (1) the data set (e.g., monitored per at least block 804) indicating the first degree of separation between the second transducer set and the tissue surface, and (2) the first configuration of the first high voltage pulse set. In some embodiments, the determination (e.g., via block 806-2) of the second particular quality of lesion is based at least on (3) the data set indicating the second degree of separation between the second transducer set and the tissue surface, and (4) the second configuration of the first high voltage pulse set. According to some embodiments, the second degree of separation between the second transducer set and the tissue surface is different than the first degree of separation between the second transducer set and the tissue surface, the second configuration of the first high voltage pulse set is different than the first configuration of the high voltage pulse set, and the determined second quality of lesion is different than the determined first quality of lesion. However, it is noted that various sets of different combinations of the degree of separation between the second transducer set and the tissue surface and the particular configuration of the first high voltage pulse set may, in some embodiments, lead to a determination of respective quality of lesion that is the same, or substantially the same for each of the various sets. For example, a second particular quality of lesion determined at least in part on (1) the data set (e.g., monitored per at least block 804) indicating a relatively larger degree of separation between the second transducer set and the tissue surface, and (2) a particular configuration of the first high voltage pulse set configured to deliver a relatively larger amount of PFA energy may be the same or substantially the same as a first particular quality of lesion determined at least in part on (3) the data set indicating a relatively smaller degree of separation between the second transducer set and the tissue surface and (4) a particular configuration of the first high voltage pulse set configured to deliver a relatively smaller amount of PFA energy.

In some embodiments, a particular graphical element set indicating the particular quality of lesion producible in the tissue by the first high voltage pulse set may include a visual characteristic set that changes in accordance with changes in the determined quality of the lesion. For example, in some embodiments, the first graphical element set (e.g., a set of graphical elements 905x in the examples of FIGS. 9B and 9C, and a set graphical elements 912 in the examples of FIGS. 9D and 9E, but other sets may be used in other embodiments) includes a change in at least one visual characteristic to indicate a change in lesion quality from a third quality of lesion to the first quality of lesion during the activation or operation of the associated transducer(s), where the “third” quality of the lesion, in this example, describes the quality of the lesion at a time before the “first” quality of the lesion throughout a duration of time that PFA is being performed by the associated transducer(s). In this example, the third quality of lesion may indicate a relatively lower quality of lesion than the first quality of lesion. For example, the quality of the lesion may, in some embodiments, be dependent on the number of high voltage pulses that are delivered. In particular, pulsed field ablation is cumulative in nature in the sense that the lesion quality generally improves (e.g., increased lesion size or increased lesion depth) as successive pulses are delivered. For example, when considering a case in which the analysis of the data set (e.g., per at least block 806-1) indicates that the first degree of separation between the second transducer set and the tissue surface remains constant, or substantially constant, within some determined or predetermined limits during the activation or operation of the first transducer set to deliver the first high voltage pulse set, successive applications of the pulses in the first high voltage pulse set will improve the lesion quality and this improvement can be communicated to the user (e.g., a health care provider) by changing (e.g., during the activation or operation) the visual characteristic set of the first graphical element set from a third visual characteristic set (e.g., representative of the relatively lower third quality of lesion at a time earlier in the PFA, according to some embodiments) to the first visual characteristic set (e.g., representative of a relatively higher quality of lesion at a time later in the PFA, according to some embodiments). In a similar manner, if the analysis of the data set indicates a change in the degree of separation between the second transducer set and the tissue surface during the activation or operation of the associated transducer(s), the first visual characteristic set of the first graphical element set may change (e.g., during the activation or operation) to reflect a change in lesion quality responsive to the change in the degree of separation. Changes in the visual characteristic set of a particular graphical element set (or a change in the graphical element set itself) indicating the evolution of a particular quality of lesion producible in the tissue is discussed in more detail below in this disclosure at least with respect to FIGS. 8B and 8C.

The use of different configurations of the first high voltage pulse set may be motivated for different reasons according to various embodiments. For example, in some embodiments, information regarding the analysis of the data set (e.g., per at least block 806) indicating a particular degree of separation between the second transducer set and the tissue surface may be communicated, via the input-output device system (e.g., 120, 320), to a user (e.g., a health care practitioner) who may in response to the communicated information, cause a selection of a particular configuration of the first high voltage pulse set (or cause a particular change in a configuration of the first high voltage pulse set) that the user may deem is best suited for use with a particular degree of separation between the second transducer set and the tissue surface.

Different configurations of the first high voltage pulse set (or any other high voltage pulse set) may take different forms, according to various embodiments. For example, in some embodiments, the first configuration of the first high voltage pulse set is configured to deliver a first amount of power, and the second configuration of the first high voltage pulse set is configured to deliver a second amount of power that is different than the first amount of power. In some embodiments, the first configuration of the first high voltage pulse set is configured to deliver a first pulse voltage for each of at least one pulse in the first high voltage pulse set, and the second configuration of the first high voltage pulse set is configured to deliver a second pulse voltage for each of at least one pulse in the first high voltage pulse set, the second pulse voltage different than the first pulse voltage. In some embodiments, the first configuration of the first high voltage pulse set is configured to cause the first high voltage pulse set to have a first total pulse delivery duration, and the second configuration of the first high voltage pulse set is configured to cause the first high voltage pulse set to have a second total pulse delivery duration different than the first total pulse delivery duration. In some embodiments, the first configuration of the first high voltage pulse set is configured to deliver a first total number of high voltage pulses, and the second configuration of the first high voltage pulse set is configured to deliver a second total number of high voltage pulses different than the first total number of high voltage pulses. For example, when delivering a relatively higher number of pulses, the first high voltage pulse set will deliver greater power than when delivering relatively fewer pulses, all else being equal. Referring back to block 808, the display of the first graphical element set, or the second graphical element set, may occur at various times. For example, in some embodiments, display of the first graphical element set or the second graphical element set may occur prior to the activation or operation of the at least the first transducer set to deliver the first high voltage pulse set to cause pulsed field ablation of tissue. In this regard, display of the first graphical element set or the second graphical element set may communicate to the user (e.g., a health care practitioner) a particular quality of the lesion in a predictive manner, according to some embodiments. In some embodiments, display of the first graphical element set, or the second graphical element set occurs during the activation or operation of the at least the first transducer set to deliver the first high voltage pulse set to cause pulsed field ablation of tissue. In this regard, display of the first graphical element set, or the second graphical element set may provide a user (e.g., a health care practitioner) a current or present level of the lesion quality during the activation or operation of the at least the first transducer set to deliver the first high voltage pulse set to cause pulsed field ablation of tissue. In some embodiments, display of the first graphical element set or the second graphical element set may occur after the activation or operation of the at least the first transducer set to deliver the first high voltage pulse set to cause pulsed field ablation of tissue. In this regard, display of the first graphical element set or the second graphical element set may provide a user (e.g., a health care practitioner) a post-activation/operation indicator of the lesion quality.

According to some embodiments, the data processing device system (e.g., 110, 310) may be configured at least by the program at least to cause, via the input-output device system, the monitoring (e.g., via block 804) of the data set at least prior to the activation or operation (e.g., via block 802). In some embodiments, the data processing device system (e.g., 110, 310) may be configured at least by the program at least to cause, via the input-output device system, the monitoring (e.g., via block 804) of the data set at least during the activation or operation (e.g., via block 802). In some embodiments, the data processing device system (e.g., 110, 310) may be configured at least by the program at least to cause, via the input-output device system, the monitoring (e.g., via block 804) of the data set at least after the activation or operation (e.g., via block 802).

Referring to FIG. 8B, which includes according to some embodiments, block 802A, which represents a configuration of the data processing device system (e.g., data processing device system 110 or 310) (e.g., configured according to a program) to cause, via an input-output device system (e.g., input-output device system 120 or 320), activation or operation of at least a first transducer set of a transducer-based device (e.g., 200, 300, or 400) to deliver a first high voltage pulse set to cause pulsed field ablation of tissue. In some embodiments, block 802A of FIG. 8B is similar to or the same as block 802 in FIG. 8A. According to various embodiments, each transducer (e.g., transducer 206, 306, or 406) is configured to deliver high voltage pulses configured to cause pulsed field ablation of tissue. According to some embodiments, the activation or operation of at least a first transducer set is configured to deliver the first high voltage pulse set to cause monopolar PFA. According to some embodiments, the activation or operation of at least a first transducer set is configured to deliver the first high voltage pulse set to cause bipolar PFA. In some embodiments, the pulses in the first high voltage pulse are successively arranged with a constant (regular) pulse-to-pulse spacing.

In FIG. 8B, according to some embodiments, block 804A represents a configuration of the data processing device system (e.g., data processing device system 110 or 310) (e.g., configured according to a program) to cause, via the input-output device system (e.g., input-output device system 120 or 320), monitoring of a data set indicative of proximity between a second transducer set of the transducer-based device (e.g., 200, 300, or 400) and a tissue surface in a bodily cavity. In some embodiments, block 804A of FIG. 8B is the same as, or similar to, block 804 of FIG. 8A, including the corresponding descriptions and possible implementations and characteristics of the first and second transducer sets, except at least that, unlike various embodiments associated with block 804 of FIG. 8A in which the monitored data set is indicative of separation between a second transducer set of the transducer-based device (e.g., 200, 300, or 400) and a tissue surface of a bodily cavity, the monitored first data set associated with block 802A of FIG. 8B is indicative of proximity (e.g., contact or separation) between a second transducer set of the transducer-based device (e.g., 200, 300, or 400) and a tissue surface of a bodily cavity. In this regard, the monitored first data set associated with block 802A is indicative of contact or separation between the second transducer set of the transducer-based device (e.g., 200, 300, or 400) and a tissue surface of a bodily cavity according to various embodiments. Further in this regard, for example, while examples provided above with respect to FIG. 8A pertain to the first transducer set (e.g., referred to at least in block 802) as being associated with transducers (e.g., associated with graphical elements 905x) that exhibit separation (no contact) with tissue, some embodiments associated with FIG. 8B (and some embodiments associated with FIG. 8C discussed in more detail below) include one or more transducers in the first transducer set (e.g., referred to at least in block 802A in FIG. 8B, or at least in block 802B in FIG. 8C discussed in more detail below) that exhibit contact with tissue, that exhibit separation from tissue, or include multiple transducers, some of which exhibit contact with tissue and some of which are separated from tissue.

The first data set indicative of proximity between the second transducer set and the tissue surface per block 804A may be derived in various manners according to various embodiments. For example, proximity including contact between the second transducer set and the tissue surface may be determined by various contact detecting techniques including those described in this disclosure. Proximity including separation between the second transducer set and the tissue surface may be determined by various separation or spacing detecting techniques including those described in this disclosure. Proximity sensors or detectors that detect separation, contact, or both, may take various forms, e.g., as described in this disclosure.

In some embodiments, the input-output device system (e.g., 120, 320) includes a device location tracking system (e.g., 260A, 260B as described as per FIG. 2 or 3 above). In some embodiments, the data processing device system (e.g., 110, 310) is configured at least by the program at least to cause, via the input-output device system (e.g., 120, 320) reception of a location signal set from the device location tracking system, the location signal set indicating a location of at least one transducer in the second transducer set. According to various embodiments, the first data set (e.g., monitored per block 804A) is derived at least in part from the location signal set (for example, as described above in this disclosure). In this regard, proximity indicating contact or separation between the second transducer set and the tissue in the bodily cavity may in some embodiments be determined via the use of a device location tracking system.

According to some embodiments, the data processing device system (e.g., 110, 310) is configured at least by the program at least to cause, via the input-output device system (e.g., 120, 320), the monitoring of the first data set (e.g., per block 804A) at least prior to the delivery of the first high voltage pulse set. In some embodiments, the data processing device system (e.g., 110, 310) is configured at least by the program at least to cause, via the input-output device system (e.g., 120, 320), the monitoring of the first data set at least during the delivery of the first high voltage pulse set. In some embodiments, the data processing device system (e.g., 110, 310) is configured at least by the program at least to cause, via the input-output device system (e.g., 120, 320), the monitoring of the first data set at least after the delivery of the first high voltage pulse set.

In FIG. 8B, according to some embodiments, block 806A represents a configuration of the data processing device system (e.g., data processing device systems 110 or 310) (e.g., according to a program) to cause, based at least on an analysis of the first data set, determination, at least in response to a first state in which the analysis of the first data set is indicative of a first degree of proximity between the second transducer set and the tissue surface, of a first quality of a lesion producible in the tissue by that first high voltage pulse set. Various qualities of lesion are exemplified above in this disclosure.

In FIG. 8B, according to some embodiments, block 808A represents a configuration of the data processing device system (e.g., data processing device systems 110 or 310) (e.g., according to a program) to cause, via the input-output device system (e.g., input-output device systems 120, 320), at least in response to the determination of the first quality of lesion producible in the tissue by the first high voltage pulse set (e.g., per block 806A) display of a first graphical element set indicating the determined first quality of lesion. The first graphical element set may take various forms according to various embodiments. For example, graphical elements, such as graphical elements corresponding to transducers (e.g., graphical elements 905), graphical elements representing tissue regions undergoing, or having undergone ablation (e.g., lesion markers) (e.g., 912), graphical elements indicating locations of ablative energy delivery, graphical elements representing lesions, or any other suitable graphical element set configured to indicate lesion quality may be employed, according to various embodiments.

As described above with respect to FIG. 8A, FIG. 9B shows a graphical representation 900A that includes an envelope 902 providing a three-dimensional representation of a bodily cavity (e.g., a heart atrium) and a transducer-based device graphical representation 901 providing a three-dimensional representation of a transducer-based device (e.g., 200, 300, or 400), the transducer-based device graphical representation 901 including a plurality of transducer graphical elements 905 representative of various transducers of the transducer-based device. According to various embodiments, in FIG. 9B, a first graphical element set made up of or including transducer graphical elements 905x is displayed according to block 808A of FIG. 8B. Such transducer graphical elements 905x may indicate the determined first quality of lesion, as indicated in the “1ST LESION QUALITY” entry in the KEY in FIG. 9B, and, as described above with respect to FIG. 8A, in a state in which the monitored degree of proximity per block 806A indicates separation between the transducers associated with transducer graphical elements 905x and the tissue surface (e.g., as per FIG. 9A). In this regard, according to various embodiments, the first quality of lesion associated with the first graphical element set made up of or including graphical elements 905x is based on an analysis of a first data set (e.g., per block 806A) that is indicative of a first degree of proximity between the second transducer set and the tissue surface in a state in which the first degree of proximity indicates a degree of separation (i.e., no contact) between the second transducer set (e.g., the transducers corresponding to transducer graphical elements 905x in this example) and the tissue surface.

Other first graphical element sets may also be displayed according to various embodiments associated with block 808A of FIG. 8B. For example, in FIG. 9B a first graphical element set made up of or including transducer graphical elements 905a, 905b, and 905c is displayed according to block 808A of FIG. 8B. Such transducer graphical elements 905a, 905b, and 905c may indicate the determined first quality of lesion, as indicated by the “OTHER LESION QUALITY” entry in the KEY in FIG. 9B, in a state in which the monitored degree of proximity per block 806A indicates a degree of contact between the second transducer set (e.g., the transducers corresponding to transducer graphical elements 905a, 905b, and 905c in this example) and the tissue surface. In this regard, in some embodiments in which the second transducer set is the first transducer set referred to in block 802A, such first transducer set may include the transducers associated with graphical elements 905a, 905b, and 905c that exhibit tissue contact (e.g., as per FIG. 9A). According to some embodiments, different first graphical element sets are displayed concurrently. For example, in FIG. 9B, the first graphical element set made up of or including graphical elements 905x and the first graphical element set made up of or including graphical elements 905a, 905b and 905c are displayed concurrently, in some embodiments. In this regard, in some embodiments in which the second transducer set is the first transducer set referred to in block 802A, such first transducer set may include the transducers associated with graphical elements 905a, 905b, 905c, and 905x, some of which exhibit tissue contact and some of which are separated from tissue.

In FIG. 8B, according to some embodiments, block 810A represents a configuration of the data processing device system (e.g., data processing device systems 110) (e.g., at least by the program) to cause, via the input-output device system (e.g., 110, 310), second operation of at least a third transducer set (e.g., a set including one or more transducers 206, 306, or 406) of the transducer-based device (e.g., 200, 300, or 400) to deliver a second high voltage pulse set to cause pulsed field ablation of the tissue. According to various embodiments, the delivery of the second high voltage pulse set occurs after the delivery of the first high voltage pulse set per block 802A. According to various embodiments, the third transducer set of the transducer-based device (e.g., 200, 300, or 400) is the first transducer set of the transducer-based device, such that, for example, the same transducer(s) that deliver the first high voltage pulse set per block 802A also deliver the second high voltage pulse set per block 810A. According to various embodiments, the third transducer set of the transducer-based device (e.g., 200, 300, or 400) is other than the first transducer set of the transducer-based device, such that, for example, different transducers deliver the first and second high voltage pulse sets.

In FIG. 8B, according to some embodiments, block 812A represents a configuration of the data processing device system (e.g., data processing device systems 110) (e.g., at least by the program) to cause determination of a second quality of the lesion producible in the tissue by the delivery of the second high voltage pulse set. In FIG. 8B, according to some embodiments, block 814A represents a configuration of the data processing device system (e.g., data processing device systems 110) (e.g., at least by the program) to cause, via the input-output device system (e.g., 110, 310) and at least in response to determination of the second quality of lesion producible in the tissue per block 812A, display of a second graphical element set indicating the determined second quality of the lesion. According to various embodiments, the second quality of lesion (e.g., indicated by the second graphical element set as per block 814A, in some embodiments) indicates a greater degree of tissue damage as compared to the first quality of the lesion (e.g., indicated by the first graphical element set as per block 808A, in some embodiments). According to various embodiments, the second quality of lesion (e.g., indicated by the second graphical element set as per block 814A, in some embodiments) indicates a greater degree of lesion size as compared to the first quality of the lesion (e.g., indicated by the first graphical element set as per block 808A, in some embodiments). According to various embodiments, the second quality of lesion (e.g., indicated by the second graphical element set as per block 814A, in some embodiments) indicates a greater degree of lesion depth as compared to the first quality of the lesion (e.g., indicated by the first graphical element set as per block 808A, in some embodiments). According to various embodiments, the second quality of the lesion producible in the tissue indicates a cumulative effect on the tissue as a result of at least delivery of the first high voltage pulse set and the second high voltage pulse set. In this regard, the determined second quality of the lesion may be an indication of an actual cumulative effect, e.g., at least in a case in which the first and second high voltage pulse sets have been delivered or in which the first high voltage pulse set has been delivered and the second high voltage pulse set is being delivered, or the determined second quality of the lesion may be an indication of a predicted cumulative effect, e.g., at least in a case in which the first high voltage pulse set has been delivered, but the second high voltage pulse set has not yet been delivered or has not yet been completely delivered, such that the determined second lesion quality predicts the cumulative effect on the tissue if the second high voltage pulse set is delivered or completely delivered, according to some embodiments.

Determining a cumulative effect of multiple ablations on a region of tissue varies depending on the ablation technique used. In thermal ablation (e.g., RF ablation), energy is delivered in order to set up an elevated temperature distribution within the tissue, and depending on the duration of energy application, the lesion quality may be governed primarily by the depth at which an approximately steady-state ablation-level temperature has been achieved. Accordingly, the ablation lesion increases in size rapidly on initial energy application, but additional lesion depth increase upon reaching a near steady-state temperature profile may be quite minimal due to the high temperature dependence of the rate of thermal damage accumulation. However, if the thermal ablation is stopped after achieving the steady-state ablation-temperature condition, and then the tissue is allowed to cool, and then thermal ablation is restarted after the cool down, the thermal field required for ablation would essentially be required to be regenerated from scratch (e.g., from baseline temperature). The temperature of the tissue from this second (i.e., repeated) thermal ablation increases at basically the same rate that occurred during the first thermal ablation in order to again achieve the steady-state ablation-temperature condition. However, because the second application does not result in any further penetration of the temperature field (because it is at or near steady-state), then the second application will achieve little by way of increased lesion extent as compared to the first application, where the first application had also reached a near steady-state temperature profile. Accordingly, in the context of thermal ablation, when determining the cumulative effect of a second ablation of a tissue region after having performed a first ablation of the tissue region that reached steady-state, the second ablation may be considered to not have any additional clinically relevant cumulative effect on the lesion quality at the conclusion of the first ablation, in some embodiments. If the first ablation did not reach the steady-state temperature, however, then the second ablation may provide a clinically relevant cumulative effect on the lesion quality. Of course, if there is no delay or pause between the first and second thermal ablations, there would be no ramp up period, and a cumulative effect may be determined as if the first and second thermal ablations were a single uninterrupted thermal ablation although at steady state this increase may not be clinically relevant.

In pulsed field ablation, however, each delivered high voltage pulse has some probability of opening a small permanent hole in the cell membrane. It is these permanent holes that lead to cell death which causes the ablation mechanism in PFA. It is noted that once these permanent holes are formed, the delivery of additional high voltage pulses provide more opportunities for creating further permanent holes, which can enhance the ablation lesion quality (e.g., increase lesion size, or depth, or both). In some contexts, it may be considered that, once a first set of cellular membrane holes is formed in the tissue during PFA by a first high voltage pulse set, the first set of cellular membrane holes may remain permanently, and, consequently, the delivery of a second high voltage pulse set may create a second additional set of cellular holes generally regardless of how much time has elapsed between the delivery of the first high voltage pulse set and the second high voltage pulse set. Consequently, a delay or pause between ablative high voltage pulse sets in PFA, as discussed above with respect to thermal ablation, generally speaking need not be considered when determining a cumulative effect of multiple ablations using PFA.

The second graphical element set displayed in accordance with block 814A may take various forms, according to various embodiments. For example, graphical elements such as graphical elements corresponding to transducers (e.g., graphical elements 905), graphical elements representing tissue regions undergoing, or having undergone ablation (e.g., lesion markers) (e.g., 912), graphical elements indicating locations of ablative energy delivery, graphical elements representing lesions, or any other suitable graphical element set configured to indicate lesion quality may be employed according to various embodiments. According to various embodiments, the second graphical element set is the first graphical element set but includes a change in at least one visual characteristic to indicate a change in lesion quality from the first quality of the lesion due to the delivery of the second high voltage pulse set. According to various embodiments associated with FIG. 9F, a second graphical element set indicating the determined second quality of the lesion is displayed according to block 814A of FIG. 8B. In this regard, according to some embodiments, a second graphical element set made up of or including transducer graphical elements 905x is displayed in FIG. 9F according to block 814A of FIG. 8B. In some embodiments, a visual characteristic set of the second graphical element set made up of or including graphical elements 905x has a visual characteristic set indicating the determined second quality of lesion (as indicated in the KEY in FIG. 9F, where graphical elements 905x have the “1ST ENHANCED LESION QUALITY” dotted pattern, as differentiated by the “1ST LESION QUALITY” KEY entry shown in FIG. 9B). According to various embodiments, the second graphical element set made up of or including transducer graphical elements 905x that is displayed according to block 814A in FIG. 9F includes the same graphical elements 905x that are included in the first graphical element set displayed according to block 808A in FIG. 9B, but a visual characteristic set of the first graphical element set of graphical elements 905x in FIG. 9B has changed to produce the second graphical element set of graphical elements 905x in FIG. 9F, which have the different “1ST ENHANCED LESION QUALITY” dotted pattern in the state of FIG. 9F compared to the “1ST LESION QUALITY” entry in the KEY of FIG. 9B. According to various embodiments, a visual characteristic set of the second graphical element set of graphical elements 905x indicates a second quality of the lesion producible in the tissue as a cumulative effect on the tissue as a result of at least delivery of the first high voltage pulse set per block 802A of FIG. 8B and delivery of the second high voltage pulse set per block 810A of FIG. 8B.

Other second graphical element sets are displayed in FIG. 9F according to some embodiments. For example, in FIG. 9F, a second graphical element set made up of or including transducer graphical elements 905a, 905b, and 905c is displayed according to block 814A of FIG. 8B. According to some embodiments, FIG. 9F includes the same graphical elements 905a, 905b, 905c that are included in the first graphical element set according to some embodiments displayed according to block 808A in FIG. 9B, but a visual characteristic set of the first graphical element set of graphical elements 905a, 905b, 905c in FIG. 9B has changed to produce the second graphical element set of graphical elements 905a, 905b, 905c in FIG. 9F. As shown in FIG. 9F, a visual characteristic set of a second graphical element set made up of or including graphical elements 905a, 905b and 905c have a visual characteristic set indicating a second quality of lesion (as indicated in the KEY in FIG. 9B, where graphical elements 905a, 905b, and 905c have the “2ND ENHANCED LESION QUALITY” 90-degree crossing pattern, as differentiated by the “OTHER LESION QUALITY” entry in the KEY of FIG. 9B) determined as per block 812A of FIG. 8B.

According to various embodiments, a visual characteristic set of the second graphical element set of graphical elements 905a, 905b, and 905c indicates a second quality of the lesion producible in the tissue as cumulative effect on the tissue as a result of at least delivery of the first high voltage pulse set per block 802A of FIG. 8B and delivery of the second high voltage pulse set per block 810A of FIG. 8B. According to various embodiments, the first high voltage pulse set and the second high voltage pulse set form part of an uninterrupted high voltage pulse train. In some embodiments, the first high voltage pulse set and the second high voltage pulse set form successive sets in the uninterrupted high voltage pulse train. For example, with reference to various embodiments described above with respect to FIGS. 9B and 9F, the selected transducers corresponding to at least graphical elements 905x may deliver both the first high voltage pulse set and the second high voltage pulse set as part of (e.g., successive sets of) an uninterrupted high voltage pulse train. In various embodiments, successive pulses in the uninterrupted high voltage pulse train are temporally spaced with a uniform, or substantially uniform, pulse-to-pulse spacing. In various embodiments, the data processing device system (e.g., 110, 310) may initiate delivery, via the input-output device system (e.g., 120, 320), of an uninterrupted high voltage pulse train in accordance with initiation of high voltage pulse train delivery program instructions, and the data processing device system (e.g., 110, 310) may cause cessation of delivery, via the input-output device system (e.g., 120, 320), of the uninterrupted high voltage pulse train in accordance with cessation of high voltage pulse train delivery program instructions.

According to various embodiments, the third transducer set (e.g., that delivers the second high voltage pulse train per block 810A) of the transducer-based device (e.g., 200, 300, or 400) is the first transducer set (e.g., that delivers the first high voltage pulse train per block 802A) of the transducer-based device (e.g., 200, 300, or 400). In various embodiments, the same transducers (e.g., 206, 306, or 406) of the transducer-based device (e.g., 200, 300, or 400) may be employed to deliver both the first high voltage pulse set and the second high voltage pulse sets. For example, in various embodiments described above with respect to FIGS. 9B and 9F, the transducers (e.g., 206, 306, or 406) corresponding to graphical elements 905x may deliver both the first high voltage pulse set and the second high voltage pulse set of a first particular group of first and second high voltage pulse sets according to some embodiments, and the transducers (e.g., 206, 306, or 406) corresponding to graphical elements 905a, 905b, and 905c may deliver both the first high voltage pulse set and the second high voltage pulse set of a second particular group of first and second high voltage pulse sets, according to some embodiments.

In FIG. 8B, according to some embodiments, block 811A represents a particularly optional (indicated by the broken-line outline of block 811A in FIG. 8B), but beneficial in at least some contexts, configuration of the data processing device system (e.g., data processing device system 110 or 310) (e.g., configured according to a program) to cause, via the input-output device system (e.g., 120, 320), monitoring of a second data set indicative of proximity between a fourth transducer set of the transducer-based device (e.g., 200, 300, or 400) and the tissue surface in the bodily cavity. In this regard, such monitoring may be the same or similar as described above with respect to block 804A, but for monitoring a second data set indicative of proximity between a fourth transducer set of the transducer-based device (e.g., 200, 300, or 400) and the tissue surface in the bodily cavity. According to various embodiments, the data processing device system (e.g., 110, 310) may be configured at least by the program at least to cause the determination of the second quality of the lesion producible in the tissue by the second high voltage pulse set per block 812A based at least on an analysis of the second data set. In this regard, although not stated inside block 812A, some embodiments associated with block 812A may include determining the second quality of the lesion indicating the cumulative effect of the first and second high voltage pulse sets not only in response to the activation or operation of the third transducer set to deliver the second high voltage pulse set, but also based at least on an analysis of the second data set monitored per block 811A. According to some embodiments, the determination of the second quality of the lesion producible in the tissue by the second high voltage pulse set may be made at least in response to a second state in which the analysis of the second data set is indicative of a second degree of proximity between the fourth transducer set and the tissue surface. In various embodiments, this second degree of proximity may be the same or different than the first degree of proximity indicated in block 806A. In some embodiments, the fourth transducer set is the third transducer set, such that the same transducer set that delivers the second high voltage pulse set per block 810A also has its proximity to tissue monitored per block 811A.

In some embodiments, the fourth transducer set of the transducer-based device (e.g., 200, 300, or 400) is other than the second transducer set of the transducer-based device (e.g., 200, 300, or 400). In some embodiments, the fourth transducer set of the transducer-based device (e.g., 200, 300, or 400) is the second transducer set (e.g., whose proximity is monitored per block 804A) of the transducer-based device (e.g., 200, 300, or 400). For example, according to some embodiments, a same particular transducer set of the transducer-based device (e.g., 200, 300, or 400) may be utilized during both the monitoring of the first degree of proximity between the second transducer set and the tissue surface (e.g., as per block 806A), and the monitoring of the second degree of proximity between the fourth transducer set and the tissue surface. According to various embodiments, the determination of the first quality of the lesion (e.g., per block 806A) producible in the tissue by the first high voltage pulse set (e.g., delivered per block 802A) is made at least in response to the first state in which the analysis (e.g., per block 806A) of the first data set (e.g., monitored per block 804A) is indicative of a first degree of proximity between the at least the part of the second transducer set and the tissue surface, and the determination of the second quality of the lesion (e.g., per block 812A) producible in the tissue by the second high voltage pulse set (e.g., delivered per block 810A) is made at least in response to the second state in which the analysis (e.g., which may be included as part of block 812A) of the second data set (e.g., monitored per block 811A) is indicative of a second degree of proximity between the fourth transducer set and the tissue surface.

In some embodiments, the third transducer set (e.g., which delivers the second high voltage pulse set per block 810A) is the first transducer set (e.g., which delivers the first high voltage pulse set per block 802A). In some embodiments, the fourth transducer set (e.g., whose proximity in some embodiments is monitored via the second data set referred to above) is the second transducer set (e.g., whose proximity in some embodiments is monitored via the first data set per block 804A). In some embodiments, each of the second transducer set and the third transducer set is the first transducer set. In some embodiments, each of the second transducer set, the third transducer set, and the fourth transducer set is the first transducer set, such that the same transducer set delivers the first high voltage pulse set per block 802A, delivers the second high voltage pulse set per block 810A, and has its proximity to tissue monitored via the first data set per block 804A and again via the second data set as discussed above. According to various embodiments, at least one transducer in the second transducer set, the fourth transducer set, or each of the second transducer set and the fourth transducer set may be a transducer configured to deliver ablative energy (e.g., PFA energy, in some embodiments). In some embodiments, at least one transducer in the first transducer set, the second transducer set, the third transducer set, or the fourth transducer set may be configured to perform a function other than, or in addition to, a delivery of ablative energy (e.g., a delivery of PFA energy, in some embodiments). For example, the at least one transducer may be configured to sense various information including electrophysiological information, electrical information including impedance information, and temperature information. In some embodiments, (a) the second degree of proximity between the fourth transducer set and the tissue surface indicates contact between at least one transducer in the fourth transducer set and the tissue surface, (b) the first degree of proximity between the second transducer set and the tissue surface indicates contact between at least one transducer in the second transducer set and the tissue surface, or each of (a) and (b). In some embodiments, (a) the second degree of proximity between the fourth transducer set and the tissue surface indicates separation between at least one transducer in the fourth transducer set and the tissue surface, (b) the first degree of proximity between the second transducer set and the tissue surface indicates separation between at least one transducer in the second transducer set and the tissue surface, or each of (a) and (b).

According to some embodiments, the second degree of proximity between the fourth transducer set and the tissue surface is the same as the first degree of proximity between the second transducer set and the tissue surface. In some embodiments, the first quality of the lesion producible in the tissue by the first high voltage pulse set is determined (e.g., per block 806A) at least in response to the first state in which the analysis of the first data set is indicative of the first degree of proximity between the second transducer set and the tissue surface, and the second quality of the lesion producible in the tissue by the second high voltage pulse set is determined (e.g., per some embodiments of block 812A) at least in response to the second state in which the analysis of the second data set is indicative of the second degree of proximity between the fourth transducer set and the tissue surface, the second degree of proximity between the fourth transducer set and the tissue surface being the same as the first degree of proximity between the second transducer set and the tissue surface, in some embodiments. In some embodiments, the fourth transducer set is the second transducer set. According to some embodiments associated with FIGS. 9B and 9F, the first graphical element set of transducer graphical elements 905x displayed in FIG. 9B may be indicative of the first quality of the lesion determined at least in response to the first state in which the first data set is indicative of the first degree of proximity, and the second graphical element set of transducer graphical elements 905x displayed in FIG. 9F may be indicative of the second quality of the lesion determined at least in response to the second state in which the second data set is indicative of the second degree of proximity, the second degree of proximity being the same as the first degree of proximity In some embodiments, the first degree of proximity between the second transducer set and the tissue surface and the second degree of proximity between the fourth transducer set and the tissue surface are a same degree of separation between respective ones of the second transducer set and the fourth transducer set and the tissue surface. In some embodiments, the first degree of proximity between the second transducer set and the tissue surface and the second degree of proximity between the fourth transducer set and the tissue surface are a same degree of contact between respective ones of the second transducer set and the fourth transducer set and the tissue surface. In some embodiments, the second degree of proximity between the fourth transducer set and the tissue surface is different than the first degree of proximity between the second transducer set and the tissue surface. In some embodiments, the fourth transducer set is the second transducer set. Differences between the first degree of proximity and the second degree of proximity may take different forms in various embodiments. For example, in some embodiments, one of the first degree of proximity between the second transducer set and the tissue surface and the second degree of proximity between the fourth transducer set and the tissue surface indicates separation from the tissue surface, while the other of the first degree of proximity between the second transducer set and the tissue surface and the second degree of proximity between the fourth transducer set and the tissue surface indicates contact with the tissue surface. In some embodiments, the first degree of proximity between the second transducer set and the tissue surface indicates a first particular degree of contact with the tissue surface, and the second degree of proximity between the fourth transducer set and the tissue surface indicates a second particular degree of contact with the tissue surface, the second particular degree of contact with the tissue surface different than the first particular degree of contact with the tissue surface. In some embodiments, the first degree of proximity between the second transducer set and the tissue surface indicates a first particular degree of separation with the tissue surface, and the second degree of proximity between the fourth transducer set and the tissue surface indicates a second particular degree of separation with the tissue surface, the second particular degree of separation with the tissue surface different than the first particular degree of separation with the tissue surface. The quality of a lesion formed by pulsed field ablation is typically dependent on various factors. As mentioned above in this disclosure, the quality of the lesion may, in some embodiments, be dependent on the number of high voltage pulses that are delivered. In particular, pulsed field ablation may be considered cumulative in nature in the sense that the lesion quality generally improves (e.g., increased lesion size or increased lesion depth) as successive pulses of one or more high voltage pulse trains are delivered. The degree lesion quality improvement may also be, in some embodiments, dependent on the proximity of various transducers (e.g., the transducers delivering the PFA energy) to each other and to the tissue surface. Generally, proximity conditions involving contact with the tissue surface during a delivery of particular pulsed field ablation energy will result in a lesion of relatively greater quality than a quality of lesion formed as a result of the delivery of the particular pulsed field ablation energy under proximity conditions involving separation from the tissue surface, all else being equal. Generally, proximity conditions involving relatively higher degrees of contact with the tissue surface during a delivery of particular pulsed field ablation energy will result in a lesion of relatively greater quality than a quality of lesion formed as a result of the delivery of the particular pulsed field ablation energy under proximity conditions involving relatively lower degrees of contact with the tissue surface. Generally, proximity conditions involving relatively lower degrees of separation with the tissue surface during a delivery of particular pulsed field ablation energy will result in a lesion of relatively greater quality than a quality of lesion formed as a result of the delivery of the particular pulsed field ablation energy under proximity conditions involving relatively higher degrees of separation with the tissue surface.

According to various embodiments, the first quality of lesion producible in the tissue may be determined at least in response to the first state (e.g., referred to in block 806A) in which the analysis of the first data set is indicative of the first degree of proximity between the second transducer set and the tissue surface, and the second quality of lesion producible in the tissue may be determined at least in response to the second state in which the analysis of the second data set (e.g., monitored per block 811A) is indicative of the second degree of proximity between the fourth transducer set and the tissue surface. In some embodiments, the second degree of proximity is equal to, or is deemed to be within some predetermined or determined range of the first degree of proximity to have a relatively negligible difference. In some embodiments in which the fourth transducer set and the second transducer set are the same, and in which the particular degree of proximity determined from the analysis of the data sets monitored per blocks 804A and 811A is the same or within a threshold corresponding to a relatively negligible change, the second quality of the lesion may, in some embodiments, be determined per some embodiments of block 812A based at least on each of (a) information related to the number of high voltage pulses delivered or intended to be delivered by the first high voltage pulse set and (b) information related to the number of high voltage pulses delivered or intended to be delivered by the second high voltage pulse set, in some embodiments. In some embodiments, the second quality of the lesion may be determined per some embodiments of block 812A based at least on a combination of (a) and (b), which, in some embodiments, may include a determination of the resulting second lesion quality based on the same particular degree of proximity that exists in each of the first and second states. In this regard, in various embodiments, the second quality of lesion may indicate an enhanced degree of quality as compared to the first quality of the lesion. For example, a particular quality of the lesion determined in accordance with a delivery of the first high voltage pulse set under a particular set of proximity conditions generally improves with an additional delivery of the second high voltage pulse set under the same particular set of proximity conditions.

In some embodiments, the second degree of proximity may be different, or may be deemed different if outside a determined or predetermined range corresponding to an accepted amount of equivalence from the first degree of proximity. For example, at least in some embodiments in which the fourth transducer set (e.g., whose proximity is monitored per block 811A) is the second transducer set (e.g., whose proximity is monitored per block 804A), the particular degree of proximity between the second transducer set may be different in the second state than it was in the first state. In at least some embodiments in which the first and second degrees of proximity are different or sufficiently different to need to be considered in determining effects on cumulative lesion quality, in some embodiments, the second quality of the lesion may, in some embodiments, be determined per some embodiments of block 812A based at least on each of (a) information derived from the number of high voltage pulses deliverable by the first high voltage pulse set and the first degree of proximity, and (b) information derived from the number of high voltage pulses deliverable by the second high voltage pulse set and the second degree of proximity In some embodiments, (a) indicates an effective number of high voltage pulses deliverable by the first high voltage pulse set as the actual number of high voltage pulses deliverable by the first high voltage pulse set adjusted in accordance with the first degree of proximity In some embodiments, (b) indicates an effective number of high voltage pulses deliverable by the second high voltage pulse set as the actual number of high voltage pulses deliverable by the second high voltage pulse set adjusted in accordance with the second degree of proximity. The second quality of the lesion may, in some embodiments, be determined based at least on a sum of (c) information derived from the power delivered by the first transducer set (or power delivered to the first transducer set, e.g., supply power) and the first degree of proximity, and (d) information derived from the power delivered from the third transducer set (or power delivered to the third transducer set, e.g., supply power) and the second degree of proximity In some embodiments, (c) indicates an effective power (e.g., an effective power delivered by the first transducer set or an effective power supplied to the first transducer set) as the actual power delivered by or supplied to the first transducer set adjusted in accordance with the first degree of proximity In some embodiments, (d) indicates an effective power (e.g., an effective power delivered by the third transducer set or an effective power supplied to the third transducer set) as the actual power delivered by or supplied to the third transducer set adjusted in accordance with the second degree of proximity. The present inventors have derived several approaches for determining the lesion quality for ablation procedures, such as PFA. Various principles adopted by the inventors in the derivation of these approaches included:
(a) PFA pulses could be delivered in different sets separated in time without affecting the result. For example, a delivery of one set of 200 pulses produces the same lesion quality (e.g., lesion depth) as two sets of 100 pulse delivered separately in time (factors such as proximity and transducer placement set aside).
(b) A minimum field intensity (voltage or voltage gradient) required to cause lesions.
(c) Lesion quality (e.g., lesion depth) increases monotonically with a decreasing rate with additional pulses, increasing approximately as an exponential approaching an asymptotic lesion depth where additional pulses no longer increase the lesion depth because the voltage gradient is too small (factors such as proximity and transducer placement set aside).
(d) The approaches account for various pulse sets delivered under conditions involving different degrees of transducer-to-tissue proximity
(e) Cases of proximity that involve separation from the tissue involve a drop in lesion depth (or penetration of the voltage gradient) by an amount related to the separation or displacement. For example, in some embodiments, the drop in lesion depth (or penetration of the voltage gradient) may be modeled as the same amount as the separation or displacement. In theory, displacement of a PFA transducer away from the tissue results in a drop in lesion depth that is proportional, but only exactly where the conductivities of the tissue and medium are the same. If the conductivities differ, one may expect that there may be some error in this proportionality since the current will preferentially go through the more conductive medium (though the effect on lesions is more subtle since it is based on the resulting voltage gradient). However, the difference between blood and tissue conductivity may not be so great. The inventors believe that some in the art have indicated that there is a change from about 0.7 to 0.4 S/m in the conductivity of tissue as compared to the conductivity of blood in animal studies. However, in the experience of the inventors, these numbers do not appear to match in humans (where the variability of the current output does not vary as much as expected based on contact with tissue or blood in circumstances where these conductivity differences are substantially true. Therefore, in some embodiments, the drop in lesion depth (or penetration of the voltage gradient) may be modeled as the same amount as the separation. In some embodiments, the drop in lesion depth (or penetration of the voltage gradient) may be modeled as a different amount from the separation or displacement.
(f) In cases of proximity that involve different degrees of contact measurements of transducer-to-tissue contact, such cases may be converted to displacement estimates. This conversion may be accomplished by the use of various experiments where lesions that are made at specific degrees of contact or displacement are assessed to determine a reliable depth relationship. It is noted that, in some embodiments, these contact values may be approximations and may need to be treated coarsely (e.g., converting contact measures of good, modest, or poor degrees of contact to displacement values). Alternatively, the contact measures may be experimentally converted to displacement measures which are then applied to alter the calculation of lesion depths.

A first approach for determining the lesion quality for ablation procedures is voltage gradient based, the voltage gradient created by the delivery of PFA energy. According to some embodiments associated with this approach, an estimate of the voltage gradient with distance from the electrode is made. This estimate may be made in various ways. For example, in some embodiments, the estimate may be made using a lookup table generated from direct measurements in bench testing. In some embodiments, the estimate may be made from a finite element analysis or equivalent physical computer model. In some embodiments, the estimate may be made using an approximation voltage field with a closed form solution (e.g., such as for a spherical electrode or a superposition of voltages from two spherical electrodes representing positive and negative voltages). According to various embodiments, the first approach may include converting an employed metric of separation or contact or degree of contact into an estimate of the displacement of the PFA transducer from the tissue surface. For example, if the voltage gradient value is known at a distance of 5 mm from a PFA transducer (e.g., PFA electrode), then for a displacement of zero mm from the tissue, the “5 mm” voltage gradient value is employed. However, if the displacement is 2 mm away from the tissue, a voltage gradient value at 7 mm from the PFA transducer is employed because the 5 mm tissue depth is an additional 2 mm away from the PFA transducer. According to various embodiments, the estimated displacement is factored into the voltage gradient estimates. According to various embodiments, the first approach may include calculating a damage integral “Q” at multiple depths in the tissue. In this regard, according to some embodiments, it may be assumed that a lesion forms when the damage integral Q exceeds some critical threshold (e.g., Q>1). According to some embodiments, the first approach may include estimating the lesion depth by interpolation of the damage integral Q (for example, using techniques using binary search, regularly sampled depths, or other estimation algorithms). According to various embodiments, a lesion quality may correspond to, or be derived from, the estimated lesion depth. Relationship (1) may be employed according to some embodiments to derive or estimate a damage integral that produces the expected desired behavior that would correspond to the voltage gradient estimated at each assessed depth:

Ω(d)=Σᵢ₌₁^{i=imax}A(max(∥∇V_{d,i,z}∥−B),0)^{C} (1)

Where:
d is the depth level at which the damage integral is being evaluated;
Q is the damage integral;
i is the pulse number;
z is the PFA transducer distance from the tissue surface;
V is the voltage (of depth d and for pulse number i);
A is a constant that relates to the threshold for lesion formation;
B is a constant that relates to the minimum voltage gradient required to permit lesion formation; and
C is a constant that affects the sensitivity of the damage integral rate to the voltage gradient.

It is noted that relationship (1) is dependent on the number of pulses that are delivered with a greater value of the damage integral Q with greater numbers of pulses.

A second approach for determining the lesion quality for ablation procedures is voltage based, the voltage associated with the delivery of PFA energy. According to some embodiments, a voltage-based approach does not directly use estimates of the voltage gradient with displacement under the PFA transducer. In some embodiments, after each delivered pulse, the lesion depth is computed to be increased slightly based on a function of the maximum possible depth (as affected by voltage and subtracting any displacement representing separation). According to some embodiments, the computed incremental depth may then be modified based on how far the lesion already extends (e.g., growing slower for well-developed lesions, but quickly during the initial formation of the lesions).

Relationship (2) may be employed according to some embodiments to derive or estimate a lesion depth based on the voltage-based approach:

Dᵢ₊₁=Dᵢ+A{max[B(Vᵢ−Vₜₕᵣ)^{C}−Dᵢ−zᵢ,0]} (2)

Where:
Dᵢ is the lesion depth after pulse i;
V is the voltage associated with the delivering PFA transducer;
Vₜₕᵣ is the threshold electrode voltage for lesion formation;
A is a constant that affects the dependence of lesion depth increase to the number of pulses applied;
B is a constant affecting the relationship between voltage and maximum lesion depth;
C is a constant affecting the power relationship for maximum potential lesion depth based on voltage; and
zᵢ is the displacement of the electrode from the tissue surface for pulse i.

According to various embodiments, a lesion score may correspond to or be derived from the calculated lesion depth. It is noted that relationship (2) is dependent on the number of pulses that are delivered (e.g., a greater value of the damage integral Q is associated with a greater numbers of pulses). Unlike relationship (1), relationship (2) allows for the lesion depth to be calculated directly and an interpolation and/or binary search method is not required. Referring to block 812A of FIG. 8B, the cumulative effect on the tissue caused as a result of at least delivery of the first high voltage pulse set and the second high voltage pulse set may, in some embodiments, be a predicted cumulative effect (for example, predicted according to some embodiments via the use of relationship (1) or relationship (2) described above). In some embodiments, the cumulative effect of the tissue is a measured cumulative effect.

Monitoring of the second data set (e.g., per block 811A) may occur at various times, according to some embodiments. In some embodiments, the data processing device system (e.g., 110, 310) is configured at least by the program at least to cause, via the input-output device system (120, 320), the monitoring of the second data set (e.g., per block 811A) at least prior to the delivery of the second high voltage pulse set (e.g., per block 810A). In some embodiments, the data processing device system (e.g., 110, 310) may be configured at least by the program at least to cause the monitoring of the second data set (e.g., per block 811A) to occur at least in part during the delivery of the first high voltage pulse set (e.g., per block 802A). In some embodiments, the data processing device system (e.g., 110, 310) may be configured at least by the program at least to cause the monitoring of the second data set (e.g., per block 811A) to occur at least in part after the delivery of the first high voltage pulse set (e.g., per block 802A). For example, in some embodiments, determination of the second quality of the lesion (e.g., per block 812A) may be made in a predictive or expected manner from a determination of proximity between the fourth transducer set and the tissue surface (e.g., based on the second data set monitored per block 811A) made prior to the actual delivery of the second high voltage set (e.g., per block 810A).

In some embodiments, the data processing device system (e.g., 110, 310) is configured at least by the program at least to cause, via the input-output device system (e.g., 120, 320), the monitoring of the second data set (e.g., per block 811A) during the delivery of the second high voltage pulse set (e.g., per block 810A). For example, in some embodiments, determination of the second quality of the lesion may be made based at least on a measured determination of proximity between the fourth transducer set and the tissue surface made during the actual delivery of the second high voltage set. In some embodiments, the data processing device system (e.g., 110, 310) may be configured at least by the program at least to cause, via the input-output device system (e.g., 120, 320), the monitoring of the second data set (e.g., per block 811A) at least after the delivery of the second high voltage pulse set (e.g., per block 810A). For example, in some embodiments, determination of the second quality of the lesion (e.g., per block 812A) producible in the tissue by the second high voltage pulse may be based at least on an analysis of the second data set that was acquired or monitored (e.g., per block 811A) at least after the completion of the delivery of the second high voltage pulse set (e.g., per block 810A). For instance, the acquisition and monitoring of the second data set during the delivery of the second high voltage pulse set may be difficult in some embodiments due to electrical interference effects caused by the delivering of the pulses, and the acquisition and monitoring of the second data set after the delivery of the second high voltage pulse set may be employed to mitigate these effects, in some embodiments.

In some embodiments, the first high voltage pulse set and the second high voltage form part of an uninterrupted high voltage pulse train. In some embodiments, the first high voltage pulse set and the second high voltage pulse set are successive high voltage pulse sets in the uninterrupted high voltage pulse train (e.g., the high voltage pulse train does not include any high voltage pulses or high voltage pulse sets between the first high voltage pulse set and the second high voltage pulse set). In some embodiments, the first high voltage pulse set and the second high voltage pulse set are not successive high voltage pulse sets in the uninterrupted high voltage pulse train (e.g., the high voltage pulse train includes at least one high voltage pulse set between the first high voltage pulse set and the second high voltage pulse set). For example, in some embodiments in which the third transducer set that delivers the second high voltage pulse set per block 810A is the first transducer set that delivers the first high voltage pulse set per block 802A, the second high voltage pulse set may be temporally separated by a third high voltage pulse set in the uninterrupted high voltage pulse train deliverable by the first transducer set (which is the same as the third transducer set in this specific example) of the transducer-based device (e.g., 200, 300, or 400).

According to various embodiments, various time intervals may separate the delivery of the second high voltage pulse set (e.g., per block 810A) from the delivery of the first high voltage pulse set (e.g., per block 802A). These varying intervening time intervals may occur in various embodiments in which each of the first high voltage pulse set and the second high voltage pulse set form part of an uninterrupted high voltage pulse train. These varying intervening time intervals may also occur in various embodiments in which the first high voltage pulse set forms at least part of a first high voltage pulse train and the second high voltage pulse set forms at least part of a second high voltage pulse train other than the first high voltage pulse train. In some embodiments, the first transducer set delivers each of the first high voltage pulse train and the second high voltage pulse train. In some embodiments, the first transducer set delivers the first high voltage pulse train and the third transducer set delivers the second high voltage pulse train, the third transducer set being other than the first transducer set.

In some embodiments, successive pulses in the first high voltage pulse set (e.g., delivered per block 802A) are temporally spaced according to a first period of time, and successive pulses in the second high voltage pulse set (e.g., delivered per block 810A) are temporally spaced according to a second period of time. According to various embodiments, the second high voltage pulse set is temporally separated from the first high voltage pulse set by a time interval that is greater than each of the first period of time and the second period of time. In some embodiments, a relatively large duration of time (e.g., relatively larger than: (a) a duration of time from inception to conclusion of a delivery of the first high voltage pulse set (e.g., per block 802A), (b) a duration of time from inception to conclusion of a delivery of the second high voltage pulse set (e.g., per block 810A), or each of (a) and (b)) separates the conclusion of the delivery of the first high voltage pulse set from the initiation of the delivery of the second high voltage pulse set. In some embodiments, the second high voltage pulse set is temporally separated from the first high voltage pulse set by a third high voltage pulse set. In some embodiments, the third high voltage pulse set may be delivered by the first transducer set that delivers the first high voltage pulse set per block 802A. In some embodiments, the third high voltage pulse set may be delivered by the third transducer set that delivers the second high voltage pulse set per block 810A. In some embodiments, the third high voltage pulse set may be delivered by a transducer set of the transducer-based device (e.g., 200, 300, or 400) other than the first transducer set or the third transducer set. In some embodiments, the third high voltage pulse set may be delivered by a transducer set of a second transducer-based device (e.g., 200, 300, or 400) other than the transducer-based device that includes the first transducer set.

In some embodiments, the data processing device system (e.g., 110, 310) may be configured at least by the program at least to cause, via the input-output device system (e.g., 120, 320), monitoring of a third data set indicative of proximity between a location of at least a first transducer in the first transducer set at least at an inception or conclusion of, or during the delivery of the first high voltage pulse set and a location of at least a second transducer in the third transducer set at least at an inception or conclusion of, or during the delivery of the second high voltage pulse set. In some embodiments, the data processing device system (e.g., 110, 310) is configured at least by the program at least to cause determination of the second quality of the lesion producible on the tissue by the second high voltage pulse set at least based on an analysis of the third data set. In some embodiments, the third transducer set of the transducer-based device (e.g., 200, 300, or 400) that delivers the second high voltage pulse set is the first transducer set of the transducer-based device (e.g., 200, 300, or 400) that delivers the first high voltage pulse set. In some embodiments, the third transducer set of the transducer-based device (e.g., 200, 300, or 400) that delivers the second high voltage pulse set is other than the first transducer set of the transducer-based device (e.g., 200, 300, or 400) that delivers the first high voltage pulse set. According to various embodiments, the third data set may indicate a separation between the location of at least the first transducer in the first transducer set at least at the inception or conclusion of, or during the delivery of the first high voltage pulse set and the location of at least the second transducer in the third transducer set at least at the inception or conclusion of, or during the delivery of the second high voltage pulse set. Separation between the two locations may impact the resulting lesion quality since such separation may cause the first high voltage pulse set and the second high voltage pulse set to be delivered to different tissue regions or cause a diminished amount of overlap in the respective regions that each of the first high voltage pulse set and the second high voltage pulse set are delivered to. Separation between the two locations may be caused at least by different factors including patient movement (movement of the heart due to cardiac cycle or pulmonary cycle) or movement of at least part of the transducer-based device (e.g., 200, 300, 400).

For example, FIG. 9G-1 illustrates a simplified view of the graphical representation 900A in a state in which a lesion marker 912-1, like one of the lesion markers 912 in FIG. 9D or 9E, was formed by a prior ablation performed by a first transducer of a first transducer set of a transducer-based device (e.g., 200, 300, 400) that delivered the first high voltage pulse set (e.g., per block 802A). In FIG. 9G-1, transducer graphical elements 905-1 and 905-2 are examples of transducer graphical elements 905 and correspond to transducers of the transducer-based device. In the state of FIG. 9G-1, the transducer corresponding to transducer graphical element 905-2 is delivering the second high voltage pulse set and, therefore, may be considered a second transducer of the third transducer set referred to in block 810A, according to some embodiments. FIG. 9G-2 illustrates a simplified view of the graphical representation 900A in a state (i.e., after the state of FIG. 9G-1) where relative movement has occurred between (a) the location on the tissue surface of the bodily cavity represented by lesion marker 912-1, and (b) the locations of the transducers corresponding to transducer graphical elements 905-1, 905-2. Such relative movement may occur at least in part by, e.g., (1) movement of the transducer-based device “downward” (in the context of viewing FIGS. 9G-1 and 9G-2 with their proper orientations where the text in such figures is oriented horizontally) with respect to the state of FIG. 9G-1, which would cause the transducers associated with transducer graphical elements 905-1, 905-2 to move to the state of FIG. 9G-2 below the location of the lesion represented by lesion marker 912-1, or (2) movement of the bodily cavity “upward” (in the context of viewing FIGS. 9G-1 and 9G-2 with their proper orientations) with respect to the state of FIG. 9G-1, which would cause the lesion corresponding to lesion marker 912-1 to move to the state of FIG. 9G-2 above the locations of the transducers corresponding to transducer graphical elements 905-1, 905-2, or both (1) and (2).

Regardless of the cause of movement between the states of FIGS. 9G-1 and 9G-2, the lesion marker 912-1, in the state of FIG. 9G-2, is partially shaded with shaded region 913a that indicates the cumulative effect on the tissue due to the second high voltage pulse set delivery from the state of FIG. 9G-1. A second lesion marker 912-2 is added in the state of FIG. 9G-2, and it corresponds to the location of the transducer associated with transducer graphical element 905-2 when it delivered the second high voltage pulse set in the state of FIG. 9G-1. In this regard, just as the transducer graphical element 905-2 overlapped the lesion marker 912-1 in the state of FIG. 9G-1, the new lesion marker 912-2 overlaps the lesion marker 912-1 in the state of FIG. 9G-2. Due to this overlapping, each of the lesion markers 912-1 and 912-2 represent two respective lesion qualities: a first quality represented by the region with no color fill in the respective lesion marker (such as region 913b in the case of lesion marker 912-2 and region 913c in the case of lesion marker 912-1), and a second quality represented by the darker-shaded region in the respective lesion marker (such as region 913a, which is in each of lesion markers 912-1 and 912-2). Region 913c in lesion marker 912-1 may represent the same or a different quality of lesion as that represented by region 913b, according to various embodiments. The second quality of lesion (e.g., represented by shaded region 913a) in FIG. 9G-2 represents the cumulative effect on the tissue due to the second high voltage pulse set delivery as compared to the state of FIG. 9G-1. While FIG. 9G-2 shows separate lesion markers 912-1 and 912-2 including an overlapping region 913a, some other embodiments may include a single combined lesion marker with variations in visual characteristics to show lesion area and cumulative effects.

In some embodiments associated with FIGS. 9G-1 and 9G-2, the transducer corresponding to transducer graphical element 905-2 is the transducer that delivered the first high voltage pulse set per block 802A in the state of FIG. 9G-1. In such a situation, it can be seen that the data processing device system (110, 310) may be configured to determine the second quality of the lesion per block 812A based at least on an analysis of a data set (e.g., referred to as a third data set in some embodiments, which may be from a device location tracking system (e.g., FIG. 2 or 3)), which may indicate a change in location of at least part of the transducer-based device (e.g., a change in location of the transducer corresponding to transducer graphical element 905-2 in this example) from a time of the delivery of the first high voltage pulse set (e.g., per block 802A and represented by FIG. 9G-1 in this example) to a time of the delivery of the second high voltage pulse set (e.g., per block 810A and represented by FIG. 9G-2 in this example).

In some embodiments associated with FIGS. 9G-1 and 9G-2, the transducer corresponding to transducer graphical element 905-1 is the transducer that delivered the first high voltage pulse set per block 802A, such that lesion marker 912-1 represents the location of the transducer corresponding to graphical element 905-1 when such transducer delivered the first high voltage pulse set. Accordingly, the state of FIG. 9G-1, in this example, represents a time after delivery of the first high voltage pulse set, where the transducer associated with transducer graphical element 905-1 has since moved to the right and down (using the proper perspective of FIG. 9G-1) from the location it was in when it formed the lesion corresponding to lesion marker 912-1. Accordingly, such transducer corresponding to transducer graphical element 905-1 may be considered a first transducer in the first transducer set that delivered the first high voltage pulse set per block 802A, and, in this example, the transducer corresponding to transducer graphical element 905-2 is considered an example of a second transducer in the third transducer set that delivers the second high voltage pulse set per block 810A. In at least such a situation, it can be seen that the data processing device system (110, 310) may be configured to determine the second quality of the lesion per block 812A based at least on an analysis of a data set (e.g., referred to as a third data set in some embodiments, which (a) may be based at least in part on information from a device location tracking system (e.g., FIG. 2 or 3), (b) may be based at least in part on known geometry of the transducer-based device, or may be based at least in part on both (a) and (b)), which may be indicative of proximity between a location of at least the first transducer corresponding to transducer graphical element 905-1 at least before (e.g., in predictive contexts), during, or after (e.g., at least when the location of the first transducer is the same after) the delivery of the first high voltage pulse set (e.g., per block 802A and the state of at least FIG. 9G-1 in this example) and a location of at least the second transducer corresponding to transducer graphical element 905-2 at least before, during, or after the delivery of the second high voltage pulse set (e.g., per block 810A and the state of at least FIG. 9G-2 in this example). In this regard, the location of the first transducer corresponding to transducer graphical element 905-1 before, during, or, in this example, immediately after (e.g., prior to subsequent movement after) delivery of the first high voltage pulse set may be represented by the location of lesion marker 912-1 in the state of FIG. 9G-1, and the location of the transducer corresponding to transducer graphical element 905-2 before, during, or, in this example, immediately after (e.g., prior to subsequent movement after) may be represented by the location of transducer graphical element 905-2 in the state of FIG. 9G-1, according to some embodiments.

According to various embodiments, at least part of the transducer-based device (e.g., 200, 300, or 400) may move during (i) the delivery of the first high voltage pulse set (e.g., per block 802A), (ii) the delivery of the second high voltage pulse set (e.g., per block 810A), or each of (i) and (ii). In some embodiments, at least part of the transducer-based device (e.g., 200, 300, or 400) may move between (i) and (ii). In some embodiments, the at least part of the transducer-based device (e.g., 200, 300, or 400) includes (iii) the first transducer set, (iv) the second transducer set, or each of (iii) and (iv).

In some embodiments, the data processing device system (e.g., 110, 310) may be configured at least by the program at least to cause, via the input-output device system, monitoring of a third data set indicative of movement of at least part of the transducer-based device (e.g., 200, 300, 400), the third data set indicating a change in location of at least part of the transducer-based device from a time of the delivery of the first high voltage pulse set (e.g., per block 802A) to a time of the delivery of the second high voltage pulse set (e.g., per block 810A). In some embodiments, the data processing device system is configured at least by the program to determine the second quality of the lesion based at least on an analysis of the third data set (e.g., per block 812B). For example, in some embodiments, the input-output device system (e.g., 120, 320) may include a device location tracking system (e.g., 260A, 260B). In some embodiments, the data processing device system (e.g., 110, 310) may be configured at least by the program at least to determine location information of at least part of the transducer-based device (e.g., 200, 300, 400) based at least on a first location signal set provided by the device location tracking system (e.g., 260A, 260B), the location information indicating a change in location of the at least part of the transducer-based device during the delivery of the second high voltage pulse set as compared to a location of the at least part of the transducer-based device during the delivery of the first high voltage pulse set, e.g., as discussed above with respect to the example of FIGS. 9G-1 and 9G-2. In some embodiments, the at least part of the transducer-based device (e.g., 200, 300, 400) includes the third transducer set of the transducer-based device. In some embodiments, the third transducer set of the transducer-based device (e.g., 200, 300, 400) is the first transducer set of the transducer-based device. In some embodiments, the third transducer set of the transducer-based device (e.g., 200, 300, 400) is other than the first transducer set of the transducer-based device.

As described above in this disclosure with respect to some embodiments associated with FIG. 9B, a first graphical element set including transducer graphical elements 905x is displayed in FIG. 9B and corresponds to a delivery of a first high voltage pulse set delivered by a first transducer set corresponding to the transducer graphical elements 905x. In some embodiments associated with FIG. 9F, a second graphical element set is displayed upon the additional delivery of a second high voltage pulse set by a third transducer set corresponding to the transducer graphical elements 905x in the state of FIG. 9F, the third transducer set being the same as the first transducer set in this example. In some embodiments, the second graphical element set including transducer graphical elements 905x in the state of FIG. 9F has an appearance that indicates an enhanced lesion quality (referred to in the KEY of FIG. 9F as “1ST ENHANCED LESION QUALITY”) compared to the first graphical element set including transducer graphical elements 905x in the state of FIG. 9B. This enhanced lesion quality is due, at least in part in some embodiments, to the cumulative nature of pulsed field ablation. In some embodiments, associated with FIGS. 9B and 9F, the first graphical element set of transducer graphical elements 905x displayed in FIG. 9B may correspond to states in which the first transducer set has a same degree of proximity with respect to a tissue surface region during the delivery of the first and second high voltage pulse sets (e.g., per blocks 802A and 810A).

In other embodiments, some movement of at least some part of the transducer-based device (e.g., 200, 300, 400) may occur between the delivery of the first high voltage pulse set (e.g., per block 802A) and the delivery of the second high voltage pulse set (e.g., per block 810A). In some cases, the movement may essentially be such that each of the first high voltage pulse set and the second high voltage pulse set is applied to a same or substantially the same tissue region, but the first high voltage pulse set and the second high voltage pulse set are applied under different proximity conditions with respect to the tissue region (e.g., as described above in this disclosure). In other cases, the movement may essentially be such that each of the first high voltage pulse set and the second high voltage pulse set do not completely overlap the same tissue region, or respective lesion portions formed by each of the first high voltage pulse set and the second high voltage pulse set do not completely overlap. This may occur, in some embodiments, when movement of the at least the part of the transducer-based device includes a component of movement laterally away from the tissue region, or a movement of the at least the part of the transducer-based device includes a positioning away from the tissue region and then a repositioning back toward the tissue region to apply the second high voltage pulse set. Such repositioning may occur, for example, if a user (e.g., health care practitioner) applies the first voltage pulse set to the tissue region and then positions at least part of the transducer-based device (e.g., 200, 300, 400) away from the tissue region (for example, as may be the case when moving from the state of FIG. 9B to the state of FIG. 9D or from the state of FIG. 9C to the state of FIG. 9E) and then the user decides that the quality of the lesion (represented by graphical elements 912 in FIGS. 9D and 9E) formed at the tissue region should be or can be further enhanced with the application of an additional high voltage pulse set. In some embodiments, this may require a repositioning of at least part of the transducer-based device (e.g., 200, 300, 400) back to a location in the vicinity of the lesion for the application of the additional high voltage pulse set. Such repositioning will typically not reposition the at least part of the transducer-based device (e.g., 200, 300, 400) exactly at the location of the previously formed lesion, and, consequently, the final quality of the lesion may be impacted by such differences in positions when applying the second or additional high voltage pulse set (e.g., applied by the first transducer set that applied the first high voltage pulse set or applied by another transducer set of the transducer-based device). Accordingly, the revised lesion quality may, in some embodiments, may be dependent on the third data set described above in both contexts of movement of the transducer-based device and distances between different transducers applying the first and second high voltage pulse sets (e.g., as discussed above with respect to the example of FIGS. 9G-1 and 9G-2). In some embodiments, relationship (1), relationship (2) or each of relationships (1) and (2), discussed above, may be modified to account for less than complete overlap conditions existing between the application of the first high voltage pulse set and the additional high voltage pulse set (e.g., the second high voltage pulse set in some embodiments).

It is noted that regardless of the presence or absence of movement of (a) the transducer-based device, (a) the tissue or body, or both (a) and (b) between the application of the first high voltage pulse set and the second high voltage pulse set, the first graphical element set indicating the first quality of lesion and the second graphical element indicating the second quality of lesion may be displayed in various manners. For example, in some embodiments, the data processing device system (e.g., 110, 310) may be configured at least by the program at least to cause the display of the second graphical element set indicating the determined second quality of the lesion by replacing the first graphical element set indicating the determined first quality of the lesion with the second graphical element set indicating the determined second quality of the lesion. For example, in some embodiments, various ones of the transducer graphical elements 905 or graphical elements 912 indicating the first quality of lesion may be replaced with different transducer graphical elements 905 or graphical elements 912 indicating the second quality of lesion. In some embodiments, various ones of the transducer graphical elements 905 or graphical elements 912 may have different visual characteristics indicating different lesion qualities. For example, various ones of the transducer graphical elements 905 or graphical elements 912 having a visual characteristic set indicating the first quality of lesion may have such visual characteristic set replaced, changed, or updated with a different visual characteristic set to indicate the second quality of lesion. In some embodiments, the displayed second graphical element set is distinct from the displayed first graphical element set. For example, in some embodiments, the second graphical element set may, in some embodiments, consist of one or more graphical elements not included in the first graphical element set, the one or more graphical elements alone, or in combination with the first graphical element set, indicating the second quality of the lesion, according to some embodiments.

Referring to FIG. 8C, a block 802B is included, according to some embodiments, which represents a configuration of the data processing device system (e.g., data processing device system 110 or 310) (e.g., configured according to a program) to cause, via an input-output device system (e.g., input-output device system 120 or 320), activation or operation of at least a first transducer set of a transducer-based device (e.g., 200, 300, or 400) to deliver first ablation energy to cause ablation of tissue. In some embodiments, the first ablation energy is pulsed field ablation energy. In some embodiments, the first ablation energy is thermal ablation energy (e.g., RF ablation energy). In this regard, it is noted that while some embodiments associated with block 802A in FIG. 8B pertain to the delivery of pulsed field ablation, some embodiments associated with block 802B in FIG. 8C pertain more generally to tissue ablation, which may include pulsed field ablation, thermal ablation, and other types of ablation according to various embodiments. According to various embodiments, each transducer (e.g., transducer 206, 306, or 406) in the first transducer set is configured to deliver high voltage pulses configured to cause pulsed field ablation of tissue (e.g., monopolar or bipolar pulsed field ablation of tissue). According to various embodiments, each transducer (e.g., transducer 206, 306, or 406) in the first transducer set is configured to deliver thermal energy configured to cause thermal ablation of tissue (e.g., monopolar RF ablation, bipolar RF ablation, or blended monopolar-bipolar RF ablation).

In FIG. 8C, according to some embodiments, block 804B represents a configuration of the data processing device system (e.g., data processing device system 110 or 310) (e.g., configured according to a program) to cause, via the input-output device system (e.g., input-output device system 120 or 320), monitoring of a data set indicative of proximity between a second transducer set of the transducer-based device (e.g., 200, 300, or 400) and a tissue surface in a bodily cavity. In this regard, block 804B may the same or similar to block 804A in FIG. 8B, discussed above, including the corresponding descriptions and possible implementations and characteristics of the first and second transducer sets according to some embodiments.

Like various embodiments associated with block 804A of FIG. 8B, the monitored first data set associated with block 804B of FIG. 8C is indicative of proximity between the second transducer set of the transducer-based device (e.g., 200, 300, or 400) and tissue surface of a bodily cavity. In this regard, the monitored first data set associated with block 804B may be indicative of contact between the second transducer set of the transducer-based device (e.g., 200, 300, or 400) and a tissue surface of a bodily cavity according to various embodiments. In this regard, the monitored first data set associated with block 804B may be indicative of separation between the second transducer set of the transducer-based device (e.g., 200, 300, or 400) and a tissue surface of a bodily cavity, according to various embodiments. The first data set indicative of proximity between the second transducer set and the tissue surface may be derived in various manners, according to various embodiments. For example, proximity including contact between the second transducer set and the tissue surface may be determined by various contact detecting techniques including those described above in this disclosure. Proximity including separation between the second transducer set and the tissue surface may be determined by various separation or spacing detecting techniques including those described above in this disclosure. Proximity detectors may take various forms (for example, as described above in this disclosure).

According to some embodiments, the data processing device system (e.g., 110, 310) may be configured at least by the program at least to cause, via the input-output device system (e.g., 120, 320), the monitoring of the first data set (e.g., per block 804B) at least prior to the delivery of the first ablation energy (e.g., per block 802B). In some embodiments, the data processing device system (e.g., 110, 310) may be configured at least by the program at least to cause, via the input-output device system (e.g., 120, 320), the monitoring of the first data set at least during the delivery of the first ablation energy. In some embodiments, the data processing device system (e.g., 110, 310) is configured at least by the program at least to cause, via the input-output device system (e.g., 120, 320), the monitoring of the first data set at least after the delivery of the first high voltage pulse set. Monitoring the first data set indicative of proximity between the second transducer set and the tissue surface after delivery of the first ablation energy may be beneficial at least in a context where the second transducer set is the first transducer set that delivers the first ablation energy, and it may then be beneficial to monitor that transducer set's proximity to the tissue surface in preparation for a next delivery of ablation energy (e.g., which, in some embodiments, may be associated with block 810B discussed in more detail below), according to some embodiments.

In FIG. 8C, according to some embodiments, block 806B represents a configuration of the data processing device system (e.g., data processing device systems 110 or 310) (e.g., according to a program) to cause, based at least on an analysis of the first data set, determination, at least in response to a first state in which the analysis of the first data set is indicative of a first degree of proximity between the second transducer set and the tissue surface, of a first quality of a lesion producible in the tissue by that first ablation energy. Various determinations of qualities of lesion are exemplified above in this disclosure.

In FIG. 8C, according to some embodiments, block 808B represents a configuration of the data processing device system (e.g., data processing device systems 110 or 310) (e.g., according to a program) to cause, via the input-output device system (e.g., input-output device systems 120, 320), at least in response to the determination per block 806B of the first quality of lesion producible in the tissue by the first high voltage pulse set, display of a graphical element set with a first visual characteristic set indicating the determined first quality of lesion. The first graphical element set may take various forms, according to various embodiments. For example, graphical elements such as graphical elements corresponding to transducers (e.g., graphical elements 905), graphical elements representing tissue regions undergoing or having undergone ablation (e.g., lesion markers) (e.g., 912) or any other suitable graphical element set configured to indicate lesion quality may be employed, according to various embodiments.

According to various embodiments, in FIG. 9B, a first graphical element set made up of or including transducer graphical elements 905x may be displayed according to block 808B of FIG. 8C, where the first visual characteristic set of the graphical element set made up of or including graphical elements 905x have a visual characteristic set indicating the determined first quality of lesion (as indicated by the “1ST LESION QUALITY” entry in the KEY in FIG. 9B). According to various embodiments, the first quality of lesion associated with the first graphical element set made up of or including graphical elements 905x is based on an analysis of a first data set (e.g., per block 806B) that is indicative of a first degree of proximity between the second transducer set and the tissue surface that indicates a degree of separation (i.e., no contact) between the second transducer set and the tissue surface. Other graphical element sets may also be displayed according to various embodiments according to some embodiments associated with block 808B of FIG. 8C. For example, in FIG. 9B a graphical element set made up of or including transducer graphical elements 905a, 905b and 905c is displayed according to block 808B of FIG. 8C, a first visual characteristic set of the graphical element set made up of or including graphical elements 905a, 905b and 905c having a visual characteristic set indicating the determined first quality of lesion (as indicated by the “OTHER LESION QUALITY” entry in the KEY in FIG. 9B), according to some embodiments. According to various embodiments, the first quality of lesion associated with the first graphical element set made up of or including graphical elements 905a, 905b, and 905c is based on an analysis of the first data set (e.g., per block 806B) that is indicative of a first degree of proximity (i.e., contact or separation) between the second transducer set and the tissue surface that indicates a degree of contact between the second transducer set and the tissue surface. According to some embodiments, different first graphical element sets are displayed concurrently. For example, in FIG. 9B, the first graphical element set made up of or including graphical elements 905x and the first graphical element set made up of or including graphical elements 905a, 905b and 905c are displayed concurrently.

In FIG. 8C, according to some embodiments, block 810B represents a configuration of the data processing device system (e.g., data processing device systems 110) (e.g., at least by the program) to cause, via the input-output device system (e.g., 110, 310), second operation of at least the first transducer set of the transducer-based device (e.g., 200, 300, or 400) to deliver second ablation energy to cause ablation of the tissue. In this regard, block 810B pertains to some embodiments in which the same transducer set that delivers the first ablation energy per block 802B also delivers the second ablation energy per block 810B. According to various embodiments, the delivery of the second ablation energy (e.g., per block 810B) occurs after the delivery of the first ablation energy (e.g., per block 802B). It is noted that while some embodiments associated with block 810A in FIG. 8B pertain to the delivery of pulsed field ablation, some embodiments associated with block 810B in FIG. 8C pertain more generally to tissue ablation, which may include pulsed field ablation, thermal ablation, and other types of ablation, according to various embodiments.

In FIG. 8C, according to some embodiments, block 812B represents a configuration of the data processing device system (e.g., data processing device systems 110) (e.g., at least by the program) to cause determination of a second quality of the lesion producible in the tissue. In some embodiments, the second quality of the lesion producible in the tissue indicates a cumulative effect on the tissue as a result of delivery of the first ablation energy (e.g., per block 802B) and the second ablation energy (e.g., per block 810B). In this regard, the determined second quality of the lesion may be an indication of an actual cumulative effect, e.g., at least in a case in which the first and second ablation energies have been delivered or in which the first ablation energy has been delivered and the second ablation energy is being delivered, or the determined second quality of the lesion may be an indication of a predicted cumulative effect, e.g., at least in a case in which the first ablation energy has been delivered, but the second ablation energy has not yet been delivered or has not yet been completely delivered, such that the determined second lesion quality predicts the cumulative effect on the tissue if the second ablation energy is delivered or completely delivered, according to some embodiments.

In FIG. 8C, according to some embodiments, block 814B represents a configuration of the data processing device system (e.g., data processing device systems 110) (e.g., at least by the program) to cause, via the input-output device system (e.g., 110, 310) and at least in response to determination (e.g., per block 812B) of the second quality of lesion producible in the tissue, display of the graphical element set with a second visual characteristic set indicating the determined second quality of the lesion. According to various embodiments, the second quality of lesion (e.g., indicated by the second visual characteristic set as per block 814B in some embodiments) indicates a greater degree of tissue damage as compared to the first quality of the lesion (e.g., indicated by the first visual characteristic set as per block 808B in some embodiments). According to various embodiments, the second quality of lesion (e.g., indicated by the second visual characteristic set as per block 814B in some embodiments) indicates a greater degree of lesion size as compared to the first quality of the lesion (e.g., indicated by the first visual characteristic set as per block 808B in some embodiments). According to various embodiments, the second quality of lesion (e.g., indicated by the second visual characteristic set as per block 814B in some embodiments) indicates a greater degree of lesion depth as compared to the first quality of the lesion (e.g., indicated by the first visual characteristic set as per block 808B in some embodiments). According to various embodiments, the second quality of the lesion producible in the tissue indicates a cumulative effect on the tissue as a result of at least delivery of the first high voltage pulse set (e.g., per block 802B) and the second high voltage pulse set (e.g., per block 810B).

According to some embodiments, the delivery of the first ablation energy (e.g., per block 802B) and the delivery of the second ablation energy (e.g., per block 810B) form part of an uninterrupted delivery of ablation energy deliverable by the first transducer set of the transducer-based device (e.g., 200, 300, 400). In some embodiments, the delivery of the second ablation energy occurs immediately after the delivery of the first ablation energy during the uninterrupted delivery of ablation energy. In some embodiments, delivery of third ablation energy occurs between the delivery of the first ablation energy and the delivery of the second ablation energy, the delivery of the first ablation energy, the delivery of the second ablation energy, and the delivery of the third ablation energy forming part of an uninterrupted delivery of ablation energy deliverable by the first transducer set of the transducer-based device (e.g., 200, 300, 400).

In some embodiments, the uninterrupted delivery of ablation energy is an uninterrupted delivery of radiofrequency (“RF”) ablation energy. As indicated above in this disclosure, thermal ablation (e.g., RF ablation) energy is delivered in order to set up an elevated temperature distribution within the tissue and the lesion quality is governed primarily beginning at the point at which steady-state temperatures are achieved. Accordingly, the quality of the lesion that is formed improves as the energy is continuously delivered. So long as the thermal ablation is not stopped after initial thermal energy is applied, such that the tissue does not cool down after reaching steady-state temperatures, the quality of the lesion generally improves with the delivery of additional thermal energy primarily while the temperature profile is approaching steady-state (e.g., the initial delivery of thermal ablation energy and the additional delivery of thermal ablation energy forming part of an uninterrupted delivery of thermal ablation energy), and, therefore, the improved quality of the lesion indicates a cumulative effect on the tissue as a result of at least delivery of the first thermal ablation energy and the additional thermal ablation energy in an uninterrupted delivery. An uninterrupted delivery of thermal ablation energy may include a duty-cycled delivery of thermal ablation energy according to some embodiments.

In some embodiments, there is an interruption between the delivery of the first ablative energy per block 802B and the delivery of the second ablative energy per block 810B, but the interruption is shorter than the cooling period of the affected tissue, such that there is still a cumulative effect on lesion quality in the tissue due to delivery of the second ablative energy per block 810B.

In some embodiments, the aforementioned uninterrupted delivery of ablation energy is an uninterrupted delivery of pulsed field ablation energy. As indicated above in this disclosure, pulsed field ablation has a generally cumulative effect on a tissue region with each high voltage pulse that is delivered regardless, at least in some estimations, of how much time has elapsed between the delivery of successive pulses.

According to various embodiments, the graphical element set displayed with the second visual characteristic second according to block 814B includes a change to at least one visual characteristic of the first visual characteristic set (e.g., referred to in block 808B) to indicate a change in lesion quality from the first quality of the lesion to the second quality of the lesion, e.g., due to the delivery of the second ablation energy (e.g., delivered per block 810B). According to various embodiments, the graphical element set displayed with the second visual characteristic second according to block 814B includes a change to at least one visual characteristic of the first visual characteristic set to indicate the cumulative effect on the tissue indicating the cumulative effect on the tissue at least as a result of delivery of the first ablation energy and the second ablation energy. According to various embodiments, the graphical element set displayed with the second visual characteristic second according to block 814B includes an addition of at least one visual characteristic to the first visual characteristic set to indicate a change in lesion quality from the first quality of the lesion due to the delivery of the second ablation energy. According to various embodiments, the graphical element set displayed with the second visual characteristic set according to block 814B includes an addition of at least one visual characteristic, such as the addition of a “plus sign” to each respective transducer graphical element, or other symbol or visual characteristic, to the first visual characteristic set to indicate the cumulative effect on the tissue as a result of at least delivery of the first ablation energy and the second ablation energy.

According to various embodiments associated with FIG. 9F, the graphical element set with the second visual characteristic set indicating the determined second quality of the lesion may be displayed as transducer graphical elements 905a, 905b, 905c having the “2ND ENHANCED LESION QUALITY” pattern compared to the state of FIG. 9B, according to some embodiments of block 814B of FIG. 8C. According to various embodiments associated with FIG. 9F, the graphical element set with the second visual characteristic set may indicate the cumulative effect on the tissue as a result of at least delivery of the first ablation energy and the second ablation energy, according to some embodiments of block 814B of FIG. 8C. In this regard, according to some embodiments, a graphical element set made up of or including transducer graphical elements 905x is displayed in FIG. 9F according to block 814B of FIG. 8C, a second visual characteristic set of the second graphical element set made up of or including graphical elements 905x having a second visual characteristic set indicating the determined second quality of lesion (as indicated by the “1ST ENHANCED LESION QUALITY” entry in the KEY in FIG. 9F). According to various embodiments, the graphical element set made up of or including transducer graphical elements 905x that is displayed according to block 814A in FIG. 9F includes the same graphical elements 905x that are included in the graphical element set displayed according to block 808B in FIG. 9B, but the first visual characteristic set of the first graphical element set of graphical elements 905x in FIG. 9B has changed to produce the graphical element set of graphical elements 905x with the second visual characteristic set in FIG. 9F. According to various embodiments, the second visual characteristic set of the graphical element set of graphical elements 905x indicates a second quality of the lesion producible in the tissue as a cumulative effect on the tissue as a result of at least delivery of the first high voltage pulse set per block 802B of FIG. 8C and delivery of the second high voltage pulse set per block 810B of FIG. 8C. It is noted that other embodiments described in this disclosure with respect to FIGS. 9B and 9F (e.g., various methods associated with FIGS. 8A and 8B) have been described with respect to pulsed field ablation. Nonetheless, other methods associated with various embodiments of FIG. 8C may employ other ablation modalities such as thermal ablation.

As indicated above in this disclosure, pulsed field ablation (“PFA”) does not typically cause thermal coagulum and, as such, transducers that are not in contact with tissue, but rather are separated from the tissue, may be employed to deliver high voltage pulses configured to cause PFA of tissue. PFA causes tissue ablation via irreversible electroporation, which requires that the tissue be exposed to the generated electric fields. Although these electric fields may be affected by an impedance difference between tissue and blood, the quality of the lesions that are formed is generally more robust to loss of immediate transducer-to-tissue contact. In the limit, if tissue and blood are considered to have the same conductivity, theory may indicate that for every additional millimeter of transducer-to-tissue separation that is experienced, approximately a millimeter of lesion depth reduction occurs for reasonable amounts of transducer-to-tissue separation (e.g., 1-3 mm) In this regard, PFA energy may be delivered to cause a lesion in tissue with varying degrees of separation from (i.e., no contact with) the tissue. Of course, the range of separation is dependent on PFA energy levels delivered and desired lesion depth, as well as on transducer, blood, and tissue characteristics, so different embodiments may have different ranges of acceptable transducer-to-tissue separation when performing PFA. The present inventors have determined that under various transducer-to-tissue separation conditions, greater amounts of PFA energy (i.e., as compared to transducer-to-tissue contact conditions) may be delivered to, among other things, enhance lesion quality under the various transducer-to-tissue separation conditions. It is noted that, unlike thermal ablations procedures, the application of greater amounts of PFA energy under various transducer-to-tissue separation conditions (i.e., as compared to transducer-to-tissue contact conditions) does not lead to deleterious effects like the formation of thermal coagulum in cardiac ablation procedures, for example.

FIG. 8D represents a configuration of the data processing device system (e.g., data processing device system 110 or 310) that is configured (e.g., configured according to a program) to apply a first total amount of PFA energy at least in response to a first state in which at least part (e.g., a particular part being considered) of a transducer-based device (200, 300, 400) is in contact with a tissue surface in a bodily cavity, and to apply a second total amount of PFA energy greater than the first total amount of PFA energy at least in response to a second state in which the part (e.g., the particular part being considered) of the transducer-based device (200, 300, 400) is separated from the tissue surface in the bodily cavity. FIG. 9A shows, according to various embodiments, a graphical representation of a transducer-based device in which various transducers are in proximity with a tissue surface, with a first set of the transducers being in contact with the tissue surface and a second set of the transducers being separated from the tissue surface. In this regard, according to some embodiments, if the particular part being considered of the transducer-based device 200, 300, 400 illustrated in FIG. 9A is the part corresponding to the illustrated transducers shown as being in contact with the tissue surface in FIG. 9A, then the first set of transducers may correspond to the first state or another state in which the part of the transducer-based device 200, 300, 400 is in contact with the tissue surface. According to some embodiments, if the particular part being considered of the transducer-based device 200, 300, 400 illustrated in FIG. 9A is the part corresponding to the illustrated transducers shown as being separated from the tissue surface in FIG. 9A, then the second set of transducers may correspond to the second state or another state in which the part of the transducer-based device 200, 300, 400 is separated from the tissue surface. It is noted that, in some embodiments, both the first state and the second state may concurrently exist if, for example, the particular part being considered as the transducer-based device 200, 300, 400 includes one or more transducers that are in tissue contact and one or more transducers that are separated from the tissue surface (for example, as shown in FIG. 9A). In this regard, further discussions below with respect to block 825-3 in FIG. 8D discuss a case in which the first state occurs concurrently with a third state in which a second part of the transducer-based device is separated from a surface of the tissue, the second part of the transducer-based device different than the part of the transducer-based device that is in contact with a surface of the tissue, according to some embodiments.

FIG. 8D shows configurations of the data processing device system to behave differently in association with different states respectively referred to at least by broken-line blocks 825-1, 825-2, and 825-3 within block 825. In this regard, any one, two, or all of the states and corresponding actions set forth in blocks 825-1, 825-2, and 825-3 may actually occur or be executed by the data processing device system (e.g., as in a method) in some embodiments, and, in the case where two or more of the states and corresponding actions referred to by blocks 825-1, 825-2, and 825-3 actually occur or are executed by the data processing device system, they may occur in any order, as illustrated for some embodiments by the double-headed arrows shown in FIG. 8D.

FIG. 8D includes, according to some embodiments, block 820, which represents a configuration of the data processing device system (e.g., data processing device system 110 or 310) (e.g., configured according to a program) to receive, via an input-output device system (e.g., input-output device system 120 or 320), a data set indicative of proximity between at least part of a transducer-based device (200, 300, 400) and tissue in a bodily cavity. According to some embodiments, the part of the transducer-based device may include at least one transducer (e.g., transducer 206, 306, or 406). According to various embodiments, the part of the transducer-based device may include at least one transducer (e.g., transducer 206, 306, or 406) that will be subsequently activated (e.g., per block 825 discussed in more detail below) by the method of FIG. 8D to deliver PFA energy. According to various embodiments, the part of the transducer-based device may include at least one transducer (e.g., transducer 206, 306, or 406) that will be not subsequently activated by the method of FIG. 8D to deliver PFA energy. In some embodiments, the part of the transducer-based device (200, 300, 400) is a physical part of the transducer-based device. In some embodiments, the data set directly indicates the proximity between the part of transducer-based device (200, 300, 400) and the tissue surface in the bodily cavity. In some embodiments, the data set indicates indirectly the proximity between the part of the transducer-based device (200, 300, 400) and the tissue surface. For example, the data set may indicate a position/location (or a position/location relative to the tissue surface) of a physical or virtual portion of the transducer-based device, and based on a known or determined spatial relationship between the physical or virtual portion of the transducer-based device and the part of the transducer-base device, the proximity of the part of the transducer based device (200, 300, 400) to the tissue surface may be determined (for example, as described earlier in this disclosure). As indicated above in this disclosure, in some embodiments, proximity may include contact with the tissue surface, while in some embodiments, proximity may include separation from the tissue surface.

Determination of whether the part of the transducer-based device (e.g., 200, 300, or 400) is in contact with the tissue in the bodily cavity or is separated from the tissue may occur in various manners including the proximity detection techniques described above in this disclosure. For example, in some embodiments, the input-output device system 120 or 320 may be configured to receive the data set at least in part from a contact sensing system. Various contact sensing systems employed by various embodiments are described above in this disclosure. For example, as described above, in some embodiments, the contact sensing system may include a force sensing system configured to determine a degree of contact force between the part of the transducer-based device and the tissue surface in the bodily cavity. As per another example, as described above, the contact sensing system may include a flow sensing system configured to determine a degree of contact between the part of the transducer-based device and the tissue surface in the bodily cavity. In some embodiments, the input-output device system 120, 320 may be configured to interface with a proximity sensor configured to determine a distance from the proximity sensor to the tissue surface, and the data set may be determined based at least on an analysis of a signal set provided by the proximity sensor. For example, in some embodiments, the proximity sensor is an ultrasonic sensor. In some embodiments, the transducer-based device 200, 300, 400 may include the proximity sensor. In some embodiments, the input-output device system (120, 320) is configured to interface with a device location tracking system (e.g., 260A, 260B), and the data processing device system (110, 310) may be configured at least by the program at least to cause, via the input-output device system (120, 320), reception of a location signal set from the device location tracking system, the location tracking system indicating a location of at least a portion of the transducer-based device 200, 300, 400. According to various embodiments, the data set may be derived at least in part from the location signal set (e.g., as described above in this disclosure). For example, in some embodiments, the location tracking system directly determines the location (e.g., relative to the tissue surface) of the part of the transducer-based device, and in this regard the portion of the transducer-based device may be considered to be the part of the transducer-based device. In some embodiments, the located portion of the transducer-based device is other than the part of the transducer-based device, and a location (e.g., relative to the tissue surface) of the part of the transducer-based device may be determined based on a predetermined or determined spatial relationship between the part of the transducer-based device and the portion of the transducer-based device. In some embodiments, the device location tracking system is configured to generate the location signal set at least in response to one or more electric fields producible by one or more devices of the device location tracking system (for example, as described above in this disclosure with respect to device location tracking system 260A). In some embodiments, the device location tracking system is configured to generate the location signal set at least in response to one or more magnetic fields producible by one or more devices of the device location tracking system (for example, as described above in this disclosure with respect to device location tracking system 260B).

The use of a device location tracking system to determine proximity information may be accomplished in various manners. For example, according to some embodiments, the data processing device system 110, 310 may be configured at least by the program at least to cause display, via the input-output device system 120, 320, of an envelope representing the bodily cavity (e.g., envelope 902 derived by a device location tracking system as shown in FIGS. 9A to 9F, or the envelope 904 in the form of an image such as a CT scan as shown in FIG. 9H) and a representation of the transducer-based device (e.g., also shown in FIGS. 9A to 9F and FIG. 9H) located in proximity to the envelope. According to some embodiments, the data processing device system (110, 310) may be configured at least by the program at least to derive the data set at least in part from an analysis of information corresponding to a distance between at least part of the representation of the transducer-based device and a portion of the envelope adjacent the at least part of the representation of the transducer-based device. In some embodiments, the input-output device system 120, 320 may be configured to interface with a device location tracking system (e.g., 260A, 260B), and the data processing device system 110, 310 may be configured at least by the program to perform the analysis of the information corresponding to the distance between the at least part of the representation of the transducer-based device and the portion of the envelope adjacent the at least part of the representation of the transducer-based device based at least on a location signal set provided by the device location tracking system. In some embodiments, the input-output device system 120, 320 may be configured to interface with a device location tracking system (e.g., 260A, 260B), and the data processing device system 110, 310 may be configured at least by the program at least to determine a location of the at least part of the representation of the transducer-based device based at least on a first location signal set provided by the device location tracking system, and to determine a location of the portion of the envelope adjacent the at least part of the representation of the transducer-based device based at least on a second location signal set provided by the device location tracking system. For example, according to some embodiments, the envelope 902 shown in FIGS. 9A to 9F may be derived from various location signal sets provided a device location tracking system (for example, as described above in this disclosure) and the location of the portion of the envelope adjacent the at least part of the representation of the transducer-based device may be derived at least from a particular signal set (e.g., the second signal set) of the various location signal sets that corresponds to the location of the portion of the envelope adjacent the at least part of the representation of the transducer-based device.

In FIG. 8D, according to some embodiments, block 825 represents a configuration of the data processing device system (e.g., data processing device system 110 or 310) (e.g., according to a program) to cause via the input-output device system 120, 320 and the transducer-based device 200, 300, 400, initiation and then termination of delivery of at least a train of pulses (e.g., a high voltage pulse train in some embodiments) in accordance with at least a particular pulse train parameter set at least in response to a particular state in which at least part of the data set received as per block 820 indicates a particular amount or type of proximity between the part of the transducer-based device 200, 300, 400 and the tissue surface in the bodily cavity. In some embodiments, at least a particular transducer set of the transducer-based device 200, 300, 400 is activated per block 825 (or a portion thereof) to cause the initiation and then termination of the delivery of a train of pulses. In some embodiments, the particular transducer set of the transducer-based device 200, 300, 400 includes multiple transducers, and the train of pulses is delivered between the multiple transducers in accordance with a bipolar activation. In some embodiments, the train of pulses is delivered between each transducer in the particular transducer set of the transducer-based device 200, 300, 400 and one or more external transducers in accordance with a monopolar activation. The delivery of the train of pulses in a blended monopolar/bipolar activation may occur in some embodiments. In some embodiments, the part of the transducer-based device 200, 300, 400 (i.e., whose proximity with the tissue surface in the bodily cavity was indicated by the data set in block 820) may include the particular transducer set. In some embodiments, the part of the transducer-based device 200, 300, 400 may include one or more transducers other than any transducer included in the particular transducer set. In some embodiments, the part of the transducer-based device 200, 300, 400 may include one or more ablation transducers other than any transducer included in the particular transducer set.

According to some embodiments, the train of pulses is configured to be delivered in accordance with the particular pulse train parameter set to cause pulsed field tissue ablation. According to some embodiments, the train of pulses is configured to be delivered by a PFA transducer set provided by the transducer-based device 200, 300, 400. According to some embodiments, each of block 825-1, block 825-2, and block 825-3 represents a possible implementation of at least part of block 825 in a respective state, according to some embodiments.

According to some embodiments, block 825-1 represents a configuration of the data processing device system (e.g., data processing device systems 110 or 310) (e.g., according to a program) to cause, via the input-output device system 120, 320 and the transducer-based device 200, 300, 400, initiation and then termination of delivery of a first train of pulses configured in accordance with a first pulse train parameter set at least in response to a first state in which at least part of the data set (e.g., received via block 820) indicates that the part of the transducer-based device 200, 300, 400 is in contact with a tissue surface in the bodily cavity. FIG. 10A illustrates a simplified example of one such first train of pulses 1002a for illustration purposes, according to some embodiments. Although FIG. 10A, as well as FIG. 10B and FIG. 10C show uniphasic pulses for ease of illustration, biphasic pulses may be used in some embodiments. According to various embodiments, a first activation time period (e.g., first activation time period 1004a in FIG. 10A) exists from the initiation of the delivery of the first train of pulses to the termination of the delivery of the first train of pulses (e.g., by the respective transducer(s)). According to some embodiments, the first train of pulses is caused to be delivered during the first activation time period in accordance with the first pulse train parameter set to cause pulsed field tissue ablation. In some embodiments, the first pulse train parameter set is configured to cause the first train of pulses to deliver a first total energy over the first activation time period. In this regard, the first pulse train parameter set may be stored in the memory device system 130, 330 and may indicate one or more of various characteristics or configurations of the first train of pulses, such as pulse voltage (e.g., pulse voltage 1008a in FIG. 10A), number of pulses (e.g., number of pulses 1006a in FIG. 10A), frequency (e.g., as illustrated by the space between pulses in FIG. 10A), pulse width (e.g., pulse width 1010a in FIG. 10A), or another characteristic, or a combination thereof, that inform the data processing device system 110, 310 about how to cause the respective transducer(s) (e.g., transducer(s) 206, 306, or 406, in some embodiments, of the part of the transducer-based device), via the input-output device system 120, 320, to produce or deliver the first train of pulses configured in accordance with the first pulse train parameter set, according to some embodiments. The same applies correspondingly for the second pulse train parameter set and the third pulse train parameter set discussed in more detail below with respect to blocks 825-2 and 825-3, according to various embodiments.

According to some embodiments, block 825-2 represents a configuration of the data processing device system (e.g., data processing device systems 110 or 310) (e.g., according to a program) to cause, via the input-output device system 120, 320 and the transducer-based device 200, 300, 400, initiation and then termination of delivery of a second train of pulses configured in accordance with a second pulse train parameter set at least in response to a second state in which the at least part of the data set indicates that the part of the transducer-based device is separated from the tissue surface in the bodily cavity. FIG. 10B illustrates a simplified example of one such second train of pulses 1002b for illustration purposes, according to some embodiments. According to various embodiments, a second activation time period (e.g., second activation time period 1004b in FIG. 10B) exists from the initiation of the delivery of the second train of pulses to the termination of the delivery of the second train of pulses (e.g., by the respective transducer(s)).

According to some embodiments, the second train of pulses is caused to be delivered during the second activation time period in accordance with the second pulse train parameter set to cause pulsed field tissue ablation. In some embodiments, the second pulse train parameter set is configured to cause the second train of pulses to deliver a second total energy over the second activation time period. According to various embodiments, the second pulse train parameter set is different than the first pulse train parameter set. For example, the second train of pulses 1002b in FIG. 10B has a different total number of pulses 1006b than the total number of pulses 1006a in FIG. 10A, which may be caused by the second pulse train parameter set and the first pulse train parameter set defining such different pulse counts for the second state and first state, respectively, according to some embodiments. According to various embodiments, the second total energy is greater than the first total energy. For example, although each individual pulse has the same characteristics (e.g., including pulse voltages 1008a, 1008b and pulse widths 1010a, 1010b) in the examples of FIGS. 10A and 10B, the example of FIG. 10B includes a greater total number of pulses 1006b for the second train of pulses 1002b than the number of pulses 1006a provided by the first train of pulses 1002a in FIG. 10A, according to some embodiments. The greater number of pulses in the second train of pulses 1002b results in the greater second total energy delivered by the second train of pulses 1002b compared to the first total energy delivered by the first train of pulses 1002a. However, other embodiments can vary total energy delivered by a train of pulses by varying one or more other characteristics of the train of pulses in addition to or in lieu of the total number of pulses delivered, such as by varying pulse voltage, or pulse width, according to various embodiments.

In some embodiments, block 825 may be considered to represent a configuration of the data processing device system (e.g., data processing device system 110 or 310) (e.g., according to a program) to vary the pulse train characteristics of a particular deliverable pulse train at least in response to the received data set indicative of proximity between the part of the transducer-based device 200, 300, 400 and the tissue surface in the bodily cavity. For example, in some embodiments associated with block 825-1, the first train of pulses may be configured in accordance with the first pulse train parameter set at least in response to the first state in which at least part of the data set indicates that the part of the transducer-based-device 200, 300, 400 is in contact with the tissue surface in the bodily cavity. In a similar manner, in some embodiments associated with block 825-2, the second train of pulses may be configured in accordance with the second pulse train parameter set at least in response to the second state in which at least part of the data set indicates that the part of the transducer-based-device 200, 300, 400 is separated from the tissue surface in the bodily cavity.

In some embodiments, block 825 may be considered to represent a configuration of the data processing device system (e.g., data processing device system 110 or 310) (e.g., according to a program) to vary the total energy delivered by a pulse train during an activation time period at least in response to different types of proximity as indicated by the data set received per block 820. In some embodiments, greater total energy is delivered by a pulse train during an activation time period when the received data set indicates separation between the part of the transducer-based device 200, 300, 400 and a tissue surface in the bodily cavity than when the received data set indicates contact between the part of the transducer-based device 200, 300, 400 and the tissue surface in the bodily cavity. Such embodiments may be motivated for different reasons including delivering greater amounts of PFA total energy (e.g., to achieve better lesion quality) in response to the “separated” state while taking advantage of the low thermal output associated with PFA during the delivery of these greater amounts of PFA total energy, which has a reduced risk of the deleterious effects such as thermal coagulum that are associated with thermal ablation techniques. Other factors besides the type of proximity (separation vs. contact), such as the degree of contact or the degree of separation may also be considered, however, to adjust the total energy delivered, as discussed in more detail below, according to some embodiments.

Variances between the first total energy and the greater second total energy may be achieved, according to various embodiments, in accordance with variances between the respective first pulse train parameter set and the respective second pulse train parameter set. For example, in some embodiments, the first pulse train parameter set is configured to cause the first train of pulses (e.g., deliverable according to block 825-1; first train of pulses 1002a in FIG. 10A provides a simplified example) to deliver a first total number of pulses (e.g., first total number of pulses 1002a) throughout the first activation time period (e.g., first activation time period 1004a) in response to the first state, and the second pulse train parameter set is configured to cause the second train of pulses (e.g., deliverable according to block 825-2; second train of pulses 1002b in FIG. 10B provides a simplified example) to deliver a second total number of pulses (e.g., second total number of pulses 1002b) throughout the second activation time period (e.g., second activation time period 1004b) in response to the second state. According to various embodiments, the second total number of pulses is greater than the first total number of pulses, as discussed above with respect to FIGS. 10A and 10B. Generally, the greater number of pulses delivered by a particular pulse train, the more total energy that is delivered by the particular pulse train. This is especially true in the case of block 825 (described above) when various pulse train parameters other than the pulse count are the same in each of the first pulse train and the second pulse train. In this regard, in some embodiments, the first pulse train parameter set may be configured to cause each pulse in the first train of pulses to have a first pulse configuration in response to the first state, and the second pulse train parameter set may be configured to cause each pulse in the second train of pulses to have a second pulse configuration in response to the second state, the second pulse configuration being the same as the first pulse configuration, as is the case in the examples of FIGS. 10A and 10B. In some embodiments, the first pulse configuration and the second pulse configuration may define a same pulse voltage, as is the case in the examples of FIGS. 10A and 10B. In some embodiments, the first pulse configuration and the second pulse configuration may define a same pulse width, as is the case in the examples of FIGS. 10A and 10B (e.g., pulse widths 1010a, 1010b are the same). It is noted that the pulse width of various multi-phasic pulses (e.g., biphasic pulses, triphasic pulses) may, in some embodiments include intra-phase time periods as described above in this disclosure. In various embodiments, each pulse in the first pulse train is configured to deliver a first amount of pulse energy and each pulse in the second pulse train is configured to deliver a second amount of pulse energy nominally equal to the first pulse energy, as is the case in the examples of FIGS. 10A and 10B.

Changes to other pulse train parameter variables may alternatively or additionally be employed to achieve the variances between the first total energy and the greater second total energy. In some embodiments, the first pulse train parameter set may be configured to cause each of at least some of the pulses in the first train of pulses to have a first voltage in response to the first state, and the second pulse train parameter set is configured to cause each of at least some pulses in the second train of pulses to have a second voltage, the second voltage greater than the first voltage. For example, although the first and second trains of pulses 1002a, 1002b in FIGS. 10A, 10B respectively have the same pulse voltage for each pulse, other embodiments may have one or more of the pulses in the second train of pulses 1002b have an increased pulse voltage to increase the total energy of such second train of pulses 1002b in the ‘separated-from-tissue’ state as compared to the ‘tissue contact’ state. FIG. 10C, discussed in more detail below, shows a train of pulses 1002c having the same characteristics as train of pulses 1002a in FIG. 10A (e.g., including numbers of pulses 1006a, 1006c and pulse widths 1010a, 1010c), except for an increased pulse voltage 1008c compared to the pulse voltage 1008a of the first train of pulses 1002a. In some embodiments, the first pulse train parameter set may be configured to cause each of at least some pulses in the first train of pulses to have a first pulse width in response to the first state, and the second pulse train parameter set is configured to cause each of at least some pulses in the second train of pulses to have a second pulse width in response to the second state, the second pulse width greater than the first pulse width. Changes in pulse train parameters such as voltage and pulse width can be employed to increase the pulse energy deliverable by a pulse train during a particular activation time period, and hence be employed to increase the total energy delivered by a pulse train such as the second pulse train.

Other pulse train parameters variables may also be employed to achieve the variances between the first total energy and the greater second total energy according to various embodiment. For example, in some embodiments, the first pulse train parameter set may be configured to cause pulses in at least a portion of the first train of pulses to be delivered with a first pulse frequency in response to the first state, and the second pulse train parameter set may be configured to cause pulses in at least a portion of the second train of pulses to be delivered with a second pulse frequency in response to the second state, the second pulse frequency greater than the first pulse frequency. In this regard, the inter-pulse spacing between the pulses in the second train of pulses may be shorter than the inter-pulse spacing between pulses in the first train of pulses, and according to some embodiments, the second train of pulses may deliver more pulses, and an associated second total energy that is greater than the number of pulses and the associated total energy delivered by the first train of pulses.

According to various embodiments, the first activation time period (e.g., first activation time period 1004a in FIG. 10A) defines a duration of the first train of pulses deliverable in response to the first state (e.g., the first state referred to in block 825-1), the duration of the first train of pulses extending from the initiation of the first train of pulses to the termination of the first train of pulses. According to various embodiments, the second activation time period (e.g., second activation time period 1004b in FIG. 10B) defines a duration of the second train of pulses deliverable in response to the second state (e.g., the first state referred to in block 825-2). In some embodiments, the duration of the second train of pulses extends from the initiation of the second train of pulses to the termination of the second train of pulses. The initiation of the first train of pulses or the second train of pulses may occur in various ways. In some embodiments, the initiation of (a) the delivery of the first train of pulses, or (b) the delivery of the second train of pulses may be made at least in response to user instruction provided via the input-output device system 120, 320. In this regard, in some embodiments associated with block 825-1, a delivery of the first train of pulses configured according to the first pulse train parameter set at least in response to the data set (e.g., the data set received per block 820) indicating contact between the part of the transducer-based device and the tissue surface may be initiated at least in response to some particular user input requesting the initiation. In some embodiments associated with block 825-2, a delivery of the second train of pulses configured according to the second pulse train parameter set at least in response to the data set indicating separation between the part of the transducer-based device and the tissue surface may be initiated at least in response to some particular user input requesting the initiation. In this regard, a user may dictate when a particular pulse train configured for a particular set of proximity conditions should be delivered.

In some embodiments, the initiation of (a) the delivery of the first train of pulses, or (b) the delivery of the second train of pulses is made in response to a machine instruction provided according to the program. In this regard, a particular pulse train configured for a particular set of proximity conditions may be delivered automatically in some embodiments, for example, upon selecting or configuring a particular pulse train in accordance with the appropriate pulse train parameter set corresponding to the particular set of proximity conditions. It is noted that automatic initiation of a configured pulse train need not occur instantaneously, but may be delayed (for example, by a delay having a predetermined duration or by a delay that gates the delivery of the pulse train to a particular cardiac event or to a particular respiratory event, by way of non-limiting examples). In some embodiments, user-input may be required to affirm the machine-determined delivery of the configured pulse train.

According to various embodiments, the termination of (a) the delivery of the first train of pulses, or (b) the delivery of the second train of pulses, may be made in response to a machine instruction provided according to the program or the respective pulse train parameter set. It is noted, in some embodiments, that the termination of the delivery of the first train of pulses may correspond to a completion of a respective particular pre-determined or determined duration of time stored in the memory device system 130, 330 (e.g., as may be stored at least in part or in association with the first pulse train parameter set) that is commenced upon the initiation of the delivery of the first train of pulses. In a similar manner, in some embodiments, the termination of the delivery of the second train of pulses may correspond to a completion of a respective particular pre-determined or determined duration of time stored in the memory device system 130, 330 (e.g., as may be stored at least in part or in association with the second pulse train parameter set) that is commenced upon the initiation of the delivery of the second train of pulses. In some embodiments in which the first train of pulses is configured to deliver a particular number of pulses in response to the first state according to the first pulse train parameter set, a pulse counter may be employed to count the number of pulses and cause the termination of the first train of pulses when the particular number of pulses has been delivered. In a similar manner, in some embodiments in which the second train of pulses is configured to deliver a particular number of pulses in response to the second state according to the second pulse train parameter set, a pulse counter may be employed to count the number of pulses and cause the termination of the second train of pulses when the particular number of pulses has been delivered.

According to various embodiments, the first total energy deliverable by the first train of pulses (e.g., first train of pulses 1002a in the example of FIG. 10A) in response to the first state is dependent on the pulse energy delivered by each of the pulses in the first train of pulses and the duration of the first activation time period (e.g., first activation time period 1004a). In a similar manner, the second total energy deliverable by the second train of pulses (e.g., second train of pulses 1002b in the example of FIG. 10B) in response to the second state is dependent on the pulse energy delivered by each of the pulses in the second train of pulses and the duration of the second activation time period (e.g., second activation time period 1004b). In some embodiments, the first pulse train parameter set directly or indirectly defines a duration of the first activation time period. For example, in some embodiments, the first pulse train parameter set may directly define a duration of the first activation time period as a fixed time period from an initiation of the delivery of the first train of pulses to the termination of the of the first train of pulses. In some embodiments, the first pulse train parameter set may indirectly define a duration of the first activation time period, e.g., when a pre-determined or determined number of pulses have been delivered by the first pulse train. In a similar manner, the second pulse train parameter set may directly or indirectly define a duration of the second activation time period. In some embodiments, the first pulse train parameter set may define some aspect (e.g., a duration) of the first activation time period that is employed to configure a timing parameter set of the first train of pulses. In some embodiments, the second pulse train parameter set may define some aspect (e.g., a duration) of the second activation time period that is employed to configure a timing parameter set of the second train of pulses.

In some embodiments, a duration of the second activation time period is equal to a duration of the first activation time period. For example, in some embodiments, the first pulse train parameter set and the second pulse train parameter set may be configured to cause each of the first activation time period and the second activation time period to have the same duration when delivered in response to a respective one of the first state and the second state. Alternatively, each of the first activation time period and the second activation time period may be configured to have the same duration by program instructions that set the same duration of each of the first activation time period and the second activation time period independently of, or without reference to, the first pulse train parameter set and the second pulse train parameter set. When the durations of the first activation time period and the second activation time period are the same, pulse train parameters such as voltage, pulse width, the number of pulses (akin to pulse frequency in these embodiments) may be varied between the first train of pulses and the second train of pulses in accordance with the first pulse train parameter set and the second pulse train parameters set to cause the second total energy to be greater than the first total energy. When the durations of the first activation time period and the second activation time period are the same, the average power deliverable by the second train of pulses throughout the second activation time period is nominally greater than the average power deliverable by the first train of pulses throughout the first activation time period.

In some embodiments, a duration of the second activation time period is greater than a duration of the first activation time period. For example, in some embodiments, the first pulse train parameter set may be configured to cause, in response to the first state, a delivery of a particular first train of pulses that delivers a first number of pulses upon completion of the first activation time period, and the second pulse train characteristic set may be configured to cause, in response to the second state, a delivery of a particular second train of pulses that delivers a second number of pulses upon completion of the second activation time period, the second number of pulses being greater than the first number of pulses, as shown, e.g., in the example of FIGS. 10A and 10B. If the first train of pulses and the second train of pulses have a same pulse frequency, then the second activation time period (e.g., second activation time period 1004b) will be longer in duration than the first activation time period (e.g., first activation time period 1004a) to deliver the greater number of pulses that make up the second train of pulses. Other factors such as differences between the pulses of the first train of pulses and the pulses of the second train of pulses may also lead to a second activation time period that is longer in duration than the first activation time period. When the duration of the second activation time period is longer than the duration of the first activation time period, the average power deliverable by the second train of pulses throughout the second activation time period may or may not be greater than the average power deliverable by the first train of pulses throughout the first activation time period.

It is noted that, in some embodiments, the first train of pulses is configured (e.g., by the data processing device system 110, 310) in accordance with the first pulse train parameter set at least in response to the data set (e.g., the data set received per block 820 in FIG. 8D) indicating any degree of contact between the part of the transducer-based device 200, 300, 400 and the tissue surface in the bodily cavity. In some embodiments, the data set may include first data indicating a particular one of several possible degrees of contact between the part of the transducer-based device 200,300, 400 and the tissue surface in the bodily cavity, each of the several possible degrees of contact indicating some amount of contact between the part of the transducer-based device and the tissue surface in the bodily cavity. In some embodiments, the first pulse train parameter set may be configured to cause the first train of pulses to vary the first total energy delivered over the first activation time period in accordance with different degrees of contact between the part of the transducer-based device and the tissue surface in the bodily cavity indicated by the first data. In some embodiments, the first total energy, regardless of a manner in which it is varied according to the first pulse train parameter set in accordance with the different degrees of contact, is less than the second total energy. For example, in some embodiments, the first pulse train parameter set may itself include multiple subsets of one or more parameters, each subset associated with a respective range of degrees of contact. For instance, in some embodiments, the first pulse train parameter set may indicate a lower pulse voltage for the first train of pulses for a greater degree of tissue contact than for a lesser degree of contact, as less energy may be needed to cause ablation in the greater contact situation. Accordingly, in some embodiments, the first pulse train parameter set may be configured to cause the first train of pulses to vary in pulse voltage to vary the first total energy delivered over the first activation time period in accordance with the different degrees of contact between the part of the transducer-based device and the tissue surface in the bodily cavity indicated by the first data. In some embodiments, the first pulse train parameter set may be configured to cause the first train of pulses to vary in pulse width to vary the first total energy delivered over the first activation time period in accordance with the different degrees of contact between the part of the transducer-based device and the tissue surface in the bodily cavity indicated by the first data. In some embodiments, the first pulse train parameter set may be configured to cause the first train of pulses to vary in pulse frequency to vary the first total energy delivered over the first activation time period in accordance with the different degrees of contact between the part of the transducer-based device and the tissue surface in the bodily cavity indicated by the first data.

In some embodiments, the data set (e.g., the data set received per block 820 in FIG. 8D) may include second data indicating a particular one of several possible degrees of separation between the at least the part of the transducer-based device and the tissue surface in the bodily cavity, each of the several possible degrees of separation indicating some amount of separation between the part of the transducer-based device and the tissue surface in the bodily cavity. According to some embodiments, the second pulse train parameter set may be configured to cause the second train of pulses to vary the second total energy delivered over the second activation time period in accordance with different degrees of separation between the part of the transducer-based device and the tissue surface in the bodily cavity indicated by the second data. According to various embodiments, the second total energy, regardless of a manner in which it is varied according to the second pulse train parameter set in accordance with the different degrees of separation, is greater than the first total energy. For example, in some embodiments, the second pulse train parameter set may itself include multiple subsets of one or more parameters, each subset associated with a respective range of degrees of separation. For instance, in some embodiments, the second pulse train parameter set may indicate a higher pulse voltage for the second train of pulses for a greater degree of separation from tissue than for a lesser degree of separation from tissue, as more energy may be needed to cause ablation in the greater separation situation. Accordingly, in some embodiments, the second pulse train parameter set may be configured to cause the second train of pulses to vary in pulse voltage to vary the second total energy delivered over the second activation time period in accordance with the different degrees of separation between the part of the transducer-based device and the tissue surface in the bodily cavity indicated by the second data. In some embodiments, the second pulse train parameter set may be configured to cause the second train of pulses to vary in pulse width to vary the second total energy delivered over the second activation time period in accordance with the different degrees of separation between the part of the transducer-based device and the tissue surface in the bodily cavity indicated by the second data. In some embodiments, the second pulse train parameter set may be configured to cause the second train of pulses to vary in pulse frequency to vary the second total energy delivered over the second activation time period in accordance with the different degrees of separation between the part of the transducer-based device and the tissue surface in the bodily cavity indicated by the second data.

In some embodiments, it may be desired to activate (e.g., concurrently) each of multiple transducer sets to deliver respective pulse trains, the multiple transducer sets including (a) at least one transducer set that is in contact with a tissue surface, or is associated with the data set (e.g., the data set received per block 820 in FIG. 8D) indicating that a first part of the transducer-based device 200, 300, 400 is in contact with the tissue surface, and (b) at least one transducer set that is separated from the tissue surface, or is associated with the data set indicating that a second part of the transducer-based device 200, 300, 400 is separated from a tissue surface. For example, FIG. 9A indicates various transducers that are in contact with a tissue surface and various transducers that are separated from the tissue surface. In various embodiments, it may be desired that at least some of the “contacting” transducers be concurrently activated with at least some of the “separated” transducers to deliver respective pulse trains. In at least some of these embodiments, it may be desirable that each of the pulse trains delivered by the “separated” transducers deliver more energy than each of the pulse trains delivered by the “contacting” transducers. For example, in some embodiments, the part of the transducer-based device 200, 300, 400 may be a first part of the transducer-based device (for example, at least some of the “contacting” transducers shown in FIG. 9A), and the data processing device system 110, 310 may be configured at least by the program at least to cause, via the input-output device system 120, 320 and the transducer-based device, initiation and then termination of delivery of a third train of pulses configured in accordance with a third pulse train parameter set at least in response to a third state. In some embodiments, the third state is one in which a second part of the transducer-based device 200, 300, 400 other than the first part of the transducer-based device is separated from the tissue surface in the bodily cavity (for example, at least some of the “separated” transducers shown in FIG. 9A). According to various embodiments, the third state occurs concurrently with the first state. In this regard, FIG. 10A and FIG. 10C may represent an example of concurrently delivered pulse trains, where the first train of pulses 1002a in FIG. 10A is delivered for the “contacting” and the third train of pulses 1002c is delivered for the “separated” transducers according to a third pulse train parameter set. As shown in these figures, the third train of pulses 1002c is the same as the first train of pulses 1002a, but each pulse has a higher voltage 1008c in the third train of pulses 1002c compared to the pulse voltage 1008a of the first train of pulses 1002a, indicating the greater energy delivered in the “separated” third state compared to the “contacting” first state. Of course, one or more other pulse train parameters may be varied in addition to or in lieu of pulse voltage, according to various embodiments.

According to various embodiments, a third activation time period (e.g., third activation time period 1004c in FIG. 10C) exists from the initiation of the delivery of the third train of pulses to the termination of the delivery of the third train of pulses. According to various embodiments, the third train of pulses may be caused to be delivered during the third activation time period in accordance with the third pulse train parameter set to cause pulsed field tissue ablation. According to various embodiments, the third pulse train parameter set may be configured to cause the third train of pulses to deliver a third total energy over the third activation time period that is greater than the first total energy. According to various embodiments, the first activation time period and the third activation time period overlap.

While some of the embodiments disclosed above are described with examples of cardiac mapping, ablation, or both, the same or similar embodiments may be used for mapping, ablating, or both, other bodily organs, for example with respect to the intestines, the bladder, or any bodily organ to which the devices of the present invention may be introduced.

Subsets or combinations of various embodiments described above can provide further embodiments.

These and other changes can be made to the invention in light of the above-detailed description. In general, in the following claims, the terms used should not be construed to limit the invention to the specific embodiments disclosed in the specification and the claims, but should be construed to include other transducer-based device systems including all medical treatment device systems and all medical diagnostic device systems in accordance with the claims. Accordingly, the invention is not limited by the disclosure, but instead its scope is to be determined entirely by the following claims.

What is claimed is:

1. A transducer operation system comprising:
an input-output device system;
a memory device system storing a program; and
a data processing device system communicatively connected to the input-output device system and the memory device system, the data processing device system configured at least by the program at least to:
cause, via the input-output device system, an operation of at least a first transducer set of a transducer-based device to deliver a first high voltage pulse set to cause pulsed field ablation of tissue;
cause, via the input-output device system, monitoring of a first data set indicative of proximity between a second transducer set of the transducer-based device and a tissue surface in a bodily cavity;
cause, based at least on an analysis of the first data set, determination, at least in response to a first state in which the analysis of the first data set is indicative of a first degree of proximity between the second transducer set and the tissue surface, of a first quality of a lesion producible in the tissue by the first high voltage pulse set;
cause, via the input-output device system and at least in response to the determination of the first quality of the lesion producible in the tissue by the first high voltage pulse set, display of a first graphical element set indicating the determined first quality of the lesion;
cause, via the input-output device system, an operation of at least a third transducer set of the transducer-based device to deliver a second high voltage pulse set to cause pulsed field ablation of the tissue, the delivery of the second high voltage pulse set occurring after the delivery of the first high voltage pulse set;
cause determination of a second quality of the lesion producible in the tissue, the second quality of the lesion producible in the tissue indicating a cumulative effect on the tissue as a result of at least delivery of the first high voltage pulse set and the second high voltage pulse set; and
cause, via the input-output device system and at least in response to the determination of the second quality of the lesion, display of a second graphical element set indicating the determined second quality of the lesion.

2. The transducer operation system of claim 1, wherein the data processing device system is configured at least by the program at least to cause, via the input-output device system, monitoring of a second data set indicative of proximity between a fourth transducer set of the transducer-based device and the tissue surface of the bodily cavity, and wherein the data processing device system is configured at least by the program at least to cause the determination of the second quality of the lesion producible in the tissue based at least on an analysis of the second data set, the determination of the second quality of the lesion producible in the tissue made at least in response to a second state in which the analysis of the second data set is indicative of a second degree of proximity between the fourth transducer set and the tissue surface.

3. The transducer operation system of claim 2, wherein the data processing device system is configured at least by the program at least to cause the monitoring of the second data set to occur at least in part after the delivery of the first high voltage pulse set.

4. The transducer operation system of claim 2, wherein the fourth transducer set of the transducer-based device is the second transducer set of the transducer-based device.

5. The transducer operation system of claim 4, wherein the second degree of proximity between the fourth transducer set and the tissue surface is the same as the first degree of proximity between the second transducer set and the tissue surface.

6. The transducer operation system of claim 4, wherein the second degree of proximity between the fourth transducer set and the tissue surface is different than the first degree of proximity between the second transducer set and the tissue surface.

7. The transducer operation system of claim 4, wherein (a) the second degree of proximity between the fourth transducer set and the tissue surface indicates contact between at least one transducer in the fourth transducer set and the tissue surface, (b) the first degree of proximity between the second transducer set and the tissue surface indicates contact between at least one transducer in the second transducer set and the tissue surface, or each of (a) and (b).

8. The transducer operation system of claim 4, wherein (a) the second degree of proximity between the fourth transducer set and the tissue surface indicates separation between at least one transducer in the fourth transducer set and the tissue surface, (b) the first degree of proximity between the second transducer set and the tissue surface indicates separation between at least one transducer in the second transducer set and the tissue surface, or each of (a) and (b).

9. The transducer operation system of claim 4, wherein each of the second transducer set and the third transducer set is the first transducer set.

10. The transducer operation system of claim 4, wherein the fourth transducer set is the third transducer set.

11. The transducer operation system of claim 1, wherein the second quality of the lesion indicates an enhanced degree of quality as compared to the first quality of the lesion.

12. The transducer operation system of claim 1, wherein the second quality of the lesion indicates a greater degree of tissue damage as compared to the first quality of the lesion.

13. The transducer operation system of claim 1, wherein the second quality of the lesion indicates a greater degree of lesion size as compared to the first quality of the lesion.

14. The transducer operation system of claim 1, wherein the second quality of the lesion indicates a greater degree of lesion depth as compared to the first quality of the lesion.

15. The transducer operation system of claim 1, wherein the third transducer set of the transducer-based device is the first transducer set of the transducer-based device.

16. The transducer operation system of claim 1, wherein the third transducer set of the transducer-based device is other than the first transducer set of the transducer-based device.

17. The transducer operation system of claim 16, wherein the data processing device system is configured at least by the program at least to cause, via the input-output device system, monitoring of a third data set indicative of proximity between a location of at least a first transducer in the first transducer set at least at an inception or conclusion of, or during the delivery of the first high voltage pulse set and a location of at least a second transducer in the third transducer set at least at an inception or conclusion of, or during the delivery of the second high voltage pulse set, and wherein the data processing device system is configured at least by the program at least to cause determination of the second quality of the lesion producible in the tissue at least based on an analysis of the third data set.

18. The transducer operation system of claim 1, wherein the second graphical element set is the first graphical element set, but includes a change in at least one visual characteristic to indicate a change in lesion quality from the first quality of the lesion due to the delivery of the second high voltage pulse set.

19. The transducer operation system of claim 1, wherein the data processing device system is configured at least by the program at least to cause the display of the second graphical element set indicating the determined second quality of the lesion by replacing the first graphical element set indicating the determined first quality of the lesion with the second graphical element set indicating the determined second quality of the lesion.

20. The transducer operation system of claim 1, wherein the displayed second graphical element set is distinct from the displayed first graphical element set.

21. The transducer operation system of claim 1, wherein the data processing device system is configured at least by the program at least to cause, via the input-output device system, the monitoring of the first data set at least prior to the delivery of the first high voltage pulse set.

22. The transducer operation system of claim 1, wherein the data processing device system is configured at least by the program at least to cause, via the input-output device system, the monitoring of the first data set at least during the delivery of the first high voltage pulse set.

23. The transducer operation system of claim 1, wherein the data processing device system is configured at least by the program at least to cause, via the input-output device system, the monitoring of the first data set at least after the delivery of the first high voltage pulse set.

24. The transducer operation system of claim 2, wherein the data processing device system is configured at least by the program at least to cause, via the input-output device system, the monitoring of the second data set at least prior to the delivery of the second high voltage pulse set.

25. The transducer operation system of claim 2, wherein the data processing device system is configured at least by the program at least to cause, via the input-output device system, the monitoring of the second data set at least during the delivery of the second high voltage pulse set.

26. The transducer operation system of claim 2, wherein the data processing device system is configured at least by the program at least to cause, via the input-output device system, the monitoring of the second data set at least after the delivery of the second high voltage pulse set.

27. The transducer operation system of claim 1, wherein the cumulative effect on the tissue is a measured cumulative effect.

28. The transducer operation system of claim 1, wherein the cumulative effect on the tissue is a predicted cumulative effect.

29. The transducer operation system of claim 15, wherein the first high voltage pulse set and the second high voltage pulse set form part of an uninterrupted high voltage pulse train.

30. The transducer operation system of claim 29, wherein the second high voltage pulse set is temporally separated from the first high voltage pulse set by a third high voltage pulse set in the uninterrupted high voltage pulse train deliverable by the first transducer set of the transducer-based device.

31. The transducer operation system of claim 1, wherein the second high voltage pulse set is temporally separated from the first high voltage pulse set by a third high voltage pulse set.

32. The transducer operation system of claim 1, wherein successive pulses in the first high voltage pulse set are temporally spaced according to a first period of time, and successive pulses in the second high voltage pulse set are temporally spaced according to a second period of time, and wherein the second high voltage pulse set is temporally separated from the first high voltage pulse set by a time interval that is greater than each of the first period of time and the second period of time.

33. The transducer operation system of claim 1, wherein the input-output device system comprises a device location tracking system, and wherein the data processing device system is configured at least by the program at least to determine location information of at least part of the transducer-based device based at least on a first location signal set provided by the device location tracking system, the location information indicating a change in location of the at least part of the transducer-based device during the delivery of the second high voltage pulse set as compared to a location of the at least part of the transducer-based device during the delivery of the first high voltage pulse set.

34. The transducer operation system of claim 33, wherein the at least part of the transducer-based device comprises the third transducer set of the transducer-based device.

35. The transducer operation system of claim 34, wherein the third transducer set of the transducer-based device is the first transducer set of the transducer-based device.

36. The transducer operation system of claim 1, wherein the data processing device system is configured at least by the program at least to cause, via the input-output device system, monitoring of a third data set indicative of movement of at least part of the transducer-based device, the third data set indicating a change in location of at least part of the transducer-based device from a time of the delivery of the first high voltage pulse set to a time of the delivery of the second high voltage pulse set, and wherein the data processing device system is configured at least by the program to determine the second quality of the lesion based at least on an analysis of the third data set.

37. A method executed by a data processing device system according to a program stored by a communicatively connected memory device system, the data processing device system also communicatively connected to an input-output device system, and the method comprising:
operating, via the input-output device system, at least a first transducer set of a transducer-based device to deliver a first high voltage pulse set to cause pulsed field ablation of tissue;
monitoring, via the input-output device system, a first data set indicative of proximity between a second transducer set of the transducer-based device and a tissue surface in a bodily cavity;
determining, based at least on an analysis of the first data set and at least in response to a first state in which the analysis of the first data set is indicative of a first degree of proximity between the second transducer set and the tissue surface, a first quality of a lesion producible in the tissue by the first high voltage pulse set;
displaying, via the input-output device system and at least in response to the determination of the first quality of the lesion producible in the tissue by the first high voltage pulse set, a first graphical element set indicating the determined first quality of the lesion;
operating, via the input-output device system, at least a third transducer set of the transducer-based device to deliver a second high voltage pulse set to cause pulsed field ablation of the tissue, the delivery of the second high voltage pulse set occurring after the delivery of the first high voltage pulse set;
determining a second quality of the lesion producible in the tissue, the second quality of the lesion producible in the tissue indicating a cumulative effect on the tissue as a result of at least delivery of the first high voltage pulse set and the second high voltage pulse set; and
displaying, via the input-output device system and at least in response to the determination of the second quality of the lesion, a second graphical element set indicating the determined second quality of the lesion.

38. One or more non-transitory computer-readable storage mediums storing a program executable by a data processing device system communicatively connected to an input-output device system, the program comprising:
first operation instructions configured to cause, via the input-output device system, an operation of at least a first transducer set of a transducer-based device to deliver a first high voltage pulse set to cause pulsed field ablation of tissue;
monitoring instructions configured to cause, via the input-output device system, monitoring of a first data set indicative of proximity between a second transducer set of the transducer-based device and a tissue surface in a bodily cavity;
first determination instructions configured to cause, based at least on an analysis of the first data set, determination, at least in response to a first state in which the analysis of the first data set is indicative of a first degree of proximity between the second transducer set and the tissue surface, of a first quality of a lesion producible in the tissue by the first high voltage pulse set;
first display instructions configured to cause, via the input-output device system and at least in response to the determination of the first quality of the lesion producible in the tissue by the first high voltage pulse set, display of a first graphical element set indicating the determined first quality of the lesion;
second operation instructions configured to cause, via the input-output device system, an operation of at least a third transducer set of the transducer-based device to deliver a second high voltage pulse set to cause pulsed field ablation of the tissue, the delivery of the second high voltage pulse set occurring after the delivery of the first high voltage pulse set;
second determination instructions configured to cause determination of a second quality of the lesion producible in the tissue, the second quality of the lesion producible in the tissue indicating a cumulative effect on the tissue as a result of at least delivery of the first high voltage pulse set and the second high voltage pulse set; and
second display instructions configured to cause, via the input-output device system and at least in response to the determination of the second quality of the lesion, display of a second graphical element set indicating the determined second quality of the lesion.
